(19) United States
(12) Patent Application Publication
(10) Pub. No.: US 2020/0155609 A1
(43) Pub. Date: May 21, 2020

(54) COMPOSITIONS AND METHODS OF MAKING EXPANDED HEMATOPOIETIC STEM CELLS USING PTEN INHIBITORS

(71) Applicant: Transfusion Health LLC, South San Francisco, CA (US)

(72) Inventors: Timothy Webb, Ontario (CA); Zhan Wang, San Francisco, CA (US)

(21) Appl. No.: 16/615,389

(22) PCT Filed: May 18, 2018
(86) PCT No.: PCT/US2018/033389

§ 371 Date: Nov. 20, 2019

Related U.S. Application Data
(60) Provisional application No. 62/509,631, filed on May 22, 2017.
(60) Provisional application No. 62/578,308, filed on Oct. 27, 2017.
(60) Provisional application No. 62/583,321, filed on Nov. 8, 2017.

(51) Int. Cl. A61K 35/28; C12N 5/0789; C07C 233/65; C07C 225/32; C07C 233/43; C07C 233/33; C07C 50/34; C07C 233/25; C07C 233/29

(52) U.S. Cl. CPC A61K 35/28; C12N 5/0647; C07C 233/65; C07C 225/32; C07C 233/43; A61K 2035/124; C07C 50/34; C07C 233/25; C07C 233/29; C12N 2501/999; C07C 233/33

(57) ABSTRACT

This invention is directed to, inter alia, compounds, methods, systems, and compositions for the maintenance, enhancement, and expansion of hematopoietic stem cells derived from sources of non-mobilized peripheral blood. Also provided herein are compounds of Formula I

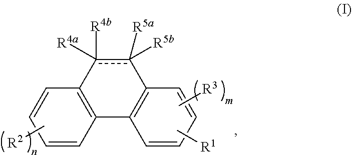

which are useful in maintaining, enhancing, and expanding of hematopoietic stem cells.

### CROSS-REFERENCES TO RELATED APPLICATIONS

This application claims priority of U.S. Provisional Patent Application No. 62/509,631, filed May 22, 2017; U.S. Provisional Patent Application No. 62/578,308, filed Oct. 27, 2017; and U.S. Provisional Patent Application No. 62/583,321, filed Nov. 8, 2017, the entire contents of each are incorporated by reference herein in their entireties.

### FIELD OF INVENTION

This invention is directed to, inter alia, methods and systems for maintaining and/or enhancing the expansion of hematopoietic stem cells and progenitors in culture, media for culturing hematopoietic stem cells and progenitors, and therapeutic compounds and compositions comprising the same for treatment of hematologic disorders.

### BACKGROUND OF THE INVENTION

The maintenance of the hematopoietic system relies on primitive pluripotent hermatopoietic stem cells (HSCs) that have the capacity to self-renew and repopulate all the blood cell lineages with relevant progenitor cells. Due to their capacity for self-renewal and their pluripotent, long term reconstituting potential, HSCs have long been considered ideal for transplantation to reconstitute the hematopoietic system after treatment for various hematologic disorders or as a target for the delivery of therapeutic genes. Additionally, human HSCs have potential applications for restoring the immune system in autoimmune diseases and in the induction of tolerance for allogenic solid organ transplantation.

The classical hematopoietic expansion cytokines thrombopoietin (TPO), stem cell factor (SCF), interleukin-3 (IL-3) and fms-related tyrosine kinase 3 ligand (Flt31) are insufficient for the true maintenance and expansion of HSCs. In these cultures, HSCs generally lose their potency within a week. Cord blood may be one of the best sources for HSCs available due to the relative potency of the cells and ease of access. Cord blood banks have extensive, preserved stocks of cells that can be rapidly employed for therapeutic use. However, without extensive expansion of a single cord unit, each cord is unlikely to be used for more than one therapeutic dose or application.

Considering the therapeutic benefits that maintenance and expansion, or enhancement of HSCs and/or early hematopoietic progenitor cells would enable, it is critical that new, aggressive, efficient, yet safe protocols and reagents be developed to meet this goal. The present disclosure addresses this need and provides related advantages as well.

Throughout this specification, various patents, patent applications and other types of publications (e.g., journal articles, electronic database entries, etc.) are referenced. The disclosure of all patents, patent applications, and other publications cited herein are hereby incorporated by reference in their entirety for all purposes.

### SUMMARY

Provided herein, inter alia, are compounds, methods and compositions for the rapid expansion, maintenance, and enhancement of hematopoietic stem cells and progenitors derived from one or more sources of CD34+ cells.

Accordingly, in some aspects, provided herein are compounds of Formula I

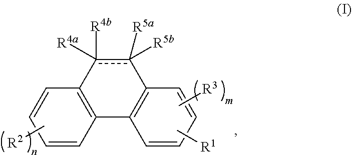

wherein R¹, R², R³, R^{4a}, R^{4b}, R^{5a}, R^{5b}, m, and n are as defined below.

Additionally, in some aspects, provided herein are methods for expanding hematopoietic stem cells and/or progenitors in culture, the method including contacting a source of CD34+ cells in culture with an effective amount of a phosphatase and tensin homolog (PTEN) inhibitor. In some embodiments, the PTEN inhibitor is SF1670 or a chemically altered version thereof. In some embodiments, the method for expanding hematopoietic stem cells and progenitors in culture restricts retinoic acid signaling. In some embodiments, retinoic acid signaling is limited by using media that controls or reduces the amount of retinoic acid. In some embodiments, the media includes a retinoic acid receptor (RAR) inhibitor or modulator. In some embodiments, the RAR inhibitor is ER50891.

In some aspects, source of CD34+ cells is bone marrow, cord blood, mobilized peripheral blood, or non-mobilized peripheral blood. In some aspects, the source of CD34+ cells is non-mobilized peripheral blood. In some aspects, the source of CD34+ cells includes: (a) CD34+ hematopoietic progenitors; (b) CD34+ early hematopoietic progenitors and/or stem cells; (c) CD133+ early hematopoietic progenitors and/or stem cells; and/or (d) CD90+ early hematopoietic progenitors and/or stem cells.

In some aspects, the method stabilizes the hematopoietic stem cell phenotype. In some aspects, the hematopoietic stem cell phenotype includes: CD45+, CD34+, CD133+, CD90+, CD45RA−, CD38 low/−, and negative for major hematopoietic lineage markers including CD2, CD3, CD4, CD5, CD8, CD14, CD16, CD19, CD20, CD56. In some aspects, CD133+ and/or CD90+ positive cells are increased compared to cells in culture that are not contacted with a PTEN inhibitor. In some aspects, the cells exhibit at least about two times the number of CD133+ and/or CD90+ positive cells compared to cells in culture that are not contacted with a PTEN inhibitor. In some aspects, CD90+ cells are increased compared to cells in culture that are not contacted with a PTEN inhibitor. In some aspects, CD38 low/− cells are increased compared to cells in culture that are not contacted with a PTEN inhibitor. In some aspects, CD90+ and CD38 low/− cells are increased compared to cells in culture that are not contacted with a PTEN inhibitor. In some aspects, the source of the CD34+ cells is a human being. In some embodiments, the PTEN inhibitor is SF1670 or a chemically altered version thereof.

In some aspects, provided herein are methods for producing a cell culture medium for culturing hematopoietic stem cells (HSC) and/or progenitor cells. The method involves combining a base or a feed medium; and a PTEN inhibitor. In some embodiments, the PTEN inhibitor is SF1670 or a chemically altered version thereof.

In some aspects, provided herein are systems for maintaining and/or enhancing the expansion of hematopoietic stem cells in culture. This system includes a source of CD34+ cells in culture (such as a CD34+ cells from one or more of bone marrow, cord blood, mobilized peripheral blood, and non-mobilized peripheral blood) and any of the cell culture media compositions described herein.

In some aspects, provided herein are methods for treating an individual in need of hematopoietic reconstitution. The method involves administering to the individual a therapeutic agent containing any of the cultured HSCs derived according to the methods of the present invention.

### BRIEF DESCRIPTION OF THE DRAWINGS

FIG. 1A, FIG. 1B, and FIG. 1C show that SF1670 or SF1670 and ER50891 improves the maintenance and enhancement of the hematopoietic stem cell phenotype. For each referenced Figure, “Base conditions” (1) are shown with diamond data points connected with a solid line; “+SF Conditions” (2) are shown with square data points connected with a dashed line (small dashes); “+SF/+ER Conditions” (3) are shown with triangle data points connected with a dashed line (long dashes). FIGS. 1A, 1B, and 1C show that addition of SF1670 or SF1670 and ER50891 increases the number of CD34+ cells (1A), CD133+ cells (1B), and CD90+ cells (1C) after 7 days in culture at the indicated conditions.

FIG. 2A, FIG. 2B, and FIG. 2C show that SF1670 and ER50891 together in culture improves the maintenance and enhancement of the hematopoietic stem cell phenotype. For each referenced Figure, “+SF/+ER Conditions” are shown with triangle data points connected with a dashed line (long dashes). FIGS. 2A, 2B, and 2C show that addition of SF1670 and ER50891 together in culture increases the number of CD34+ cells (2A), CD133+ cells (2B), and CD90+ cells (2C) after 14 days in culture at the indicated conditions.

FIG. 3A, FIG. 3B, FIG. 3C, FIG. 3D, and FIG. 3E report flow cytometric cell counts in cord blood samples cultured in “Base conditions” (dark grey column, on left) and “+SF Conditions” (light grey column, on right). FIG. 3A reports the total number of live cells in culture, and FIGS. 3B, 3C, 3D, and 3E show that +SF conditions increase the total number of CD34+ cells (3B), CD133+ cells (3C), CD90+ cells (3D), and CD38− cells (3E).

FIG. 4A, FIG. 4B, FIG. 4C, FIG. 4D, and FIG. 4E report the fold change in cell counts from day 2 to the indicated day based on the cord blood data reported in FIG. 3. “Base conditions” is the dark grey column, on the left of each time point and “+SF Conditions” is light grey column, on the right. FIG. 4A reports the fold change of live cells in culture, and FIGS. 4B, 4C, 4D, and 4E show the fold change in the total number of CD34+ cells (4B), CD133+ cells (4C), CD90+ cells (4D), and CD38− cells (4E).

FIG. 5A, FIG. 5B, FIG. 5C, FIG. 5D, and FIG. 5E report flow cytometric cell counts of peripheral blood samples after day 4 (light grey column, on left) and day 7 (black column, on right) when cultured under varying SF1670 concentrations from “Donor A” (left panel) and “Donor B” (right panel). FIGS. 5A, 5B, 5C, 5D, and 5E show the effect of increasing SF1670 concentration on the expansion of total cells (5A), CD34+ cells (5B), CD133+ cells (5C), CD90+ cells (5D), and CD38− cells (5E).

FIG. 6 illustrates the expansive effect measured for Compound 1.001 (columns) and controls: basic conditions (thin dashed lines) and +SF conditions (thick dashed lines). The data is reported as the fold change from days 2 to 7 for all live cells (top left panel), CD34+ cells (top right panel), CD133+ cells (bottom left panel), and CD90+ cells (bottom right panel). Each column reports the fold change in cells at the noted concentration of Compound 1.001.

FIG. 7 illustrates the expansive effect measured for Compound 1.002 (columns) and controls: basic conditions (thin dashed lines) and +SF conditions (thick dashed lines). The data is reported as the fold change from days 2 to 7 for all live cells (top left panel), CD34+ cells (top right panel), CD133+ cells (bottom left panel), and CD90+ cells (bottom right panel). Each column reports the fold change in cells at the noted concentration of Compound 1.002.

FIG. 8 illustrates the expansive effect measured for Compound 1.003 (columns) and controls: basic conditions (thin dashed lines) and +SF conditions (thick dashed lines). The data is reported as the fold change from days 2 to 7 for all live cells (top left panel), CD34+ cells (top right panel), CD133+ cells (bottom left panel), and CD90+ cells (bottom right panel). Each column reports the fold change in cells at the noted concentration of Compound 1.003.

FIG. 9 illustrates the expansive effect measured for Compound 1.004 (columns) and controls: basic conditions (thin dashed lines) and +SF conditions (thick dashed lines). The data is reported as the fold change from days 2 to 7 for all live cells (top left panel), CD34+ cells (top right panel), CD133+ cells (bottom left panel), and CD90+ cells (bottom right panel). Each column reports the fold change in cells at the noted concentration of Compound 1.004.

FIG. 10 illustrates the expansive effect measured for Compound 1.005 (columns) and controls: basic conditions (thin dashed lines) and +SF conditions (thick dashed lines). The data is reported as the fold change from days 2 to 7 for all live cells (top left panel), CD34+ cells (top right panel), CD133+ cells (bottom left panel), and CD90+ cells (bottom right panel). Each column reports the fold change in cells at the noted concentration of Compound 1.005.

FIG. 11 illustrates the expansive effect measured for Compound 1.006 (columns) and controls: basic conditions (thin dashed lines) and +SF conditions (thick dashed lines). The data is reported as the fold change from days 2 to 7 for all live cells (top left panel), CD34+ cells (top right panel), CD133+ cells (bottom left panel), and CD90+ cells (bottom right panel). Each column reports the fold change in cells at the noted concentration of Compound 1.006.

FIG. 12 illustrates the expansive effect measured for Compound 1.007 (columns) and controls: basic conditions (thin dashed lines) and +SF conditions (thick dashed lines). The data is reported as the fold change from days 2 to 7 for all live cells (top left panel), CD34+ cells (top right panel), CD133+ cells (bottom left panel), and CD90+ cells (bottom right panel). Each column reports the fold change in cells at the noted concentration of Compound 1.007.

FIG. 13 illustrates the expansive effect measured for Compound 1.008 (columns) and controls: basic conditions (thin dashed lines) and +SF conditions (thick dashed lines). The data is reported as the fold change from days 2 to 7 for all live cells (top left panel), CD34+ cells (top right panel), CD133+ cells (bottom left panel), and CD90+ cells (bottom right panel). Each column reports the fold change in cells at the noted concentration of Compound 1.008.

FIG. 14 illustrates the expansive effect measured for Compound 1.009 (columns) and controls: basic conditions (thin dashed lines) and +SF conditions (thick dashed lines). The data is reported as the fold change from days 2 to 7 for all live cells (top left panel), CD34+ cells (top right panel), CD133+ cells (bottom left panel), and CD90+ cells (bottom right panel). Each column reports the fold change in cells at the noted concentration of Compound 1.009.

FIG. 15 illustrates the expansive effect measured for Compound 1.010 (columns) and controls: basic conditions (thin dashed lines) and +SF conditions (thick dashed lines). The data is reported as the fold change from days 2 to 7 for all live cells (top left panel), CD34+ cells (top right panel), CD133+ cells (bottom left panel), and CD90+ cells (bottom right panel). Each column reports the fold change in cells at the noted concentration of Compound 1.010.

FIG. 16 illustrates the expansive effect measured for Compound 1.011 (columns) and controls: basic conditions (thin dashed lines) and +SF conditions (thick dashed lines). The data is reported as the fold change from days 2 to 7 for all live cells (top left panel), CD34+ cells (top right panel), CD133+ cells (bottom left panel), and CD90+ cells (bottom right panel). Each column reports the fold change in cells at the noted concentration of Compound 1.011.

FIG. 17 illustrates the expansive effect measured for Compound 1.012 (columns) and controls: basic conditions (thin dashed lines) and +SF conditions (thick dashed lines). The data is reported as the fold change from days 2 to 7 for all live cells (top left panel), CD34+ cells (top right panel), CD133+ cells (bottom left panel), and CD90+ cells (bottom right panel). Each column reports the fold change in cells at the noted concentration of Compound 1.012.

FIG. 18 illustrates the expansive effect measured for Compound 1.013 (columns) and controls: basic conditions (thin dashed lines) and +SF conditions (thick dashed lines). The data is reported as the number of cells per well after 7 days in culture for all live cells (top left panel), CD34+ cells (top right panel), and CD133+ cells (bottom left panel). Each column reports the total number of cells at the noted concentration of Compound 1.013.

FIG. 19 illustrates the expansive effect measured for Compound 1.014 (columns) and controls: basic conditions (thin dashed lines) and +SF conditions (thick dashed lines). The data is reported as the number of cells per well after 7 days in culture for all live cells (top left panel), CD34+ cells (top right panel), CD133+ cells (bottom left panel), and CD90+ cells (bottom right panel). Each column reports the total number of cells at the noted concentration of Compound 1.014.

FIG. 20 illustrates the expansive effect measured for Compound 1.015 (columns) and controls: basic conditions (thin dashed lines) and +SF conditions (thick dashed lines). The data is reported as the number of cells per well after 7 days in culture for all live cells (top left panel), CD34+ cells (top right panel), CD133+ cells (bottom left panel), and CD90+ cells (bottom right panel). Each column reports the total number of cells at the noted concentration of Compound 1.015.

FIG. 21 illustrates the expansive effect measured for Compound 1.016 (columns) and controls: basic conditions (thin dashed lines) and +SF conditions (thick dashed lines). The data is reported as the number of cells per well after 5 days in culture for all live cells (top left panel), CD34+ cells (top right panel), CD133+ cells (bottom left panel), and CD90+ cells (bottom right panel). Each column reports the total number of cells at the noted concentration of Compound 1.016.

### DETAILED DESCRIPTION OF THE INVENTION

The invention described herein provides, inter alia, methods and compositions for the maintenance, enhancement, and expansion of hematopoietic stem cells (HSCs) derived from one or more sources of CD34+ cells (such as, non-mobilized peripheral blood). Peripheral blood is known to reliably carry a small number of CD34+ hematopoietic progenitors and an even smaller number of CD34+ and CD133+ early hematopoietic progenitors and stem cells. Being the source with the least potent, least enriched, most dilute and impractically small numbers of apparent stem cells by nature, stem cell scientists have generally concluded that this source is unlikely to be therapeutically relevant compared to other potential sources of HSCs, such as bone marrow cells, mobilized peripheral blood, cord blood, and even embryonic or induced pluripotent stem cell (also known as iPS)-sourced CD34+ cells. Despite failed efforts to expand blood stem cells using more potent sources of cells, such as bone marrow and cord blood, there is some evidence that mitogenic, survival promoting, and quiescence inducing factors can impact the phenotype of these cells in positive ways and even help maintain them for some time in vitro.

The inventors of the present invention have observed that multipotent blood stem cells and progenitors can be successfully maintained, expanded, and enhanced by culturing these cells in a medium containing a PTEN inhibitor. In some embodiments the PTEN inhibitor is SF1670 or a chemically altered version thereof. In particular, the methods and compositions of the present invention are not only able to successfully derive HSCs from conventional sources, such as bone marrow, cord blood, and mobilized peripheral blood, but also from non-conventional sources such as non-mobilized peripheral blood. As such, the methods and compositions described herein provide for the generation of a therapeutically relevant stem cell transplant product derived from an easy to access and permanently available tissue source, without the need to expose the donor to significant risk or pain and which is more readily available than cord blood.

### I. General Techniques

The practice of the present invention will employ, unless otherwise indicated, conventional techniques of molecular biology, microbiology, cell biology, biochemistry, nucleic acid chemistry, and immunology, which are well known to those skilled in the art. Such techniques are explained fully in the literature, such as, Molecular Cloning: A Laboratory Manual, fourth edition (Sambrook et al., 2012) and Molecular Cloning: A Laboratory Manual, third edition (Sambrook and Russel, 2001), (jointly referred to herein as “Sambrook”); Current Protocols in Molecular Biology (F. M. Ausubel et al., eds., 1987, including supplements through 2014); PCR: The Polymerase Chain Reaction, (Mullis et al., eds., 1994); Antibodies: A Laboratory Manual, Second edition, Cold Spring Harbor Laboratory Press, Cold Spring Harbor, N.Y. (Greenfield, ed., 2014), Beaucage et al. eds., Current Protocols in Nucleic Acid Chemistry, John Wiley & Sons, Inc., New York, 2000, (including supplements through 2014), Gene Transfer and Expression in Mammalian Cells (Makrides, ed., Elsevier Sciences B.V., Amsterdam, 2003), and Current Protocols in imnunology (Horgan K and S. Shaw (1994) (including supplements through 2014).

### II. Definitions

Hematopoietic cells encompass not only HSCs, but also erythrocytes, neutrophils, monocytes, platelets, megakaryocytes, mast cells, eosinophils and basophils, B and T lymphocytes and NK cells as well as the respective lineage progenitor cells.

As used herein, “maintaining the expansion” of HSCs refers to the culturing of these cells such that they continue to divide rather than adopting a quiescent state and/or losing their multipotent characteristics. Multipotency of cells can be assessed using methods known in the art using known multipotentcy markers. Exemplary multipotency markers includes CD133+, CD90+, CD38 low/−, CD45RA negativity but overall CD45 positivity, and CD34. In some examples, CD34 low/− cells may be hematopoietic stem cells. In examples, where CD34 low/− cells are hematopoietic stem cells, these cells express CD133.

As used herein the term “cytokine” refers to any one of the numerous factors that exert a variety of effects on cells, for example, inducing growth or proliferation. The cytokines may be human in origin, or may be derived from other species when active on the cells of interest. Included within the scope of the definition are molecules having similar biological activity to wild type or purified cytokines, for example produced by recombinant means; and molecules which bind to a cytokine factor receptor and which elicit a similar cellular response as the native cytokine factor.

The term “culturing” refers to the propagation of cells on or in media (such as any of the media disclosed herein) of various kinds.

As used herein, the term “mobilized blood” refers to cells which have been exposed to an agent that promotes movement of the cells from the bone marrow into the peripheral blood and/or other reservoirs of the body (e.g., synovial fluid) or tissue.

As used herein, the phrase “non-mobilized peripheral blood” refers to a blood sample obtained from an individual who has not been exposed to an agent that promotes movement of the cells from the bone marrow into the peripheral blood and/or other reservoirs of the body. In some cases, “non-mobilized peripheral blood” refers to the blood from an individual who has not been exposed to an agent that promotes movement of the cells from the bone marrow into the peripheral blood and/or other reservoirs of the body for at least 1, 3, 5, 7, or 10 or more days. In some cases, “non-mobilized peripheral blood” refers to the blood of individuals who have not been exposed to an agent that promotes movement of the cells from the bone marrow into the peripheral blood and/or other reservoirs of the body for at least 5, 7, 10, 14, 21 or more days. In some cases, “non-mobilized peripheral blood” refers to the blood of individuals who have not been exposed to an agent that promotes movement of the cells from the bone marrow into the peripheral blood and/or other reservoirs of the body for at least 14, 21, 28, 35, 42, 49 or more days.

“Tetraspanins,” (also called “tetraspans” or “the transmembrane 4 superfamily” (TM4SF)) as used herein, refer to a family of membrane proteins found in all multicellular eukaryotes that have four transmembrane domains, intracellular N- and C-termini and two extracellular domains: one called the small extracellular domain or loop (SED/SEL or EC1) and the other, longer (typically 100 amino acid residue), domain called the large extracellular domain/loop (LED/LEL or EC2). There are 34 tetraspanins in mammals, 33 of which have also been identified in humans. Tetraspanins display numerous properties that indicate their physiological importance in cell adhesion, motility, activation and proliferation, as well as their contribution to pathological conditions such as metastasis or viral infection.

An “individual” can be a vertebrate, a mammal, or a human. Mammals include, but are not limited to, farm animals, sport animals, pets, primates, mice and rats. In one aspect, an individual is a human.

“Treatment,” “treat,” or “treating,” as used herein covers any treatment of a disease or condition of a mammal, for example, a human, and includes, without limitation: (a) preventing the disease or condition from occurring in a subject which may be predisposed to the disease or condition but has not yet been diagnosed as having it; (b) inhibiting the disease or condition, i.e., arresting its development; (c) relieving and or ameliorating the disease or condition, i.e., causing regression of the disease or condition; or (d) curing the disease or condition, i.e., stopping its development or progression. The population of individuals treated by the methods of the invention includes individuals suffering from the undesirable condition or disease, as well as individuals at risk for development of the condition or disease.

“Alkyl” refers to a straight or branched, saturated, aliphatic radical having the number of carbon atoms indicated. Alkyl can include any number of carbons, such as C₁₋₂, C₁₋₃, C₁₋₄, C₁₋₅, C₁₋₆, C₁₋₇, C₁₋₈, C₁₋₉, C₁₋₁₀, C₂₋₃, C₂₋₄, C₂₋₅, C₂₋₆, C₃₋₄, C₃₋₅, C₃₋₆, C₄₋₅, C₄₋₆ and C₅₋₆. For example, C₁₋₆ alkyl includes, but is not limited to, methyl, ethyl, propyl, isopropyl, butyl, isobutyl, sec-butyl, tert-butyl, pentyl, isopentyl, hexyl, etc. Alkyl groups can be substituted or unsubstituted.

“Alkylene” refers to a straight or branched, saturated, aliphatic radical having the number of carbon atoms indicated, and linking at least two other groups, i.e., a divalent hydrocarbon radical. The two moieties linked to the alkylene can be linked to the same atom or different atoms of the alkylene group. For instance, a straight chain alkylene can be the bivalent radical of —(CH₂)ₙ—, where n is 1, 2, 3, 4, 5 or 6. Representative alkylene groups include, but are not limited to, methylene, ethylene, propylene, isopropylene, butylene, isobutylene, sec-butylene, pentylene and hexylene. Alkylene groups can be substituted or unsubstituted.

“Alkenyl” refers to a straight chain or branched hydrocarbon having at least 2 carbon atoms and at least one double bond. Alkenyl can include any number of carbons, such as C₂, C₂₋₃, C₂₋₄, C₂₋₅, C₂₋₆, C₂₋₇, C₂₋₈, C₂₋₉, C₂₋₁₀, C₃, C₃₋₄, C₃₋₅, C₃₋₆, C₄, C₄₋₅, C₄₋₆, C₅, C₅₋₆, and C₆. Alkenyl groups can have any suitable number of double bonds, including, but not limited to, 1, 2, 3, 4, 5 or more. Examples of alkenyl groups include, but are not limited to, vinyl (ethenyl), propenyl, isopropenyl, 1-butenyl, 2-butenyl, isobutenyl, butadienyl, 1-pentenyl, 2-pentenyl, isopentenyl, 1,3-pentadienyl, 1,4-pentadienyl, 1-hexenyl, 2-hexenyl, 3-hexenyl, 1,3-hexadienyl, 1,4-hexadienyl, 1,5-hexadienyl, 2,4-hexadienyl, or 1,3,5-hexatrienyl. Alkenyl groups can be substituted or unsubstituted.

“Alkynyl” refers to either a straight chain or branched hydrocarbon having at least 2 carbon atoms and at least one triple bond. Alkynyl can include any number of carbons, such as C₂, C₂₋₃, C₂₋₄, C₂₋₅, C₂₋₆, C₂₋₇, C₂₋₈, C₂₋₉, C₂₋₁₀, C₃, C₃₋₄, C₃₋₅, C₃₋₆, C₄, C₄₋₅, C₄₋₆, C₅, C₅₋₆, and C₆. Examples of alkynyl groups include, but are not limited to, acetylenyl, propynyl, 1-butynyl, 2-butynyl, isobutynyl, sec-butynyl, butadiynyl, 1-pentynyl, 2-pentynyl, isopentynyl, 1,3-pentadiynyl, 1,4-pentadiynyl, 1-hexynyl, 2-hexynyl, 3-hexynyl, 1,3-hexadiynyl, 1,4-hexadiynyl, 1,5-hexadiynyl, 2,4-hexadiynyl, or 1,3,5-hexatriynyl. Alkynyl groups can be substituted or unsubstituted.

“Halogen” refers to fluorine, chlorine, bromine and iodine.

“Haloalkyl” refers to alkyl, as defined above, where some or all of the hydrogen atoms are replaced with halogen atoms. As for alkyl group, haloalkyl groups can have any suitable number of carbon atoms, such as C₁₋₆. For example, haloalkyl includes trifluoromethyl, flouromethyl, etc. In some instances, the term “perfluoro” can be used to define a compound or radical where all the hydrogens are replaced with fluorine. For example, perfluoromethyl refers to 1,1,1-trifluoromethyl.

“Alkoxy” refers to an alkyl group having an oxygen atom that connects the alkyl group to the point of attachment: alkyl-O—. As for alkyl group, alkoxy groups can have any suitable number of carbon atoms, such as C₁₋₆. Alkoxy groups include, for example, methoxy, ethoxy, propoxy, iso-propoxy, butoxy, 2-butoxy, iso-butoxy, sec-butoxy, tert-butoxy, pentoxy, hexoxy, etc. The alkoxy groups can be further substituted with a variety of substituents. Alkoxy groups can be substituted or unsubstituted.

“Oxo” refers to an oxygen atom that is linked to the remainder of a compound with a double bonded

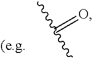 [SMILES: *=O]

wherein the “wavy line” ( [symbol] ) denotes the point of attachment to the remainder of the molecule).

“Oxime” refers to an nitrogen atom that is linked to the remainder of a compound with a double bonded and includes a further covalent bond to a hydroxyl miety

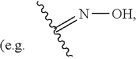 [SMILES: *=NO]

wherein the “wavy line” ( [symbol] ) denotes the point of attachment to the remainder of the molecule).

“Hydroxyalkyl” refers to an alkyl group, as defined above, where at least one of the hydrogen atoms is replaced with a hydroxy group. As for the alkyl group, hydroxyalkyl groups can have any suitable number of carbon atoms, such as C₁₋₆. Exemplary hydroxyalkyl groups include, but are not limited to, hydroxy-methyl, hydroxyethyl (where the hydroxy is in the 1- or 2-position), hydroxypropyl (where the hydroxy is in the 1-, 2- or 3-position), hydroxybutyl (where the hydroxy is in the 1-, 2-, 3- or 4-position), hydroxypentyl (where the hydroxy is in the 1-, 2-, 3-, 4- or 5-position), hydroxyhexyl (where the hydroxy is in the 1-, 2-, 3-, 4-, 5- or 6-position), 1,2-dihydroxyethyl, and the like.

“Aryl” refers to an aromatic ring system having any suitable number of ring atoms and any suitable number of rings. Aryl groups can include any suitable number of ring atoms, such as, 6, 7, 8, 9, 10, 11, 12, 13, 14, 15 or 16 ring atoms, as well as from 6 to 10, 6 to 12, or 6 to 14 ring members. Aryl groups can be monocyclic, fused to form bicyclic or tricyclic groups, or linked by a bond to form a biaryl group. Representative aryl groups include phenyl, naphthyl and biphenyl. Other aryl groups include benzyl, having a methylene linking group. Some aryl groups have from 6 to 12 ring members, such as phenyl, naphthyl or biphenyl. Other aryl groups have from 6 to 10 ring members, such as phenyl or naphthyl. Some other aryl groups have 6 ring members, such as phenyl. Aryl groups can be substituted or unsubstituted.

“Heteroaryl” refers to a monocyclic or fused bicyclic or tricyclic aromatic ring assembly containing 5 to 12 ring atoms, where from 1 to 5 of the ring atoms are a heteroatom such as N, O or S. Additional heteroatoms can also be useful, including, but not limited to, B, Al, Si and P. The heteroatoms can also be oxidized, such as, but not limited to, —S(O)— and —S(O)₂—. Heteroaryl groups can include any number of ring atoms, such as, 3 to 6, 4 to 6, 5 to 6, 3 to 8, 4 to 8, 5 to 8, 6 to 8, 3 to 9, 3 to 10, 3 to 11, or 3 to 12 ring members. Any suitable number of heteroatoms can be included in the heteroaryl groups, such as 1, 2, 3, 4, or 5, or 1 to 2, 1 to 3, 1 to 4, 1 to 5, 2 to 3, 2 to 4, 2 to 5, 3 to 4, or 3 to 5. Heteroaryl groups can have from 5 to 8 ring members and from 1 to 4 heteroatoms, or from 5 to 8 ring members and from 1 to 3 heteroatoms, or from 5 to 6 ring members and from 1 to 4 heteroatoms, or from 5 to 6 ring members and from 1 to 3 heteroatoms. The heteroaryl group can include groups such as pyrrole, pyridine, imidazole, pyrazole, triazole, tetrazole, pyrazine, pyrimidine, pyridazine, triazine (1,2,3-, 1,2,4- and 1,3,5-isomers), thiophene, furan, thiazole, isothiazole, oxazole, and isoxazole. The heteroaryl groups can also be fused to aromatic ring systems, such as a phenyl ring, to form members including, but not limited to, benzopyrroles such as indole and isoindole, benzopyridines such as quinoline and isoquinoline, benzopyrazine (quinoxaline), benzopyrimidine (quinazoline), benzopyridazines such as phthalazine and cinnoline, benzothiophene, and benzofuran. Other heteroaryl groups include heteroaryl rings linked by a bond, such as bipyridine. Heteroaryl groups can be substituted or unsubstituted.

“Heterocycloalkyl” refers to a saturated ring system having from 3 to 12 ring members and from 1 to 4 heteroatoms of N, O and S. Additional heteroatoms can also be useful, including, but not limited to, B, Al, Si and P. The heteroatoms can also be oxidized, such as, but not limited to, —S(O)— and —S(O)₂—. Heterocycloalkyl groups can include any number of ring atoms, such as, 3 to 6, 4 to 6, 5 to 6, 3 to 8, 4 to 8, 5 to 8, 6 to 8, 3 to 9, 3 to 10, 3 to 11, or 3 to 12 ring members. Any suitable number of heteroatoms can be included in the heterocycloalkyl groups, such as 1, 2, 3, or 4, or 1 to 2, 1 to 3, 1 to 4, 2 to 3, 2 to 4, or 3 to 4. The heterocycloalkyl group can include groups such as aziridine, azetidine, pyrrolidine, piperidine, azepane, azocane, quinuclidine, pyrazolidine, imidazolidine, piperazine (1,2-, 1,3- and 1,4-isomers), oxirane, oxetane, tetrahydrofuran, oxane (tetrahydropyran), oxepane, thiirane, thietane, thiolane (tetrahydrothiophene), thiane (tetrahydrothiopyran), oxazolidine, isoxazolidine, thiazolidine, isothiazolidine, dioxolane, dithiolane, morpholine, thiomorpholine, dioxane, or dithiane. The heterocycloalkyl groups can also be fused to aromatic or non-aromatic ring systems to form members including, but not limited to, indoline. Heterocycloalkyl groups can be unsubstituted or substituted. For example, heterocycloalkyl groups can be substituted with C₁₋₆ alkyl or oxo (═O), among many others.

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention

Unless defined otherwise herein, all technical and scientific terms used herein have the same meaning as commonly understood by one of ordinary skill in the art to which this invention pertains.

As used herein, the singular terms “a,” “an,” and “the” include the plural reference unless the context clearly indicates otherwise.

Certain compounds of the present invention possess asymmetric carbon atoms (optical centers) or double bonds; the racemates, diastereomer, geometric isomers, regioisomers and individual isomers (e.g., separate enantiomers) are all intended to be encompassed within the scope of the present invention. In some embodiments, the compounds of the present invention are a particular enantiomer or diastereomer substantially free of other forms.

The term “substantially free” refers to an amount of 10% or less of another form, preferably 8%, 5%, 4%, 3%, 2%, 1%, 0.5%, or less of another form. In some embodiments, the isomer is a stereoisomer.

### III. Compositions of the Invention

Provided herein are cell cultures of expanded hematopoietic stem cells (HSC), cell culture media for maintaining and/or enhancing the expansion of hematopoietic stem cells in culture, and populations of cells containing HSCs made from the methodology described herein. Hematopoietic stem cell can include mammalian and avian hematopoietic stem cells. A population of hematopoietic cells can have the potential for in vivo therapeutic application. The medium includes a base medium or a feed medium as well as a PTEN inhibitor. Any suitable base or feed medium for culturing mammalian cells can be used in the context of the present invention and can include, without limitation, such commercially available media as DMEM medium, IMDM medium, StemSpan Serum-Free Expansion Medium (SFEM), 199/109 medium, HamF10/F12 medium, McCoy's 5A medium, Alpha MEM medium (without and with phenol red), and RPMI 1640 medium. In some embodiments, the base or feed medium is Alpha MEM medium (without phenol red). In some embodiments, the PTEN inhibitor is SF1670 or a chemically altered version thereof.

In some embodiments, the methods, media, systems, and kits provided herein do not include a tetraspanin. In some embodiments, the methods, media, systems, and kits provided herein also includes a retinoic acid receptor (RAR) inhibitor or modulator. In some embodiments, the RAR inhibitor is ER50891.

Populations of cells containing HSCs provided herein confer the same or similar advantages of stem cells found in cord blood. A person of skill in the art would readily recognize the characteristics of stem cells from cord blood and the advantageous properties therein. In some embodiments, at least 10%, 20%, 30%, 40%, 50%, 60%, 70%, 80%, 90%, or 100% of the populations of cells containing HSCs provided herein are expanded HSC cells. In some embodiments, the expanded HSC cells in the populations of cells have retained their stem cell phenotype for an extended period of time. For example, in some embodiments, populations of cells containing HSCs include expanded HSC cells with cell surface phenotypes that include CD45+, CD34+, CD133+, CD90+, CD45RA−, and/or CD38 low/− and have been cultured in vitro for at least 3, 7, 10, 13, 14, 20, 25, 30, 40, or 50 or more days. In some embodiments, populations of cells containing HSCs include expanded HSC cells with cell surface phenotypes that includes CD133+ and/or CD90+ and have been cultured in vitro for at least 3, 7, 10, 13, 14 or more days.

A. Phosphatase and Tensin Homolog (PTEN) Inhibitors

PTEN is known as a tumor suppressor that is mutated in a large number of cancers at high frequency. This protein negatively regulates intracellular levels of phosphatidylinositol-3,4,5-trisphosphate (PIP₃) and functions as a tumor suppressor by negatively regulating Akt/PKB signaling pathway. An inhibitor of PTEN is a compound that decreases, blocks, prevents, or otherwise reduces the natural activity of PTEN.

In some embodiments, the PTEN inhibitor is bpV(phen), bpV(pic), VO—OHpic, bpV(bipy), SF1670, or a chemically altered version of SF1670. In some embodiments, the PTEN inhibitor is SF1670 or a chemically altered version thereof. In some embodiments, the PTEN inhibitor is SF1670.

bpV(phen) is a phosphatase and tensin homolog (PTEN) inhibitor having the structure shown below.

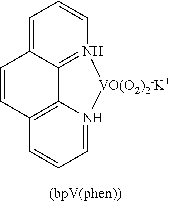

bpV(pic) is a phosphatase and tensin homolog (PTEN) inhibitor having the structure shown below.

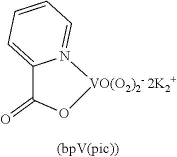

VO—OH(pic) is a phosphatase and tensin homolog (PTEN) inhibitor having the structure shown below.

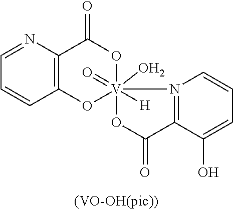

bpV(bipy) is a phosphatase and tensin homolog (PTEN) inhibitor having the structure shown below.

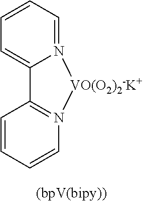

SF1670 is a phosphatase and tensin homolog (PTEN) inhibitor having the structure shown below.

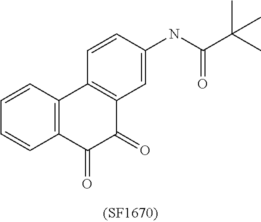 [SMILES: CC(C)(C)C(=O)Nc1ccc2c(c1)C(=O)C(=O)c1ccccc1-2]

A chemically altered SF1670 is a compound that includes one or more structural modifications. Structural modifications can include addition of one or more substituents to SF1670, removal of one or more substituents from the core tri-cyclic structure, and/or replacement of the one or more substituents of the core tri-cyclic structure. Suitable substituents include, but are not limited to oxo, hydroxyl, and C₁₋₈ alkoxy, and C₁₋₈ hydroxyalkyl.

In one aspect, a chemically altered version of SF1670 is a compound of Formula I

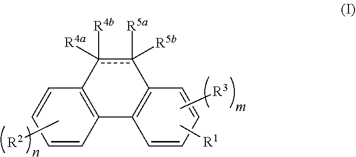

or a pharmaceutically acceptable salt, hydrate, or solvate thereof; wherein
the dashed line (represented by - - - - ) is an optional double bond;
R¹ is selected from the group consisting of —C(O)—NR^{b}—R^{1a}, —NR^{b}—C(O)—R^{1a}, —NR^{b}—C(O)—R^{1b}, —NR^{b}—X—C(O)—R^{1a}, —C(O)—X¹—NR^{b}—R^{1a}, —X¹—C(O)—NR^{b}—R^{1a}, —X¹—NR^{b}—C(O)—R^{1a}, —NR^{b}—C(O)—X¹—C(O)—R^{1b}, —C(O)—NR^{b}—X¹—C(O)—R^{1b}, —NR^{b}—C(O)—O—R^{1a}, —NR^{b}—C(O)—O—R^{1b}, —O—C(O)—NR^{b}—R^{1a}, —X¹—NR^{b}—C(O)—O—R^{1a}, —X¹—O—C(O)—NR^{b}—R^{1a}, —O—R¹, —NR^{b}—R^{1a}, —NR^{b}—C(O)—X¹—O—X¹—R^{1a} and —C(O)—R^{1a};
R^{1a} is selected from the group consisting of H, —C₁₋₁₀ alkyl, —C₁₋₁₀ haloalkyl, and phenyl;
R^{1b} is selected from the group consisting of —OR^{a}, —NR^{a}R^{b},
each R² is independently selected from the group consisting of halogen, —CN, —C₁₋₈ alkyl, —C₂₋₈ alkenyl, —C₂₋₈ alkynyl, —C₁₋₈ haloalkyl, —C₁₋₈ alkoxy, —X¹—C₁₋₈ alkoxy, —C(O)—R^{2a}, —SR^{a}, —X¹—SR^{a}, —OR^{a}, —X¹—OR^{a}, —NR^{a}R^{b}, —X¹—NR^{a}R^{b}, —S(O)₂R^{a}, —S(O)₂NR^{a}R^{b}, —X¹—S(O)₂R^{a}, and —X¹—S(O)₂NR^{a}R^{b};
each R^{2a} is independently selected from the group consisting of H, —C₁₋₁₀ alkyl, —C₁₋₁₀ haloalkyl, —OR^{a}, —X¹—OR^{a}, —NR^{a}R^{b}, and —X¹—NR^{a}R^{b};
each R³ is independently selected from the group consisting of halogen, —CN, —C₁₋₈ alkyl, —C₂₋₈ alkenyl, —C₂₋₈ alkynyl, —C₁₋₈ haloalkyl, —C₁₋₈ alkoxy, —C(O)—R^{3a}, —SR^{a}, —X¹—SR^{a}, —OR^{a}, —X¹—OR^{a}, —NR^{a}R^{b}, —X¹—NR^{a}R^{b}, —S(O)₂R^{a}, —S(O)₂NR^{a}R^{b}, —X¹—S(O)₂R^{a}, and —X¹—S(O)₂NR^{a}R^{b};
each R^{3a} is independently selected from the group consisting of H, —C₁₋₁₀ alkyl, —C₁₋₁₀ haloalkyl, —OR^{a}, —X¹—OR^{a}, —NR^{a}R^{b}, and —X¹—NR^{a}R^{b};
R^{4a} is selected from the group consisting of —OR^{c} and —NR^{c}R^{d}; 1R^{4b} is H or absent; or R^{4a} and R^{4b} are combined to form an oxo or oxime moiety;
R^{5a} is selected from the group consisting of —OR^{c} and —NR^{c}R^{d};
R^{5b} is H or absent; or R^{5a} and R^{5b} are combined to form an oxo or oxime moiety;
   when either R^{4a} and R^{4b} or R^{5a} and R^{5b} combine to form an oxo or oxime moiety, the dashed line is absent;
each R^{a} and R^{b} is independently selected from the group consisting of H and C₁₋₄ alkyl;
each R^{c} and R^{d} is independently selected from the group consisting of H, —C₁₋₈ alkyl, —C₂₋₈ alkenyl, —C₂₋₈ alkynyl, —C₁₋ₛ haloalkyl, —X¹—SR^{a}, —X¹—OR^{a}, —NR^{a}R^{b}, —X¹—NR^{a}R^{b}, —C(O)—H, —C(O)—C₁₋₈alkyl, C₃₋₆ cycloalkyl, heterocycloalkyl, aryl, and heteroaryl;
each X¹ is independently C₁₋₄ alkylene;
the subscript n is an integer from 0 to 3; and
the subscript m is an integer from 0 to 2;
provided that the compound of Formula I is not N-(9,10-Dihydro-9,10-dioxo-2-phenanthrenyl)-2,2-dimethyl-propanamide.

In some aspects, compounds of Formula I can inhibit or alter the activity of PTEN, thereby providing improved conditions for expanding and maintaining hematopoietic stem cells in culture.

In some embodiments, the compound of Formula I has the structure of Formula I-1

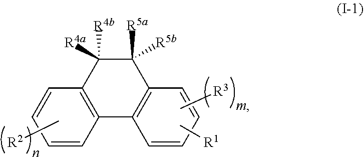

or a pharmaceutically acceptable salt, hydrate, or solvate thereof, wherein
R^{4a} is selected from the group consisting of —OR^{c}, and —NR^{c}R^{d};
R^{4b} is H;
R^{5a} is selected from the group consisting of —OR^{c}, and —NR^{c}R^{d};
R^{5b} is H.

In some embodiments, the compound of Formula I has the structure of Formula I-2

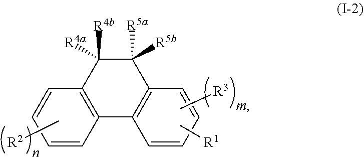

or a pharmaceutically acceptable salt, hydrate, or solvate thereof, wherein
R^{4a} is selected from the group consisting of —OR^{c}, and —NR^{c}R^{d};
R^{4b} is H;
R^{5a} is selected from the group consisting of —OR^{c}, and —NR^{c}R^{d};
R^{5b} is H.

In some embodiments, the compound of Formula I has the structure of Formula I-3

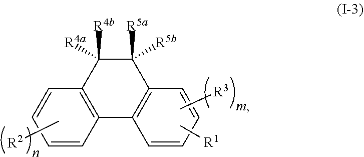

or a pharmaceutically acceptable salt, hydrate, or solvate thereof, wherein
R^{4a} is selected from the group consisting of —OR^{c}, and —NR^{c}R^{d};
R^{4b} is H;
R^{5a} is selected from the group consisting of —OR^{c}, and —NR^{c}R^{d};
R^{5b} is H.

In some embodiments, the compound of Formula I has the structure of Formula I-4

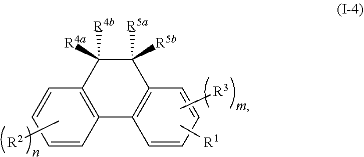

or a pharmaceutically acceptable salt, hydrate, or solvate thereof, wherein
R^{4a} is selected from the group consisting of —OR^{c}, and —NR^{c}R^{d};
R^{4b} is H;
R^{5a} is selected from the group consisting of —OR^{c}, and —NR^{c}R^{d};
R^{5b} is H.

In some embodiments, the compound of Formula I has the structure of Formula I-5

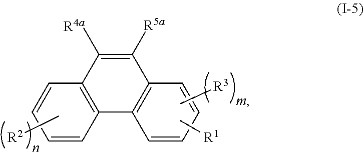

or a pharmaceutically acceptable salt, hydrate, or solvate thereof, wherein
R^{4a} is selected from the group consisting of —OR^{c}, and —NR^{c}R^{d};
R^{5a} is selected from the group consisting of —OR^{c}, and —NR^{c}R^{d}.

In some embodiments, the compound of Formula I has the structure of Formula Ia

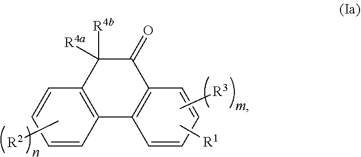

or a pharmaceutically acceptable salt, hydrate, or solvate thereof, wherein
R^{4a} is selected from the group consisting of —OR^{c}, and —NR^{c}R^{d};
R^{4b} is H.

In some embodiments, the compound of Formula Ia has the structure of Formula Ia1

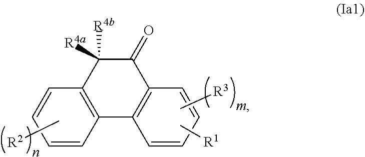

or a pharmaceutically acceptable salt, hydrate, or solvate thereof.

In some embodiments, the compound of Formula Ia has the structure of Formula Ia2

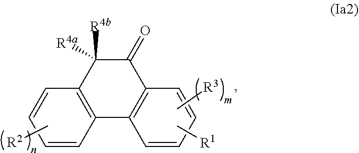

or a pharmaceutically acceptable salt, hydrate, or solvate thereof.

In some embodiments, the compound of Formula I has the structure of Formula Ib

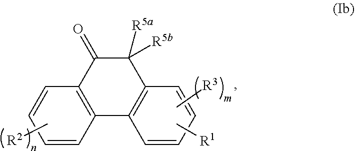

or a pharmaceutically acceptable salt, hydrate, or solvate thereof, wherein
R^{5a} is selected from the group consisting of —OR^{c}, and —NR^{c}R^{d};
R^{5b} is H.

In some embodiments, the compound of Formula Ib has the structure of Formula Ib1

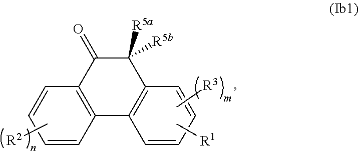

or a pharmaceutically acceptable salt, hydrate, or solvate thereof.

In some embodiments, the compound of Formula Ib has the structure of Formula Ib2

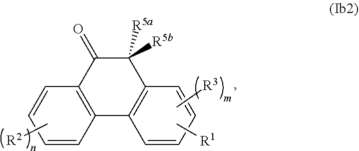

or a pharmaceutically acceptable salt, hydrate, or solvate thereof.

In some embodiments, the compound of Formula I has the structure of Formula II

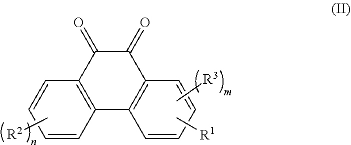

or a pharmaceutically acceptable salt, hydrate, or solvate thereof.

In some embodiments, the compound of Formula II has the structure of Formula IIa

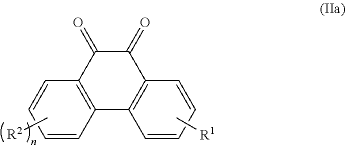

or a pharmaceutically acceptable salt, hydrate, or solvate thereof.

In some embodiments, the compound of Formula II has the structure of Formula IIa1

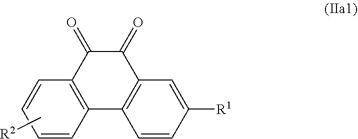

or a pharmaceutically acceptable salt, hydrate, or solvate thereof.

In some embodiments, the compound of Formula II has the structure of Formula IIa2

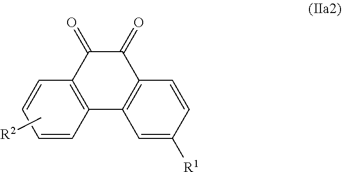

or a pharmaceutically acceptable salt, hydrate, or solvate thereof.

In some embodiments, the compound of Formula II has the structure of Formula IIb1

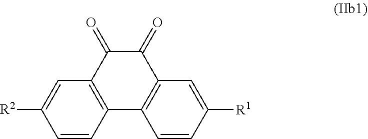

or a pharmaceutically acceptable salt, hydrate, or solvate thereof.

In some embodiments, the compound of Formula II has the structure of Formula Ib2

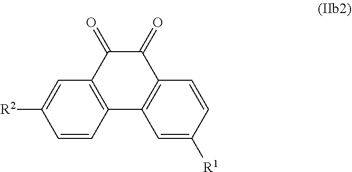

or a pharmaceutically acceptable salt, hydrate, or solvate thereof.

In some embodiments, the compound of Formula I has the structure of Formula III

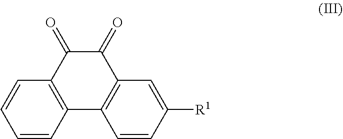

or a pharmaceutically acceptable salt, hydrate, or solvate thereof.

In some embodiments, the compound of Formula I has the structure of Formula IIIa

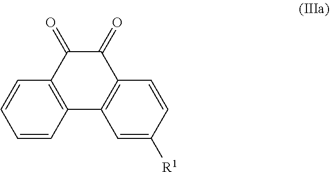

or a pharmaceutically acceptable salt, hydrate, or solvate thereof.

In some embodiments, R¹ in Formulas I, I-1, I-2, I-3, I-4, I-5, Ia, Ia1, Ia2, Ib, Ib1, Ib2, II, IIa, IIa1, IIa2, IIb1, IIb2, III, or IIIa is selected from the group consisting of —C(O)—NR^{b}—R^{1a}, —NR^{b}—C(O)—R^{1a}, —NR^{b}—C(O)—R^{b}, —NR^{b}—X¹—C(O)—R^{1a}, —C(O)—X¹—NR^{b}—R^{1a}, —X¹—C(O)—NR^{b}—R^{1a}, —X¹—NR^{b}—C(O)—R^{1a}, —NR^{b}—C(O)—X¹—C(O)—R^{1b}, —C(O)—NR^{b}—X¹—C(O)—R^{1b}, —NR^{b}—C(O)—O—R^{1a}, —NR^{b}—C(O)—O—R^{1b}, —O—C(O)—NR^{b}—R^{1a}, —X¹—NR^{b}—C(O)—O—R^{1a}, —X¹—O—C(O)—NR^{b}—R^{1a}, —NR^{b}—R^{1a}, and —C(O)R^{1a}.

In some embodiments, R¹ in Formulas I, I-1, I-2, I-3, I-4, I-5, Ia, Ia1, Ia2, Ib, Ib1, Ib2, II, IIa, IIa1, IIa2, IIb1, IIb2, III, or IIIa is selected from the group consisting of —C(O)—NR^{b}—R^{1a}, —NR^{b}—C(O)—R^{1a}, —NR^{b}—X¹—C(O)—R^{1a}, —C(O)—X¹—NR^{b}—R^{1a}, —X¹—C(O)—NR^{b}—R^{1a}, —X¹—NR^{b}—C(O)—R^{1a}, —NR^{b}—C(O)—X¹—C(O)—R^{1b}, —C(O)—NR^{b}—X¹—C(O)—R^{1b}, —NR^{b}—C(O)—O—R^{1a}, —O—C(O)—NR^{b}—R^{1a}, —X¹—NR^{b}—C(O)—O—R^{1a}, and —X¹—O—C(O)—NR^{b}—R^{1a}.

In some embodiments, R¹ in Formulas I, I-1, I-2, I-3, I-4, I-5, Ia, Ia1, Ia2, Ib, Ib1, Ib2, II, IIa, IIa1, IIa2, IIb1, IIb2, III, or IIIa selected from the group consisting of —C(O)—NH—R^{1a}, —NH—C(O)—R^{1a}, —NH—X¹—C(O)—R^{1a}, —C(O)—X¹—NH—R^{1a}, —X¹—C(O)—NH—R^{1a}, —X¹—NH—C(O)—R^{1a}, —NH—C(O)—X¹—C(O)—R^{1b}, —NH—C(O)—O—R^{1a}, and —O—C(O)—NH—R^{1a}.

In some embodiments, R¹ in Formulas I, I-1, I-2, I-3, I-4, I-5, Ia, Ia1, Ia2, Ib, Ib1, Ib2, II, IIa, IIa1, IIa2, IIb1, IIb2, III, or IIIa is selected from the group consisting of —C(O)—NH—R^{1a}, —NH—C(O)—R^{1a}, —NH—X¹—C(O)—R^{1a}, and —NH—C(O)—X¹—C(O)—R^{1b}.

In some embodiments, R¹ in Formulas I, I-1, I-2, I-3, I-4, I-5, Ia, Ia1, Ia2, Ib, Ib1, Ib2, II, IIa, IIa1, IIa2, IIb1, IIb2, III, or IIIa is —NH—C(O)—R^{1a}.

In some embodiments, each R² in Formulas I, I-1, I-2, I-3, I-4, I-5, Ia, Ia1, Ia2, Ib, Ib1, Ib2, II, IIa, IIa1, IIa2, IIb1, or IIb2 is independently selected from the group consisting of halogen, —CN, —C₁₋ₛ alkyl, —C₂₋₈ alkenyl, C₂₋₈ alkynyl, C₁₋₈ haloalkyl, —C₁₋₈ alkoxy, —X¹—C is alkoxy, —C(O)—R^{2a}, —SR^{a}, —X¹—SR^{a}, —NR^{a}R^{b}, —X¹—NR^{a}R^{b}, —S(O)₂R^{a}, —S(O)₂NR^{a}R^{b}, —X¹—S(O)₂R^{a}, and —X¹—S(O)₂NR^{a}R^{b}.

In some embodiments, each R² in Formulas I, I-1, I-2, I-3, I-4, I-5, Ia, Ia1, Ia2, Ib, Ib1, Ib2, II, IIa, IIa1, IIa2, IIb1, or IIb2 is independently selected from the group consisting of halogen, —C₁₋₈ alkyl, C₁₋₈ haloalkyl, —C₁₋₈ alkoxy, —X¹—C₁₋₈ alkoxy, —NR^{a}R^{b}, —X¹—NR^{a}R^{b}, —S(O)₂R^{a}, —S(O)₂NR^{a}R^{b}, —X¹—S(O)₂R^{a}, and —X¹—S(O)₂NR^{a}R^{b}.

In some embodiments, each R² in Formulas I, I-1, I-2, I-3, I-4, I-5, Ia, Ia1, Ia2, Ib, Ib1, Ib2, II, IIa, IIa1, IIa2, IIb1, or IIb2 is independently selected from the group consisting of halogen, —C₁₋₈alkyl, C₁₋₈ haloalkyl, —C₁₋₈ alkoxy, —X¹—C₁₋₈ alkoxy, —NR^{a}R^{b}, and —X¹—NR^{a}R^{b}.

In some embodiments, each R² in Formulas I, I-1, I-2, I-3, I-4, I-5, Ia, Ia1, Ia2, Ib, Ib1, Ib2, II, IIa, IIa1, IIa2, IIb1, or IIb2 is independently selected from the group consisting of halogen, —C₁₋₈ alkyl, C₁₋₈haloalkyl, —NR^{a}R^{b}, and —X¹—NR^{a}R^{b}.

In some embodiments, each R² in Formulas I, I-1, I-2, I-3, I-4, I-5, Ia, Ia1, Ia2, Ib, Ib1, Ib2, II, IIa, IIa1, IIa2, IIb1, or IIb2-NR^{a}R^{b} or —X¹—NR^{a}R^{b}.

In some embodiments, each R³ in Formulas I, I-1, I-2, I-3, I-4, I-5, Ia, Ia1, Ia2, Ib, Ib1, Ib2, or II is independently selected from the group consisting of halogen, —CN, —C₁₋₈ alkyl, —C₂₋₈ alkenyl, C₂₋₈ alkynyl, C₁₋₈ haloalkyl, —C₁₋₈ alkoxy, —X¹—C₁_₈ alkoxy, —C(O)—R^{3a}, —SR^{a}, —X¹—SR^{a}, —NR^{a}R^{b}, —X¹—NR^{a}R^{b}, —S(O)₂R^{a}, —S(O)₂NR^{a}R^{b}, —X¹—S(O)₂R^{a}, and —X¹—S(O)₂NR^{a}R^{b}.

In some embodiments, each R³ in Formulas I, I-1, I-2, I-3, I-4, I-5, Ia, Ia1, Ia2, Ib, Ib1, Ib2, or II is independently selected from the group consisting of halogen, —C₁₋₈ alkyl, C₁₋₈ haloalkyl, —C₁₋₈ alkoxy, —X¹—C₁₋₈ alkoxy, —NR^{a}R^{b}, —X¹—NR^{a}R^{b}, —S(O)₂R^{a}, —S(O)₂NR^{a}R^{b}, —X¹—S(O)₂R^{a}, and —X¹—S(O)₂NR^{a}R^{b}.

In some embodiments, each R³ in Formulas I, I-1, I-2, I-3, I-4, I-5, Ia, Ia1, Ia2, Ib, Ib1, Ib2, or II is independently selected from the group consisting of halogen, —C₁₋₈ alkyl, C₁₋₈ haloalkyl, —C₁₋₈ alkoxy, —X¹—C₁₋₈ alkoxy, —NR^{a}R^{b}, and —X¹—NR^{a}R^{b}.

In some embodiments, each R³ in Formulas I, I-1, I-2, I-3, I-4, I-5, Ia, Ia1, Ia2, Ib, Ib1, Ib2, or II is independently selected from the group consisting of halogen, —C₁₋₈ alkyl, C₁₋₈ haloalkyl, —NR^{a}R^{b}, and —X¹—NR^{a}R^{b}.

In some embodiments, each R³ in Formulas I, I-1, I-2, I-3, I-4, I-5, Ia, Ia1, Ia2, Ib, Ib1, Ib2, or II is —NR^{a}R^{b} or —X¹—NR^{a}R^{b}.

In some embodiments, R^{a} in Formulas I, I-1, I-2, I-3, I-4, I-5, Ia, Ia1, Ia2, Ib, Ib1, Ib2, II, IIa, IIa1, IIa2, IIb1, IIb2, III, or IIIa is C₁₋₁₀ alkyl or C₁₋₁₀ haloalkyl. In some embodiments, R^{a} in Formulas I, I-1, I-2, Ia, Ia1, Ia2, Ib, Ib1, Ib2, II, IIa, IIa1, IIa2, IIb1, IIb2, III, or IIIa is H, C₁₋₁₀ alkyl or C₁₋₁₀ haloalkyl.

In some embodiments, R^{a} in Formulas I, I-1, I-2, I-3, I-4, I-5, Ia, Ia1, Ia2, Ib, Ib1, Ib2, II, IIa, IIa1, IIa2, IIb1, IIb2, III, or IIIa is C₁₋₁₀ alkyl or C₁₋₁₀ haloalkyl. In some embodiments, R^{a} in Formulas I, I-1, I-2, Ia, Ia1, Ia2, Ib, Ib1, Ib2, II, IIa, IIa1, IIa2, IIb1, IIb2, III, or IIIa is C₁₋₁₀ alkyl, C₁₋₁₀ haloalkyl, or phenyl.

In some embodiments, R^{1a} in Formulas I, I-1, I-2, I-3, I-4, I-5, Ia, Ia1, Ia2, Ib, Ib1, Ib2, II, IIa, IIa1, IIa2, IIb1, IIb2, III, or IIIa is C₁₋₁₀ alkyl or C₁₋₁₀ haloalkyl. In some embodiments, R^{1a} in Formulas I, I-1, I-2, Ia, Ia1, Ia2, Ib, Ib1, Ib2, II, IIa, HIa1, IIa2, IIb1, IIb2, III, or IIIa is C₁₋₁₀ alkyl or C₁₋₁₀ haloalkyl.

In some embodiments, R^{a} in Formulas I, I-1, I-2, I-3, I-4, I-5, Ia, Ia1, Ia2, Ib, Ib1, Ib2, II, IIa, IIa1, IIa2, IIb1, IIb2, III, or IIIa is C₁₋₁₀ alkyl or C₁₋₁₀ haloalkyl. In some embodiments, R^{1a} in Formulas I, I-1, I-2, Ia, Ia1, Ia2, Ib, Ib1, Ib2, II, IIa, IIa1, IIa2, IIb1, IIb2, III, or IIIa is C₁₋₆ alkyl or C₁₋₆ haloalkyl.

In some embodiments, R^{1b} in Formulas I, I-1, I-2, I-3, I-4, I-5, Ia, Ia1, Ia2, Ib, Ib1, Ib2, II, IIa, IIa1, IIa2, IIb1, IIb2, III, or IIIa is —OR^{a}. In some embodiments, R^{1b} in Formulas I, I-1, I-2, Ia, Ia1, Ia2, Ib, Ib1, Ib2, II, IIa, IIa1, IIa2, IIb1, IIb2, III, or IIIa is —OH.

In some embodiments, each R^{a} and R^{b} in Formulas I, I-1, I-2, I-3, I-4, I-5, Ia, Ia1, Ia2, Ib, Ib1, Ib2, II, IIa, IIa1, IIa2, IIb1, IIb2, III, or IIIa is independently selected from the group consisting of H and C₁₋₂ alkyl.

In some embodiments, each X¹ in Formulas I, I-1, I-2, I-3, I-4, I-5, Ia, Ia1, Ia2, Ib, Ib1, Ib2, II, IIa, IIa1, IIa2, IIb1, IIb2, III, or IIIa is C₁₋₂ alkylene. In some embodiments, each X¹ in Formulas I, I-1, I-2, Ia, Ia1, Ia2, Ib, Ib1, Ib2, II, IIa, IIa1, IIa2, IIb1, IIb2, III, or IIIa is C₁ alkylene.

In some embodiments, the subscript n in Formulas I, I-1, I-2, I-3, I-4, I-5, Ia, Ia1, Ia2, Ib, Ib1, Ib2, II, or IIa is an integer from 1 to 3. In some embodiments, the subscript n in Formulas I, I-1, I-2, Ia, Ia1, Ia2, Ib, Ib1, Ib2, II, or IIa is 1. In some embodiments, the subscript n in Formulas I, I-1, I-2, Ia, Ia1, Ia2, Ib, Ib1, Ib2, II, or IIa is 0.

In some embodiments, the subscript m in Formulas I, I-1, I-2, I-3, I-4, I-5, Ia, Ia1, Ia2, Ib, Ib1, Ib2, or II is an integer from 1 to 2. In some embodiments, the subscript m in Formulas I, I-1, I-2, Ia, Ia1, Ia2, Ib, Ib1, Ib2, or II is 0. In some embodiments, the subscript m in Formulas I, I-1, I-2, Ia, Ia1, Ia2, Ib, Ib1, Ib2, or II is 1.

In some embodiments, R^{4a} in Formulas I, I-1, I-2, I-3, I-4, I-5, Ia, Ia1, or Ia2 is selected from the group consisting of —OR^{c}. In some embodiments, R^{4a} in Formulas I, I-1, I-2, I-3, I-4, I-5, Ia, Ia1, or Ia2 is selected from the group consisting of —OH, —NH₂, —O—C(O)—CH₃. In some embodiments, R^{4a} in Formulas I, I-1, I-2, I-3, I-4, I-5, Ia, Ia1, or Ia2 is selected from the group consisting of —OH and —NH₂. In some embodiments, R^{4a} in Formulas I, I-1, I-2, Ia, Ia1, or Ia2 is —OH. In some embodiments, R^{4a} in Formulas I, I-1, I-2, Ia, Ia1, or Ia2 is —O—C(O)—CH₃.

In some embodiments, R^{5a} in Formulas I, I-1, I-2, I-3, I-4, I-5, Ia, Ia1, or Ia2 is selected from the group consisting of —OR^{c}. In some embodiments, R^{5a} in Formulas I, I-1, I-2, I-3, I-4, I-5, Ib, Ib1, or Ib2 is selected from the group consisting of —OH, —NH₂, —O—C(O)—CH₃. In some embodiments, R^{5a} in Formulas I, I-1, I-2, I-3, I-4, Ib, Ib, or Ib2 is selected from the group consisting of —OH and —NH₂. In some embodiments, R^{5a} in Formulas I, I-1, I-2, Ib, Ib, or Ib2 is —OH. In some embodiments, R^{5a} in Formulas I, I-1, I-2, Ia, Ia1, or Ia2 is —O—C(O)—CH₃.

In some embodiments, each R^{c} and R^{d} in Formulas I, I-1, I-2, I-3, I-4, I-5, Ib, Ib1, or Ib2 is independently selected from the group consisting of H, C₁₋₈ alkyl, —C₂₋₈ alkenyl, C₂₋₈ alkynyl, C₁₋₈ haloalkyl, —C₁₋₈ alkoxy, —X¹—C₁₋₈ alkoxy, —SR^{a}, —X¹—SR^{a}, —OR^{a}, —X¹—OR^{a}, —NR^{a}R^{b}, —X¹⁻NR^{a}R^{b}, —C(O)—H, and —C(O)—C₁₋₈alkyl. In some embodiments, each R^{c} and R^{d} in Formulas I, I-1, I-2, I-3, I-4, I-5, Ib, Ib1, or Ib2 is independently selected from the group consisting of H, C₁₋₄ alkyl, —C(O)—H, —C(O)—C₁₋₄alkyl.

In some embodiments, the compound of Formula I has the structure of Formula II

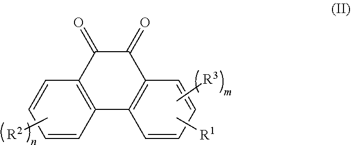

or a pharmaceutically acceptable salt, hydrate, or solvate thereof; wherein,
R¹ is selected from the group consisting of —C(O)—NH—R^{a}; —NH—C(O)—R^{1a}; —NH—X¹—C(O)—R^{1a}; —C(O)—X¹—NH—R^{1a}; —NH—C(O)—X¹—C(O)—R^{1b}; —X¹—C(O)—NH—R^{a}; —X¹—NH—C(O)—R^{1a}; —NH—C(O)—X¹—C(O)—R^{1b}; —NH—R^{1a}; and —C(O)—R^{1a};
each R² is independently selected from the group consisting of halogen, —CN, —C₁₋₈ alkyl, —C₂₋₈ alkenyl, C₂₋₈ alkynyl, C₁₋₈ haloalkyl, —C₁₋₈ alkoxy, —X¹—C₁₋₈ alkoxy, —C(O)—R^{2a}, —SR^{a}, —X¹—SR^{a}, —NR^{a}R^{b}, —Xi-NR^{a}R^{b}, —S(O)₂R^{a}, —S(O)₂NR^{a}R^{b}, —X¹—S(O)₂R^{a}, and —X¹—S(O)₂NR^{a}R^{b}
each R³ is independently selected from the group consisting of halogen, —CN, —C₁₋₈ alkyl, —C₂₋₈ alkenyl, C₂₋₈ alkynyl, C₁₋₈ haloalkyl, —C₁₋₈ alkoxy, —X¹—C₁₋₈alkoxy, —C(O)—R^{3a}, —SR^{a}, —X¹—SR^{a}, —NR^{a}R^{b}, —X¹—NR^{a}R^{b}, —S(O)₂R^{a}, —S(O)₂NR^{a}R^{b}, —X¹—S(O)₂R^{a}, and —X¹—S(O)₂NR^{a}R^{b}
R^{1a} is selected from the group consisting of H, C₁₋₁₀ alkyl; C₁₋₁₀ haloalkyl;
R^{1b} is selected from the group consisting of —OR^{a}, —NR^{a}R^{b},
each R^{2a} and R^{3a} is independently selected from the group consisting of H, C₁₋₁₀ alkyl, C₁₋₁₀ haloalkyl, —OR^{a}, —X¹—OR^{a}, —NR^{a}R^{b}, and —X¹—NR^{a}R^{b};
each R^{a} and R^{b} is independently selected from the group consisting of H and C₁₋₄ alkyl;
each X¹ is C₁₋₄ alkylene;
the subscript n is an integer from 0 to 3; and
the subscript m is an integer from 0 to 2;
provided that the compound of Formula II is not N-(9,10-Dihydro-9,10-dioxo-2-phenanthrenyl)-2,2-dimethyl-propanamide

In some embodiments, the compound of Formula II has the structure of Formula IIa

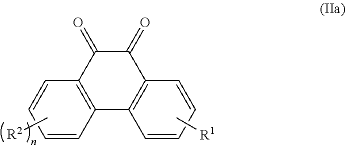

or a pharmaceutically acceptable salt, hydrate, or solvate thereof; wherein,
R¹ is selected from the group consisting of —C(O)—NH—R^{a}; —NH—C(O)—R^{1a}; —NH—X¹—C(O)—Rla; and —NH—C(O)—X¹—C(O)—Rlb
R² is —NH₂;
R^{1a} is selected from the group consisting of C₁₋₁₀ alkyl; and C₁₋₁₀ haloalkyl;
R^{1b} is OH;
R^{a} and R^{b} are independently selected from the group consisting of H and C₁₋₄ alkyl;
X¹ is C₁₋₂ alkylene; and
the subscript n is an integer from 0 to 1;
provided that the compound of Formula II is not N-(9,10-Dihydro-9,10-dioxo-2-phenanthrenyl)-2,2-dimethyl-propanamide.

In some embodiments, the compound of Formula I has the structure of Formula I-5

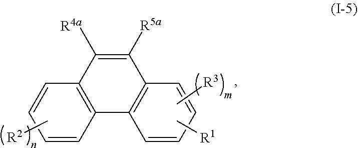

or a pharmaceutically acceptable salt, hydrate, or solvate thereof; wherein,
R¹ is selected from the group consisting of —C(O)—NH—R^{a}; —NH—C(O)—R^{1a}; —NH—X¹—C(O)—R^{1a}; and —NH—C(O)—X¹—C(O)—R^{b};
each R² is —NH₂;
R^{1a} is selected from the group consisting of C₁₋₁₀ alkyl; and C₁₋₁₀ haloalkyl;
R^{1b} is OH;
R^{a} and R^{b} are independently selected from the group consisting of H and C₁₋₄ alkyl;
R^{4a} is selected from the group consisting of —OR^{c} and —NR^{c}R^{d};
R^{5a} is selected from the group consisting of —OR^{c} and —NR^{c}R^{d};
each R^{c} and R^{d} is independently selected from the group consisting of H, C₁₋₄ alkyl, —C(O)—H, —C(O)—C₁₋₄alkyl;
X¹ is C₁₋₂ alkylene; and
the subscript n is an integer from 0 to 3.

In some embodiments, the chemically altered version of SF1670 is a selected from Table 1.

TABLE 1
Particular Compounds of Formula (I)

| Compound | Structure |
|---|---|
| 1.001 | 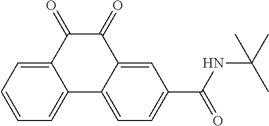 [SMILES: CC(C)(C)NC(=O)c1ccc2c(c1)C(=O)C(=O)c1ccccc1-2] |
| 1.002 | 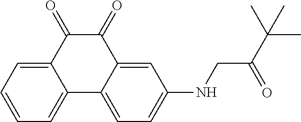 [SMILES: CC(C)(C)C(=O)CNc1ccc2c(c1)C(=O)C(=O)c1ccccc1-2] |
| 1.003 | 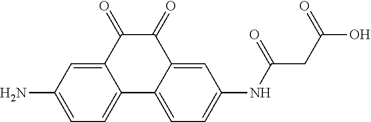 [SMILES: Nc1ccc2c(c1)C(=O)C(=O)c1cc(NC(=O)CC(=O)O)ccc1-2] |
| 1.004 | 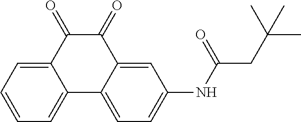 [SMILES: CC(C)(C)CC(=O)Nc1ccc2c(c1)C(=O)C(=O)c1ccccc1-2] |
| 1.005 | 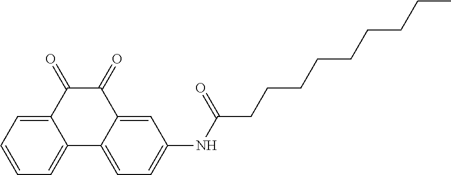 [SMILES: CCCCCCCCCC(=O)Nc1ccc2c(c1)C(=O)C(=O)c1ccccc1-2] |
| 1.006 | 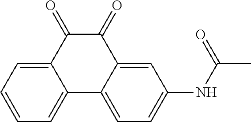 [SMILES: CC(=O)Nc1ccc2c(c1)C(=O)C(=O)c1ccccc1-2] |
| 1.007 | 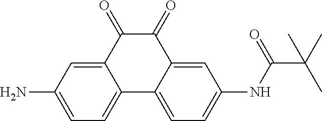 [SMILES: CC(C)(C)C(=O)Nc1ccc2c(c1)C(=O)C(=O)c1cc(N)ccc1-2] |
| 1.008 | 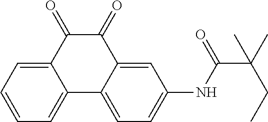 [SMILES: CCC(C)(C)C(=O)Nc1ccc2c(c1)C(=O)C(=O)c1ccccc1-2] |
| 1.009 | 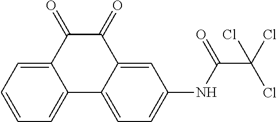 [SMILES: O=C1C(=O)c2cc(NC(=O)C(Cl)(Cl)Cl)ccc2-c2ccccc21] |
| 1.010 | 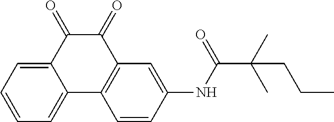 [SMILES: CCCC(C)(C)C(=O)Nc1ccc2c(c1)C(=O)C(=O)c1ccccc1-2] |
| 1.011 | 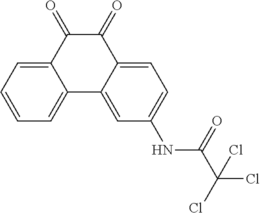 [SMILES: O=C1C(=O)c2ccc(NC(=O)C(Cl)(Cl)Cl)cc2-c2ccccc21] |
| 1.012 | 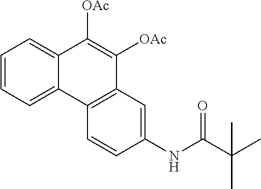 [SMILES: CC(=O)Oc1c(OC(C)=O)c2cc(NC(=O)C(C)(C)C)ccc2c2ccccc12] |
| 1.013 | 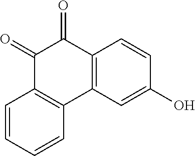 [SMILES: O=C1C(=O)c2ccc(O)cc2-c2ccccc21] |
| 1.014 | 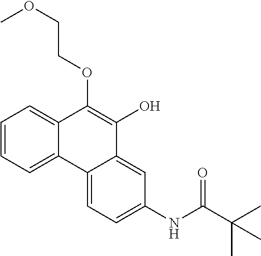 [SMILES: COCCOc1c(O)c2cc(NC(=O)C(C)(C)C)ccc2c2ccccc12] |
| 1.015 | 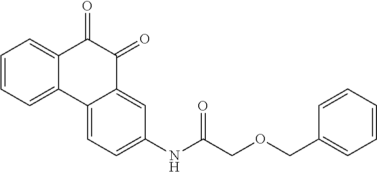 [SMILES: O=C(COCc1ccccc1)Nc1ccc2c(c1)C(=O)C(=O)c1ccccc1-2] |
| 1.016 | 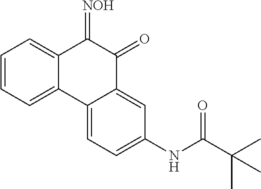 |
| 1.017 | |

The cell culture media compositions for use in the methods of the present invention can include about 10-6000 nM PTEN inhibitor, such as about 50-450 nM, 100-400 nM, about 150-350 nM, about 200-300 nM, about 225-275 nM, or about 240-260 nM, such as about 300-3000 nM, 500-2000 nM, about 550-1550 nM, about 800-1200 nM, about 900-1100 nM, or about 950-1050 nM, or such as any of about 10 nM, 15 nM, 20 nM, 25 nM, 30 nM, 35 nM, 40 nM, 45 nM, 50 nM, 55 nM, 60 nM, 65 nM, 70 nM 75 nM, 80 nM, 85 nM, 90 nM, 95 nM, 100 nM, 105 nM, 110 nM, 115 nM, 120 nM, 125 nM, 130 nM, 135 nM, 140 nM, 145 nM, 150 nM, 155 nM, 160 nM, 165 nM, 170 nM, 175 nM, 180 nM, 185 nM, 190 nM, 195 nM, 200 nM, 205 nM, 210 nM, 215 nM, 220 nM, 225 nM, 230 nM, 240 nM, 245 nM, 250 nM, 255 nM, 260 nM, 265 nM, 270 nM, 275 nM, 280 nM, 285 nM, 290 nM, 295 nM, 300 nM, 325 nM, 350 nM, 400 nM, 425 nM, 450 nM, 475 nM, 500 nM, 525 nM, 550 nM, 575 nM, 600 nM, 625 nM, 650 nM, 675 nM, 700 nM, 725 nM, 750 nM, 775 nM, 800 nM, 825 nM, 850 nM, 875 nM, 900 nM, 925 nM, 950 nM, 975 nM, 1000 nM, 1100 nM, 1200 nM, 1300 nM, 1400 nM, 1500 nM, 1600 nM, 1700 nM, 1800 nM, 1900 nM, 2000 nM, 2100 nM, 2200 nM, 2300 nM, 2400 nM, 2500 nM, 2600 nM, 2700 nM, 2800 nM, 2900 nM, 3000 nM, 3100 nM, 3200 nM, 3300 nM, 3400 nM, 3500 nM, 3600 nM, 3700 nM, 3800 nM, 3900 nM, 4000 nM, 5000 nM, 6000 nM or more of PTEN inhibitor, including values falling in between these concentrations. In some embodiments, cell culture media compositions for use in the methods of the present invention can include about 500 nM of PTEN inhibitor.

### Preparation of Compounds

Certain compounds of the invention can be prepared following methodology as described in the Examples section of this document. In addition, the syntheses of certain intermediate compounds that are useful in the preparation of compounds of the invention are also described.

B. Cytokines and Growth Factors

The cell culture media (e.g. base media or feed media) for use in the methods disclosed herein can contain one or more cytokines or growth factors. These agents promote the survival, maintenance, expansion, or enhancement of HSCs and can be procured via commercially available sources.

Cell culture media for culturing HSCs can include thrombopoietin (TPO). Thrombopoietin is a glycoprotein hormone produced by the liver and kidney which regulates the production of platelets. It stimulates the production and differentiation of megakaryocytes, the bone marrow cells that bud off large numbers of platelets. The cell culture media compositions for use in the methods of the present invention can include about 50-250 ng/mL of TPO such as about 75-225 ng/mL, about 100-200 ng/mL, or about 125-175 ng/mL, or such as any of about 75 ng/mL, 80 ng/mL, 85 ng/mL, 90 ng/mL, 95 ng/mL, 100 ng/mL, 105 ng/mL, 110 ng/mL, 115 ng/mL, 120 ng/mL, 125 ng/mL, 130 ng/mL, 135 ng/mL, 140 ng/mL, 141 ng/mL, 142 ng/mL, 143 ng/mL, 144 ng/mL, 145 ng/mL, 146 ng/mL, 147 ng/mL, 148 ng/mL, 149 ng/mL, 150 ng/mL, 151 ng/mL, 152 ng/mL, 153 ng/mL, 154 ng/mL, 155 ng/mL, 156 ng/mL, 157 ng/mL, 158 ng/mL, 159 ng/mL, 160 ng/mL, 165 ng/mL, 170 ng/mL, 175 ng/mL, 180 ng/mL, 185 ng/mL, 190 ng/mL, 195 ng/mL, 200 ng/mL, 205 ng/mL, 210 ng/mL, 215 ng/mL, 220 ng/mL, 225 ng/mL, 230 ng/mL, 235 ng/mL, 240 ng/mL, 245 ng/mL, or 250 ng/mL or more TPO, including values falling in between these concentrations. In some embodiments, the concentration of TPO in the media is about 150 ng/mL.

Any of the cell culture media disclosed herein can also include stem cell factor (also known as SCF, KIT-ligand, KL, or steel factor). SCF is a cytokine that binds to the c-KIT receptor (CD117) and which plays a role in the regulation of HSCs in bone marrow. SCF has been shown to increase the survival of HSCs in vitro and contributes to the self-renewal and maintenance of HSCs in-vivo. The cell culture media compositions for use in the methods of the present invention can include about 5-100 ng/mL of SCF, such as about 10-90 ng/mL, about 20-80, ng/mL about 30-70 ng/mL, about 40-60 ng/mL, or about 45-55 ng/mL, or such as any of about 5 ng/mL, 10 ng/mL, 15 ng/mL, 20 ng/mL, 25 ng/mL, 30 ng/mL, 35 ng/mL, 40 ng/mL, 41 ng/mL, 42 ng/mL, 43 ng/mL, 44 ng/mL, 45 ng/mL, 46 ng/mL, 47 ng/mL, 48 ng/mL, 49 ng/mL, 50 ng/mL, 51 ng/mL, 52 ng/mL, 53 ng/mL, 54 ng/mL, 55 ng/mL, 56 ng/mL, 57 ng/mL, 58 ng/mL, 59 ng/mL, 60 ng/mL, 65 ng/mL, 70 ng/mL, 75 ng/mL, 80 ng/mL, 85 ng/mL, 90 ng/mL, 95 ng/mL, 100 ng/mL or more SCF, including values falling in between these concentrations. In some embodiments, the cell culture media compositions for use in the methods of the present invention can include concentrations at 100 ng/mL or above. Accordingly, concentrations of SCF also include 110 ng/mL, 115 ng/mL, 120 ng/mL, 125 ng/mL, 130 ng/mL, 135 ng/mL, 140 ng/mL, 145 ng/mL, 150 ng/mL, 155 ng/mL 160 ng/mL, 165 ng/mL, 170 ng/mL, 175 ng/mL, 180 ng/mL 185 ng/mL, 190 ng/mL, 200 ng/mL, or more SCF, including values falling in between these concentrations. In some embodiments, the concentration of SCF in the media is about 100 ng/mL.

The cell culture media disclosed herein can also contain insulin-like growth factor 1 (IGF-1; also called somatomedin C). IGF-1 is a hormone similar in molecular structure to insulin. It plays an important role in childhood growth and has anabolic effects in adults. The cell culture media compositions for use in the methods of the present invention can include about 100-400 ng/mL IGF-1, such as about 125-375 ng/mL, about 150-350 ng/mL, about 175-325 ng/mL, about 200-300 ng/mL, about 225-275 ng/mL, about 240-260 ng/mL, or about 245-255 ng/mL, or such as any of about 100 ng/mL, 105 ng/mL, 110 ng/mL, 115 ng/mL, 120 ng/mL, 125 ng/mL, 130 ng/mL, 135 ng/mL, 140 ng/mL, 145 ng/mL, 150 ng/mL, 155 ng/mL, 160 ng/mL, 165 ng/mL, 170 ng/mL, 175 ng/mL, 180 ng/mL, 185 ng/mL, 190 ng/mL, 195 ng/mL, 200 ng/mL, 205 ng/mL, 210 ng/mL, 215 ng/mL, 220 ng/mL, 225 ng/mL, 230 ng/mL, 235 ng/mL, 240 ng/mL, 241 ng/mL, 242 ng/mL, 243 ng/mL, 244 ng/mL, 245 ng/mL, 246 ng/mL, 247 ng/mL, 248 ng/mL, 249 ng/mL, 250 ng/mL, 251 ng/mL, 252 ng/mL, 253 ng/mL, 254 ng/mL, 255 ng/mL, 256 ng/mL, 257 ng/mL, 258 ng/mL, 259 ng/mL, 260 ng/mL, 265 ng/mL, 270 ng/mL, 275 ng/mL, 280 ng/mL, 285 ng/mL, 290 ng/mL, 295 ng/mL, 300 ng/mL, 305 ng/mL, 310 ng/mL, 315 ng/mL, 320 ng/mL, 325 ng/mL, 330 ng/mL, 335 ng/mL, 340 ng/mL, 345 ng/mL, 350 ng/mL, 355 ng/mL, 360 ng/mL, 365 ng/mL, 370 ng/mL, 375 ng/mL, 380 ng/mL, 385 ng/mL, 390 ng/mL, 395 ng/mL, or 400 ng/mL or more IGF-1, including values falling in between these concentrations. In some embodiments, the concentration of IGF-1 is the media is about 250 ng/mL

The cell culture media for culturing HSCs provided herein can further include fms-related tyrosine kinase 3 ligand (FLT3L). FLT3L is a cytokine that stimulates cell growth, proliferation, and differentiation. The cell culture media compositions for use in the methods of the present invention can include about 20-400 ng/mL FLT3L, such as about 40-375 ng/mL, about 60-350 ng/mL, about 80-325 ng/mL, about 100-300 ng/mL, about 140-275 ng/mL, about 160-260 ng/mL, or about 180-255 ng/mL, or such as any of about 20 ng/mL, 40 ng/mL, 60 ng/mL, 80 ng/mL, 100 ng/mL, 105 ng/mL, 110 ng/mL, 115 ng/mL, 120 ng/mL, 125 ng/mL, 130 ng/mL, 135 ng/mL, 140 ng/mL, 145 ng/mL, 150 ng/mL, 155 ng/mL, 160 ng/mL, 165 ng/mL, 170 ng/mL, 175 ng/mL, 180 ng/mL, 185 ng/mL, 190 ng/mL, 195 ng/mL, 200 ng/mL, 205 ng/mL, 210 ng/mL, 215 ng/mL, 220 ng/mL, 225 ng/mL, 230 ng/mL, 235 ng/mL, 240 ng/mL, 241 ng/mL, 242 ng/mL, 243 ng/mL, 244 ng/mL, 245 ng/mL, 246 ng/mL, 247 ng/mL, 248 ng/mL, 249 ng/mL, 250 ng/mL, 251 ng/mL, 252 ng/mL, 253 ng/mL, 254 ng/mL, 255 ng/mL, 256 ng/mL, 257 ng/mL, 258 ng/mL, 259 ng/mL, 260 ng/mL, 265 ng/mL, 270 ng/mL, 275 ng/mL, 280 ng/mL, 285 ng/mL, 290 ng/mL, 295 ng/mL, 300 ng/mL, 305 ng/mL, 310 ng/mL, 315 ng/mL, 320 ng/mL, 325 ng/mL, 330 ng/mL, 335 ng/mL, 340 ng/mL, 345 ng/mL, 350 ng/mL, 355 ng/mL, 360 ng/mL, 365 ng/mL, 370 ng/mL, 375 ng/mL, 380 ng/mL, 385 ng/mL, 390 ng/mL, 395 ng/mL, or 400 ng/mL or more FLT3L, including values falling in between these concentrations. In some embodiments, the concentration of FLT3L in the media is about 100 ng/mL.

The cell culture media for culturing HSCs provided herein can further include human growth hormone (HGH). HGH is a protein hormone that stimulates cell growth, proliferation, and differentiation. The cell culture media compositions for use in the methods of the present invention can include about 100-400 ng/mL EGF, such as about 125-375 ng/mL, about 150-350 ng/mL, about 175-325 ng/mL, about 200-300 ng/mL, about 225-275 ng/mL, about 240-260 ng/mL, or about 245-255 ng/mL, or such as any of about 100 ng/mL, 105 ng/mL, 110 ng/mL, 115 ng/mL, 120 ng/mL, 125 ng/mL, 130 ng/mL, 135 ng/mL, 140 ng/mL, 145 ng/mL, 150 ng/mL, 155 ng/mL, 160 ng/mL, 165 ng/mL, 170 ng/mL, 175 ng/mL, 180 ng/mL, 185 ng/mL, 190 ng/mL, 195 ng/mL, 200 ng/mL, 205 ng/mL, 210 ng/mL, 215 ng/mL, 220 ng/mL, 225 ng/mL, 230 ng/mL, 235 ng/mL, 240 ng/mL, 241 ng/mL, 242 ng/mL, 243 ng/mL, 244 ng/mL, 245 ng/mL, 246 ng/mL, 247 ng/mL, 248 ng/mL, 249 ng/mL, 250 ng/mL, 251 ng/mL, 252 ng/mL, 253 ng/mL, 254 ng/mL, 255 ng/mL, 256 ng/mL, 257 ng/mL, 258 ng/mL, 259 ng/mL, 260 ng/mL, 265 ng/mL, 270 ng/mL, 275 ng/mL, 280 ng/mL, 285 ng/mL, 290 ng/mL, 295 ng/mL, 300 ng/mL, 305 ng/mL, 310 ng/mL, 315 ng/mL, 320 ng/mL, 325 ng/mL, 330 ng/mL, 335 ng/mL, 340 ng/mL, 345 ng/mL, 350 ng/mL, 355 ng/mL, 360 ng/mL, 365 ng/mL, 370 ng/mL, 375 ng/mL, 380 ng/mL, 385 ng/mL, 390 ng/mL, 395 ng/mL, or 400 ng/mL or more EGF, including values falling in between these concentrations. In some embodiments, the concentration of HGH in the media is about 250 ng/mL.

The cell culture media for culturing HSCs provided herein can further include epidermal growth factor (EGF). EGF is a growth factor that stimulates cell growth, proliferation, and differentiation by binding to its receptor EGFR. The cell culture media compositions for use in the methods of the present invention can include about 100-400 ng/mL EGF, such as about 125-375 ng/mL, about 150-350 ng/mL, about 175-325 ng/mL, about 200-300 ng/mL, about 225-275 ng/mL, about 240-260 ng/mL, or about 245-255 ng/mL, or such as any of about 100 ng/mL, 105 ng/mL, 110 ng/mL, 115 ng/mL, 120 ng/mL, 125 ng/mL, 130 ng/mL, 135 ng/mL, 140 ng/mL, 145 ng/mL, 150 ng/mL, 155 ng/mL, 160 ng/mL, 165 ng/mL, 170 ng/mL, 175 ng/mL, 180 ng/mL, 185 ng/mL, 190 ng/mL, 195 ng/mL, 200 ng/mL, 205 ng/mL, 210 ng/mL, 215 ng/mL, 220 ng/mL, 225 ng/mL, 230 ng/mL, 235 ng/mL, 240 ng/mL, 241 ng/mL, 242 ng/mL, 243 ng/mL, 244 ng/mL, 245 ng/mL, 246 ng/mL, 247 ng/mL, 248 ng/mL, 249 ng/mL, 250 ng/mL, 251 ng/mL, 252 ng/mL, 253 ng/mL, 254 ng/mL, 255 ng/mL, 256 ng/mL, 257 ng/mL, 258 ng/mL, 259 ng/mL, 260 ng/mL, 265 ng/mL, 270 ng/mL, 275 ng/mL, 280 ng/mL, 285 ng/mL, 290 ng/mL, 295 ng/mL, 300 ng/mL, 305 ng/mL, 310 ng/mL, 315 ng/mL, 320 ng/mL, 325 ng/mL, 330 ng/mL, 335 ng/mL, 340 ng/mL, 345 ng/mL, 350 ng/mL, 355 ng/mL, 360 ng/mL, 365 ng/mL, 370 ng/mL, 375 ng/mL, 380 ng/mL, 385 ng/mL, 390 ng/mL, 395 ng/mL, or 400 ng/mL or more EGF, including values falling in between these concentrations.

Any of the cell culture media disclosed herein can also include hepatocyte growth factor (HGF). HGF is a paracrine cellular growth, motility and morphogenic factor. It is secreted by mesenchymal cells and acts primarily upon epithelial cells and endothelial cells, but also acts on hematopoietic progenitor cells and T cells. HGF has been shown to have a major role in embryonic organ development, specifically in myogenesis, in adult organ regeneration and in wound healing. The cell culture media compositions for use in the methods of the present invention can include about 100-400 ng/mL HGF, such as about 125-375 ng/mL, about 150-350 ng/mL, about 175-325 ng/mL, about 200-300 ng/mL, about 225-275 ng/mL, about 240-260 ng/mL, or about 245-255 ng/mL, or such as any of about 100 ng/mL, 105 ng/mL, 110 ng/mL, 115 ng/mL, 120 ng/mL, 125 ng/mL, 130 ng/mL, 135 ng/mL, 140 ng/mL, 145 ng/mL, 150 ng/mL, 155 ng/mL, 160 ng/mL, 165 ng/mL, 170 ng/mL, 175 ng/mL, 180 ng/mL, 185 ng/mL, 190 ng/mL, 195 ng/mL, 200 ng/mL, 205 ng/mL, 210 ng/mL, 215 ng/mL, 220 ng/mL, 225 ng/mL, 230 ng/mL, 235 ng/mL, 240 ng/mL, 241 ng/mL, 242 ng/mL, 243 ng/mL, 244 ng/mL, 245 ng/mL, 246 ng/mL, 247 ng/mL, 248 ng/mL, 249 ng/mL, 250 ng/mL, 251 ng/mL, 252 ng/mL, 253 ng/mL, 254 ng/mL, 255 ng/mL, 256 ng/mL, 257 ng/mL, 258 ng/mL, 259 ng/mL, 260 ng/mL, 265 ng/mL, 270 ng/mL, 275 ng/mL, 280 ng/mL, 285 ng/mL, 290 ng/mL, 295 ng/mL, 300 ng/mL, 305 ng/mL, 310 ng/mL, 315 ng/mL, 320 ng/mL, 325 ng/mL, 330 ng/mL, 335 ng/mL, 340 ng/mL, 345 ng/mL, 350 ng/mL, 355 ng/mL, 360 ng/mL, 365 ng/mL, 370 ng/mL, 375 ng/mL, 380 ng/mL, 385 ng/mL, 390 ng/mL, 395 ng/mL, or 400 ng/mL or more HGF, including values falling in between these concentrations.

The cell culture media disclosed herein can also contain pleiotrophin (PTN). PTN is a developmentally regulated protein that has been shown to be involved in tumor growth and metastasis presumably by activating tumor angiogenesis. The cell culture media compositions for use in the methods of the present invention can include about 100-400 ng/mL PTN, such as about 125-375 ng/mL, about 150-350 ng/mL, about 175-325 ng/mL, about 200-300 ng/mL, about 225-275 ng/mL, about 240-260 ng/mL, or about 245-255 ng/mL, or such as any of about 100 ng/mL, 105 ng/mL, 110 ng/mL, 115 ng/mL, 120 ng/mL, 125 ng/mL, 130 ng/mL, 135 ng/mL, 140 ng/mL, 145 ng/mL, 150 ng/mL, 155 ng/mL, 160 ng/mL, 165 ng/mL, 170 ng/mL, 175 ng/mL, 180 ng/mL, 185 ng/mL, 190 ng/mL, 195 ng/mL, 200 ng/mL, 205 ng/mL, 210 ng/mL, 215 ng/mL, 220 ng/mL, 225 ng/mL, 230 ng/mL, 235 ng/mL, 240 ng/mL, 241 ng/mL, 242 ng/mL, 243 ng/mL, 244 ng/mL, 245 ng/mL, 246 ng/mL, 247 ng/mL, 248 ng/mL, 249 ng/mL, 250 ng/mL, 251 ng/mL, 252 ng/mL, 253 ng/mL, 254 ng/mL, 255 ng/mL, 256 ng/mL, 257 ng/mL, 258 ng/mL, 259 ng/mL, 260 ng/mL, 265 ng/mL, 270 ng/mL, 275 ng/mL, 280 ng/mL, 285 ng/mL, 290 ng/mL, 295 ng/mL, 300 ng/mL, 305 ng/mL, 310 ng/mL, 315 ng/mL, 320 ng/mL, 325 ng/mL, 330 ng/mL, 335 ng/mL, 340 ng/mL, 345 ng/mL, 350 ng/mL, 355 ng/mL, 360 ng/mL, 365 ng/mL, 370 ng/mL, 375 ng/mL, 380 ng/mL, 385 ng/mL, 390 ng/mL, 395 ng/mL, or 400 ng/mL or more PTN, including values falling in between these concentrations. In some embodiments, PTN does not improve the maintenance or enhancement of hematopoietic stem cells.

In further embodiments, the cell culture media compositions disclosed herein can additionally contain basic fibroblast growth factor (bFGF, FGF2 or FGF-β). bFGF is a critical component of human embryonic stem cell culture medium. However, while the growth factor is necessary for the cells to remain in an undifferentiated state, the mechanisms by which it does this are poorly defined. The cell culture media compositions for use in the methods of the present invention can include about 25-225 ng/mL of bFGF such as about 50-200 ng/mL, about 100-200 ng/mL, about 100-150 ng/mL, or about 115-135 ng/mL, or such as any of about 75 ng/mL, 80 ng/mL, 85 ng/mL, 90 ng/mL, 95 ng/mL, 100 ng/mL, 105 ng/mL, 110 ng/mL, 115 ng/mL, 116 ng/mL, 117 ng/mL, 118 ng/mL, 119 ng/mL, 120 ng/mL, 121 ng/mL, 122 ng/mL, 123 ng/mL, 124 ng/mL, 125 ng/mL, 126 ng/mL, 127 ng/mL, 128 ng/mL, 129 ng/mL, 130 ng/mL, 131 ng/mL, 132 ng/mL, 133 ng/mL, 134 ng/mL, 135 ng/mL, 140 ng/mL, 141 ng/mL, 142 ng/mL, 143 ng/mL, 144 ng/mL, 145 ng/mL, 146 ng/mL, 147 ng/mL, 148 ng/mL, 149 ng/mL, 150 ng/mL, 151 ng/mL, 152 ng/mL, 153 ng/mL, 154 ng/mL, 155 ng/mL, 156 ng/mL, 157 ng/mL, 158 ng/mL, 159 ng/mL, 160 ng/mL, 165 ng/mL, 170 ng/mL, 175 ng/mL, 180 ng/mL, 185 ng/mL, 190 ng/mL, 195 ng/mL, 200 ng/mL, 205 ng/mL, 210 ng/mL, 215 ng/mL, 220 ng/mL, 225 ng/mL, 230 ng/mL, 235 ng/mL, 240 ng/mL, 245 ng/mL, or 250 ng/mL or more bFGF, including values falling in between these concentrations.

Any of the cell culture media disclosed herein can also include angiopoietin 1 (ANG1). ANG1 is a member of the angiopoietin family of vascular growth factors that play a role in embryonic and postnatal angiogenesis. The cell culture media compositions for use in the methods of the present invention can include about 25-225 ng/mL of ANG1 such as about 50-200 ng/mL, about 100-200 ng/mL, about 100-150 ng/mL, or about 115-135 ng/mL, or such as any of about 75 ng/mL, 80 ng/mL, 85 ng/mL, 90 ng/mL, 95 ng/mL, 100 ng/mL, 105 ng/mL, 110 ng/mL, 115 ng/mL, 116 ng/mL, 117 ng/mL, 118 ng/mL, 119 ng/mL, 120 ng/mL, 121 ng/mL, 122 ng/mL, 123 ng/mL, 124 ng/mL, 125 ng/mL, 126 ng/mL, 127 ng/mL, 128 ng/mL, 129 ng/mL, 130 ng/mL, 131 ng/mL, 132 ng/mL, 133 ng/mL, 134 ng/mL, 135 ng/mL, 140 ng/mL, 141 ng/mL, 142 ng/mL, 143 ng/mL, 144 ng/mL, 145 ng/mL, 146 ng/mL, 147 ng/mL, 148 ng/mL, 149 ng/mL, 150 ng/mL, 151 ng/mL, 152 ng/mL, 153 ng/mL, 154 ng/mL, 155 ng/mL, 156 ng/mL, 157 ng/mL, 158 ng/mL, 159 ng/mL, 160 ng/mL, 165 ng/mL, 170 ng/mL, 175 ng/mL, 180 ng/mL, 185 ng/mL, 190 ng/mL, 195 ng/mL, 200 ng/mL, 205 ng/mL, 210 ng/mL, 215 ng/mL, 220 ng/mL, 225 ng/mL, 230 ng/mL, 235 ng/mL, 240 ng/mL, 245 ng/mL, or 250 ng/mL or more ANG1, including values falling in between these concentrations.

Interleukin 10 (IL-10) can also be a component of any of the cell culture media compositions disclosed herein. IL-10 is a cytokine with multiple, pleiotropic, effects in immunoregulation and inflammation. It downregulates the expression of Thl cytokines, MHC class II antigens, and co-stimulatory molecules on macrophages. It also enhances B cell survival, proliferation, and antibody production. IL-10 can block NF-κB activity, and is involved in the regulation of the JAK-STAT signaling pathway. The cell culture media compositions for use in the methods of the present invention can include about 1-25 ng/mL of IL-10 such as about 5-20 ng/mL, 10-20 ng/mL, or 12-18 ng/mL, such as any of about 1 ng/mL, 2 ng/mL, 3 ng/mL, 4 ng/mL, 5 ng/mL, 6 ng/mL, 7 ng/mL, 8 ng/mL, 9 ng/mL, 10 ng/mL, 11 ng/mL, 12 ng/mL, 13 ng/mL, 14 ng/mL, 15 ng/mL, 16 ng/mL, 17 ng/mL, 18 ng/mL, 19 ng/mL, 20 ng/mL, 21 ng/mL, 22 ng/mL, 23 ng/mL, 24 ng/mL, or 25 ng/mL of IL-10.

Interleukin 3 (IL-3) can also be a component of any of the cell culture media compositions disclosed herein. IL-3 is a cytokine with multiple, pleiotropic, effects in immunoregulation and inflammation. The cell culture media compositions for use in the methods of the present invention can include about 1-25 ng/mL of IL-3 such as about 5-20 ng/mL, 10-20 ng/mL, or 12-18 ng/mL, such as any of about 1 ng/mL, 2 ng/mL, 3 ng/mL, 4 ng/mL, 5 ng/mL, 6 ng/mL, 7 ng/mL, 8 ng/mL, 9 ng/mL, 10 ng/mL, 11 ng/mL, 12 ng/mL, 13 ng/mL, 14 ng/mL, 15 ng/mL, 16 ng/mL, 17 ng/mL, 18 ng/mL, 19 ng/mL, 20 ng/mL, 21 ng/mL, 22 ng/mL, 23 ng/mL, 24 ng/mL, or 25 ng/mL of IL-3. In some embodiments, the cell culture media compositions for use in the methods of the present invention can include concentrations at 25 ng/mL or above. Accordingly, concentrations of IL-3 also include 10-140 ng/mL, about 30-130, ng/mL about 50-120 ng/mL, about 70-110 ng/mL, or about 95-105 ng/mL, or such as any of about 30 ng/mL, 35 ng/mL, 40 ng/mL, 41 ng/mL, 42 ng/mL, 43 ng/mL, 44 ng/mL, 45 ng/mL, 46 ng/mL, 47 ng/mL, 48 ng/mL, 49 ng/mL, 50 ng/mL, 51 ng/mL, 52 ng/mL, 53 ng/mL, 54 ng/mL, 55 ng/mL, 56 ng/mL, 57 ng/mL, 58 ng/mL, 59 ng/mL, 60 ng/mL, 65 ng/mL, 70 ng/mL, 75 ng/mL, 80 ng/mL, 85 ng/mL, 90 ng/mL, 95 ng/mL, 100 ng/mL, 110 ng/mL, 115 ng/mL, 120 ng/mL, 125 ng/mL, 130 ng/mL, 135 ng/mL, 140 ng/mL, 145 ng/mL, 150 ng/mL, 155 ng/mL 160 ng/mL, 165 ng/mL, 170 ng/mL, 175 ng/mL, 180 ng/mL 185 ng/mL, 190 ng/mL, 200 ng/mL, or more IL-3, including values falling in between these concentrations. In some embodiments, the concentration of IL-3 in the media is about 100 ng/mL.

Interleukin 6 (IL-6) can also be a component of any of the cell culture media compositions disclosed herein. IL-6 is a cytokine with multiple, pleiotropic, effects in immunoregulation and inflammation. The cell culture media compositions for use in the methods of the present invention can include about 1-25 ng/mL of IL-6 such as about 5-20 ng/mL, 10-20 ng/mL, or 12-18 ng/mL, such as any of about 1 ng/mL, 2 ng/mL, 3 ng/mL, 4 ng/mL, 5 ng/mL, 6 ng/mL, 7 ng/mL, 8 ng/mL, 9 ng/mL, 10 ng/mL, 11 ng/mL, 12 ng/mL, 13 ng/mL, 14 ng/mL, 15 ng/mL, 16 ng/mL, 17 ng/mL, 18 ng/mL, 19 ng/mL, 20 ng/mL, 21 ng/mL, 22 ng/mL, 23 ng/mL, 24 ng/mL, or 25 ng/mL of IL-6. In some embodiments, the cell culture media compositions for use in the methods of the present invention can include concentrations at 25 ng/mL or above. Accordingly, concentrations of IL-6 also include 10-140 ng/mL, about 30-130, ng/mL about 50-120 ng/mL, about 70-110 ng/mL, or about 95-105 ng/mL, or such as any of about 30 ng/mL, 35 ng/mL, 40 ng/mL, 41 ng/mL, 42 ng/mL, 43 ng/mL, 44 ng/mL, 45 ng/mL, 46 ng/mL, 47 ng/mL, 48 ng/mL, 49 ng/mL, 50 ng/mL, 51 ng/mL, 52 ng/mL, 53 ng/mL, 54 ng/mL, 55 ng/mL, 56 ng/mL, 57 ng/mL, 58 ng/mL, 59 ng/mL, 60 ng/mL, 65 ng/mL, 70 ng/mL, 75 ng/mL, 80 ng/mL, 85 ng/mL, 90 ng/mL, 95 ng/mL, 100 ng/mL, 110 ng/mL, 115 ng/mL, 120 ng/mL, 125 ng/mL, 130 ng/mL, 135 ng/mL, 140 ng/mL, 145 ng/mL, 150 ng/mL, 155 ng/mL 160 ng/mL, 165 ng/mL, 170 ng/mL, 175 ng/mL, 180 ng/mL 185 ng/mL, 190 ng/mL, 200 ng/mL, or more IL-6, including values falling in between these concentrations. In some embodiments, the concentration of IL-6 in the media is about 100 ng/mL.

The cell culture media disclosed herein can also contain vascular endothelial growth factor 165 (VEGF165), which belongs to the PDGF/VEGF growth factor family. Many cell types secrete VEGF165, which it is a potent angiogenic factor and mitogen that stimulates proliferation, migration, and formation of endothelial cells. The cell culture media compositions for use in the methods of the present invention can include about 5-100 ng/mL of VEGF165, such as about 10-90 ng/mL, about 20-80, ng/mL about 30-70 ng/mL, about 40-60 ng/mL, or about 45-55 ng/mL, or such as any of about 5 ng/mL, 10 ng/mL, 15 ng/mL, 20 ng/mL, 25 ng/mL, 30 ng/mL, 35 ng/mL, 40 ng/mL, 41 ng/mL, 42 ng/mL, 43 ng/mL, 44 ng/mL, 45 ng/mL, 46 ng/mL, 47 ng/mL, 48 ng/mL, 49 ng/mL, 50 ng/mL, 51 ng/mL, 52 ng/mL, 53 ng/mL, 54 ng/mL, 55 ng/mL, 56 ng/mL, 57 ng/mL, 58 ng/mL, 59 ng/mL, 60 ng/mL, 65 ng/mL, 70 ng/mL, 75 ng/mL, 80 ng/mL, 85 ng/mL, 90 ng/mL, 95 ng/mL, 100 ng/mL or more VEGF165, including values falling in between these concentrations.

The cell culture media disclosed herein can also contain vascular endothelial growth factor C (VEGF-C), which belongs to the PDGF/VEGF growth factor family. Many cell types secrete VEGF-C, which functions in angiogenesis, and endothelial cell growth, stimulating proliferation and migration and also has effects on the permeability of blood vessels. The cell culture media compositions for use in the methods of the present invention can include about 50-1000 ng/mL of VEGF-C, such as about 100-900 ng/mL, about 200-800, ng/mL about 300-700 ng/mL, about 400-600 ng/mL, or about 450-550 ng/mL, or such as any of about 50 ng/mL, 100 ng/mL, 150 ng/mL, 200 ng/mL, 250 ng/mL, 300 ng/mL, 350 ng/mL, 400 ng/mL, 410 ng/mL, 420 ng/mL, 430 ng/mL, 440 ng/mL, 450 ng/mL, 460 ng/mL, 470 ng/mL, 480 ng/mL, 490 ng/mL, 500 ng/mL, 510 ng/mL, 520 ng/mL, 530 ng/mL, 540 ng/mL, 550 ng/mL, 560 ng/mL, 570 ng/mL, 580 ng/mL, 590 ng/mL, 600 ng/mL, 650 ng/mL, 700 ng/mL, 750 ng/mL, 800 ng/mL, 850 ng/mL, 900 ng/mL, 950 ng/mL, 1000 ng/mL or more VEGF-C, including values falling in between these concentrations.

In yet additional embodiments, the cell culture media compositions disclosed herein can contain laminins, which are high-molecular weight (˜400 kDa) proteins of the extracellular matrix. They are a major component of the basal lamina (one of the layers of the basement membrane), a protein network foundation for most cells and organs. The laminins are an important and biologically active part of the basal lamina, influencing cell differentiation, migration, and adhesion. The cell culture media compositions for use in the methods of the present invention can include about 500-1000 ng/mL laminin, such as about 600-900 ng/mL, about 700-800 ng/mL, about 725-775 ng/mL, or about 745-755 ng/mL, or such as any of about 500 ng/mL, 525 ng/mL, 550 ng/mL, 575 ng/mL, 600 ng/mL, 625 ng/mL, 650 ng/mL, 675 ng/mL, 700 ng/mL, 705 ng/mL, 710 ng/mL, 715 ng/mL, 720 ng/mL, 725 ng/mL, 730 ng/mL, 735 ng/mL, 740 ng/mL, 741 ng/mL, 742 ng/mL, 743 ng/mL, 744 ng/mL, 745 ng/mL, 746 ng/mL, 747 ng/mL, 748 ng/mL, 749 ng/mL, 750 ng/mL, 751 ng/mL, 752 ng/mL, 753 ng/mL, 754 ng/mL, 755 ng/mL, 756 ng/mL, 757 ng/mL, 758 ng/mL, 759 ng/mL, 760 ng/mL, 765 ng/mL, 770 ng/mL, 775 ng/mL, 780 ng/mL, 785 ng/mL, 790 ng/mL, 795 ng/mL, 800 ng/mL, 825 ng/mL, 850 ng/mL, 875 ng/mL, 900 ng/mL, 925 ng/mL, 950 ng/mL, 975 ng/mL, 1000 ng/mL or more laminin, including values falling in between these concentrations.

C. Other Small Molecules

The cell culture media for use in the methods disclosed herein can additionally contain various small molecule inhibitors, such as a caspase inhibitors, DNA methylation inhibitors, P38 MAPK inhibitors, glycogen synthase kinase 3 (GSK3) inhibitors, and/or JAK/STAT inhibitors. In one embodiment, the DMSO concentration of the cell culture media does not exceed 0.025% v/v.

In some embodiments, the cell culture media for use in the methods disclosed herein includes one or more a caspase inhibitors. Caspases are a family of cysteine proteases that play essential roles in apoptosis (programmed cell death), necrosis, and inflammation. As of November 2009, twelve caspases have been identified in humans. There are two types of apoptotic caspases: initiator (apical) caspases and effector (executioner) caspases. Initiator caspases (e.g., CASP2, CASP8, CASP9, and CASP10) cleave inactive pro-forms of effector caspases, thereby activating them. Effector caspases (e.g., CASP3, CASP6, CASP7) in turn cleave other protein substrates within the cell, to trigger the apoptotic process. The cell culture media compositions for use in the methods of the present invention can include about 1-10 μg/mL caspase inhibitor, such as any of about 2-8 μg/mL, about 3-7 μg/mL, or about 4-6 μg/mL, or such as any of about 1 μg/mL, 2 μg/mL, 3 μg/mL, 4 μg/mL, 5 μg/mL, 6 μg/mL, 7 μg/mL, 8 μg/mL, 9 μg/mL, 10 μg/mL or more caspase inhibitor. In one embodiment, the caspase inhibitor is Z-VAD-FMK.

The cell culture media for use in the methods disclosed herein can include one or more DNA methylation inhibitors. DNA methylation is a process by which methyl groups are added to DNA which modifies its function. When located in a gene promoter, DNA methylation typically acts to repress gene transcription. The cell culture media compositions for use in the methods of the present invention can include about 300-700 nM DNA methylation inhibitors, such as about 350-650 nM, about 400-600 nM, about 450-550 nM, about 475-525 nM, or about 490-510 nM or such as any of about 300 nM, 325 nM, 350 nM, 400 nM, 425 nM, 430 nM, 435 nM, 440 nM, 445 nM, 450 nM, 455 nM, 460 nM, 465 nM, 470 nM, 475 nM, 480 nM, 485 nM, 490 nM, 491 nM, 492 nM, 493 nM, 494 nM, 495 nM, 496 nM, 497 nM, 498 nM, 499 nM, 500 nM, 501 nM, 502 nM, 503 nM, 504 nM, 505 nM, 506 nM, 507 nM, 508 nM, 509 nM, 510 nM, 515 nM, 520 nM, 525 nM, 530 nM, 535 nM, 540 nM, 545 nM, 550 nM, 555 nM, 560 nM, 565 nM, 570 nM, 575 nM, 600 nM, 625 nM, 650 nM, 675 nM, 700 nM, or more DNA methylation inhibitors, including values falling in between these concentrations. In some embodiments, the DNA methylation inhibitor is epigallocatechin gallate (EGCG). In other embodiments, the cell culture media compositions for use in the methods of the present invention can include about 0.25-3 μM DNA methylation inhibitors, such as about 0.5-2.5 μM, about 1-2 μM, or about 1.25-1.75 μM, such as any of about 0.5 μM, 1 μM, 1.5 μM, 2 μM, 2.5 μM, or 3 μM or more DNA methylation inhibitors, including values falling in between these concentrations. In some embodiments, the DNA methylation inhibitor is Oct4-activating compound 1 (OAC1).

Any of the cell culture media disclosed herein can also include a P38 MAPK inhibitor. P38 mitogen-activated protein kinases are a class of mitogen-activated protein kinases that are responsive to stress stimuli, such as cytokines, ultraviolet irradiation, heat shock, and osmotic shock, and are involved in cell differentiation, apoptosis and autophagy. The cell culture media compositions for use in the methods of the present invention can include about 400-800 nM P38 MAPK inhibitor, such as about 500-700 nM, about 550-650 nM, about 600-650 nM, or about 615-635 nM, or such as any of about 400 nM, 425 nM, 450 nM, 475 nM, 500 nM, 525 nM, 550 nM, 575 nM, 600 nM, 605 nM, 610 nM, 615 nM, 616 nM, 617 nM, 618 nM, 619 nM, 620 nM, 621 nM, 622 nM, 623 nM, 624 nM, 625 nM, 626 nM, 627 nM, 628 nM, 629 nM, 630 nM, 631 nM, 632 nM, 633 nM, 634 nM, 635 nM, 640 nM, 645 nM, 650 nM, 655 nM, 660 nM, 665 nM, 670 nM, 675 nM, 680 nM, 685 nM, 690 nM, 695 nM, 700 nM, 725 nM, 750 nM, 775 nM, 800 nM, or more P38 MAPK inhibitor, including values falling in between these concentrations. In some embodiments, the P38 MAPK inhibitor is BIRB796.

In yet additional embodiments, the cell culture media compositions disclosed herein can contain a glycogen synthase kinase 3 (GSK3) inhibitor. GSK3 is a serine/threonine protein kinase that mediates the addition of phosphate molecules onto serine and threonine amino acid residues. Phosphorylation of a protein by GSK-3 usually inhibits the activity of its downstream target. GSK-3 is active in a number of central intracellular signaling pathways, including cellular proliferation, migration, glucose regulation, and apoptosis. The cell culture media compositions for use in the methods of the present invention can include about 0.25-2 μM GSK3 inhibitor, such as about 0.5-1.5 μM, or 1.75-1.25 μM, such as about 0.25 μM, 0.3 μM, 0.4 μM, 0.5 μM, 0.6 μM, 0.7 μM, 0.8 μM, 0.9 μM, 1 μM, 1.1 μM, 1.2 μM, 1.3 μM, 1.4 μM, 1.5 μM, 1.6 μM, 1.7 μM, 1.8 μM, 1.9 μM, or 2 μM or more GSK3 inhibitor, including values falling in between these concentrations. In some embodiments, the GSK3 inhibitor is CHIR99021.

In further embodiments, the cell culture media compositions disclosed herein can additionally contain a retinoic acid receptor (RAR) antagonist or the media can include a controlled or reduced amount of retinoic acid to restrict retinoic acid signaling. The RAR is a nuclear receptor as well as a transcription factor that is activated by both all-trans retinoic acid and 9-cis retinoic acid. In some embodiments retinoic acid signaling is reduced by limiting the amount of retinoic acid in the media.

In some embodiments, the cell culture media compositions disclosed herein can additionally contain a retinoic acid receptor (RAR) antagonist. The cell culture media compositions for use in the methods of the present invention can include about 10-300 nM RAR antagonist, such as about 25-175 nM, about 50-150, or about 75-125, or such as any of about 10 nM, 15 nM, 20 nM, 25 nM, 30 nM, 35 nM, 40 nM, 45 nM, 50 nM, 55 nM, 60 nM, 65 nM, 70 nM 75 nM, 80 nM, 85 nM, 90 nM, 95 nM, 100 nM, 105 nM, 110 nM, 115 nM, 120 nM, 125 nM, 130 nM, 135 nM, 140 nM, 145 nM, 150 nM, 155 nM, 160 nM, 165 nM, 170 nM, 175 nM, 180 nM, 185 nM, 190 nM, 191 nM, 192 nM, 193 nM, 194 nM, 195 nM, 196 nM, 197 nM, 198 nM, 199 nM, 200 nM, 201 nM, 202 nM, 203 nM, 204 nM, 205 nM, 206 nM, 207 nM, 208 nM, 209 nM, 210 nM, 215 nM, 220 nM, 225 nM, 230 nM, 235 nM, 240 nM, 241 nM, 242 nM, 243 nM, 244 nM, 245 nM, 246 nM, 247 nM, 248 nM, 249 nM, 250 nM, 251 nM, 252 nM, 253 nM, 254 nM, 255 nM, 256 nM, 257 nM, 258 nM, 259 nM, 260 nM, 265 nM, 270 nM, 275 nM, 280 nM, 285 nM, 290 nM, 295 nM, 300 nM or more RAR antagonist, including values falling in between these concentrations. In some embodiments, the RAR antagonist is ER50891. In some embodiments, the concentration of ER50891 is about 100 nM.

The cell culture media disclosed herein can also include a JAK/STAT inhibitor. The JAK-STAT signaling pathway transmits information from extracellular chemical signals to the nucleus resulting in DNA transcription and expression of genes involved in immunity, proliferation, differentiation, apoptosis and oncogenesis. The cell culture media compositions for use in the methods of the present invention can include about 300-700 nM JAK/STAT inhibitor, such as about 350-650 nM, about 400-600 nM, about 450-550 nM, about 475-525 nM, or about 490-510 nM or such as any of about 300 nM, 325 nM, 350 nM, 400 nM, 425 nM, 430 nM, 435 nM, 440 nM, 445 nM, 450 nM, 455 nM, 460 nM, 465 nM, 470 nM, 475 nM, 480 nM, 485 nM, 490 nM, 491 nM, 492 nM, 493 nM, 494 nM, 495 nM, 496 nM, 497 nM, 498 nM, 499 nM, 500 nM, 501 nM, 502 nM, 503 nM, 504 nM, 505 nM, 506 nM, 507 nM, 508 nM, 509 nM, 510 nM, 515 nM, 520 nM, 525 nM, 530 nM, 535 nM, 540 nM, 545 nM, 550 nM, 555 nM, 560 nM, 565 nM, 570 nM, 575 nM, 600 nM, 625 nM, 650 nM, 675 nM, 700 nM, or more JAK/STAT inhibitor, including values falling in between these concentrations. In some embodiments, the JAK/STAT inhibitor is Tofacitinib.

In addition to the inhibitor molecules described above, any of the cell culture media compositions disclosed herein can also contain fetal bovine serum (FBS) in concentrations ranging from 1-20% v/v, such as about 2-18% v/v, about 5-15% v/v, about 7.5-12.5% v/v or such as any of about 1% v/v, 2% v/v, 3% v/v, 4% v/v, 5% v/v, 6% v/v, 7% v/v, 8% v/v, 9% v/v, 10% v/v, 11% v/v, 12% v/v, 13% v/v, 14% v/v, 15% v/v, 16% v/v, 17% v/v, 18% v/v, 19% v/v, or 20% v/v or more FBS, including values falling in between these percentages. In some embodiments, the FBS is heat inactivated FBS. In some embodiments, the concentration of FBS in the medium is about 10% v/v.

In addition to the inhibitor molecules described above, any of the cell culture media compositions disclosed herein can also contain added salts, for example KCl, NaCl, MgCl, or CaCl₂. In one example, CaCl₂ may be added to achieve in concentrations ranging from 300-380 mOsm, such as about 300 mOsm, about 310 mOsm, about 320 mOsm, about 330 mOsm, about 340 mOsm, about 350 mOsm, about 360 mOsm, about 370 mOsm, about 380 mOsm, or more CaCl₂, including values falling in between these numbers. High osmolarity CaCl₂ may also be used to select against non-multipotent cells, selecting for an HSC phenotype.

In addition to the inhibitor molecules described above, any of the cell culture media compositions disclosed herein may be adjusted to comprise an overall higher osmolarity. Multipotent stem cells may be better adapted to withstand atypical osmolarity (e.g., a high osmolarity media may select against non-stem cell phenotypes.) Osmolarity may be adjusted, for example, by the addition of salts as above, or by glucose.

### IV. Methods of the Invention

A. Maintaining and/or Enhancing the Expansion of Hematopoietic Stem Cells in Culture

Provided herein are methods for maintaining and/or enhancing the expansion of hematopoietic stem cells (HSCs) in culture. The method involves contacting a source of CD34+ cells in culture with a PTEN inhibitor. In some embodiments, the PTEN inhibitor is SF1670 or a chemically altered version thereof. In some embodiments, the PTEN inhibitor is a chemically altered version of SF1670 described above. In some embodiments, the methods provided herein do not include a tetraspanin. In some embodiments, the methods provided herein also include a retinoic acid receptor (RAR) inhibitor or modulator. In some embodiments, the RAR inhibitor is ER50891.

1. Sources of CD34+ Cells

The methods of the present invention require a source of CD34+ blood cells, or in some examples CD34low/−, CD133+ cells. These cells can be obtained from tissue sources such as, e.g., bone marrow, cord blood, placental blood, mobilized peripheral blood, non-mobilized peripheral blood, or the like, or combinations thereof.

CD34+ cells can, in certain embodiments, express or lack the cellular marker CD133. Thus, in specific embodiments, the hematopoietic cells useful in the methods disclosed herein are CD34+CD133+ or CD34+CD133-. In other embodiments, CD34+ cells can express or lack the cellular marker CD90. As such, in these embodiments, the hematopoietic cells useful in the methods disclosed herein are CD34+CD90+ or CD34+CD90-. Thus, populations of CD34+ cells, or in some examples CD34low/−, CD133+ cells, can be selected for use in the methods disclosed herein on the basis of the presence of markers that indicate an undifferentiated state, or on the basis of the absence of lineage markers indicating that at least some lineage differentiation has taken place.

CD34+ cells used in the methods provided herein can be obtained from a single individual, e.g., from a source of non-mobilized peripheral blood, or from a plurality of individuals, e.g., can be pooled. Where the CD34+ cells are obtained from a plurality of individuals and pooled, it is preferred that the hematopoietic cells be obtained from the same tissue source. Thus, in various embodiments, the pooled hematopoietic cells are all from, for example, placenta, umbilical cord blood, peripheral blood (mobilized or non-mobilized), and the like.

Interestingly, cells enhanced and expanded by methods of the present invention are, for example, phenotypically similar to cord blood. Accordingly, it may be possible to use cells expanded and enhanced by methods described herein as a source for further expansion and enhancement. For example, it may be possible, following an initial expansion and enhancement to allow, or gently encourage, cells toward differentiation. These cells may be allowed to expand and can then be brought back from a differentiated, or near differentiated state, by following the methods of the invention utilized in the initial expansion/enhancement step. This expansion of differentiated, or nearly differentiated cells which can then be returned to a multipotent state may occur over multiple cycles.

CD34+ cells, or in some examples CD34low/−, CD133+ cells, can be isolated from a source using any conventional means known in the art such as, without limitation, positive selection against stem cell markers, negative selection against lineage markers, size exclusion, detection of metabolic differences in the cells, detection of differences in clearance or accumulation of a substance by the cell, adhesion differences, direct culturing of buffy coat under conditions exclusively supportive for stem cells. The source of CD34+ cells for use in the methods of the present invention can contain a number of sub-species of hematopoietic progenitor cells including, without limitation, one or more of CD34+ hematopoietic progenitors; CD34+ early hematopoietic progenitors and/or stem cells; CD133+ early hematopoietic progenitors and/or stem cells; and/or CD90+ early hematopoietic progenitors and/or stem cells.

2. Maintaining HSCs in Culture

CD34+ cells derived from the sources described above are cultured in any of the cell culture media described herein. These media maintain and enhance the hematopoietic stem cell phenotype. Furthermore, the addition of a PTEN inhibitor augments these effects. Specifically, use of a PTEN inhibitor in the culture media increases the rate of expansion of HSCs while maintaining (and usually improving) all measured stem cell markers (such as, but not limited to CD133 and CD90). These improvements can be seen after as little as 3 days of culture. In some embodiments, the PTEN inhibitor is SF1670 or a chemically altered version thereof. In some embodiments, the media provided herein does not include a tetraspanin. In some embodiments, media provided herein also includes a retinoic acid receptor (RAR) inhibitor or modulator. In some embodiments, the RAR inhibitor is ER50891.

In particular, source cells cultured in any of the cell culture media described herein exhibit increased numbers of CD133+ and/or CD90+ positive cells compared to source cells that are not cultured in any of the media described herein after about any of 1, 2, 3, 4, 5, 6, 7, 8, 9, 10, 11, 12, 13, 14, 15, 20, 25, 30, 35, 40, 45, or 50 days or more in culture. Specifically, source cells cultured in the media described herein using the methods disclosed herein exhibited around 1.5, 1.6, 1.7, 1.8, 1.9, 2, 2.1, 2.2, 2.3, 2.4, 2.5, 2.6, 2.7, 2.8, 2.9, 3, 3.5, 4, 4.5, 5, 7.5, 10, 15, 20, 25, 30, 35, 40, 45, 50 or more times the number of CD133+ and/or CD90+ positive cells compared to source cells that are not cultured in any of the media described herein after about any of 3, 4, 5, 6, 7, 8, 9, 10, 11, 12, 13, 14, 15, 20, 25, 30, 35, 40, 45, or 50 days or more in culture.

Source cells cultured in the cell culture media described herein also exhibit increased number of CD90+/CD38 low/− cells compared to source cells that are not cultured in any of the media described herein after about any of 3, 4, 5, 6, 7, 8, 9, 10, 11, 12, 13, 14, 15, 20, 25, 30, 35, 40, 45, or 50 days or more in culture. Specifically, source cells cultured in the media described herein using the methods disclosed herein exhibited around 1.5, 1.6, 1.7, 1.8, 1.9, 2, 2.1, 2.2, 2.3, 2.4, 2.5, 2.6, 2.7, 2.8, 2.9, 3, 3.5, 4, 4.5, 5, 7.5, 10, 12.5, 15, 17.5, or 20 or more times the number of CD90+/CD38 low/− cells compared to source cells that are not cultured in any of the media described herein after about any of 3, 4, 5, 6, 7, 8, 9, 10, 11, 12, 13, 14, 15, 20, 25, 30, 35, 40, 45, or 50 days or more in culture.

The cell culture methods disclosed herein include culturing cells under low oxygen conditions. As used herein, the phrase “low oxygen conditions” refers to an atmosphere to which the cultured cells are exposed having less than about 10% oxygen, such as any of about 10%, 9.5, 9%, 8.5%, 8%, 7.5%, 7%, 6.5%, 6%, 5.5%, or 5%, 4.5%, 4%, 3.5%, 3%, 2.75%, 2.5%, 2.25%, 2%, 1.75%, 1.5%%, 1.25%, 1%, 0.9%, 0.8%, 0.7%, 0.6%, or 0.5% or less oxygen. “Low oxygen conditions” can also refer to any range in between 0.5% and 10% oxygen. Control of atmospheric oxygen in cell culture can be performed by any means known in the art, such as by addition of nitrogen.

The invention also contemplates populations of cells that are made by the methods described herein. Populations of cells containing HSCs provided herein confer the advantages found in cord blood. A person of skill in the art would readily recognize the characteristics of stem cells from cord blood and the advantageous properties therein. In some embodiments, at least 10%, 20%, 30%, 40%, 50%, 60%, 70%, 80%, 90%, or 100% of the populations of cells containing HSCs provided herein are expanded HSCs. In some embodiments, the expanded HSCs in the populations of cells have retained their stem cell phenotype for an extended period of time. For example, in some embodiments, populations of cells containing HSCs include expanded HSCs with cell surface phenotypes that include CD45+, CD34+, CD133+, CD90+, CD45RA-, and/or CD38 low/− and have been cultured in vitro for at least 3, 7, 10, 13, 14, 20, 25, 30, 40, or 50 or more days. In some embodiments, populations of cells containing HSCs include expanded HSCs with cell surface phenotypes that includes CD133+ and/or CD90+ and have been cultured in vitro for at least 3, 7, 10, 13, 14 or more days.

B. Methods of Treatment

Provided herein are methods for treating an individual in need of hematopoietic reconstitution. The method involves administering to the individual a therapeutic agent containing any of the cultured HSCs derived according to the methods of the present invention.

One of ordinary skill in the art may readily determine the appropriate concentration, or dose of the cultured HSCs disclosed herein for therapeutic administration. The ordinary artisan will recognize that a preferred dose is one that produces a therapeutic effect, such as preventing, treating and/or reducing diseases, disorders and injuries, in a patient in need thereof. Of course, proper doses of the cells will require empirical determination at time of use based on several variables including but not limited to the severity and type of disease, injury, disorder or condition being treated; patient age, weight, sex, health; other medications and treatments being administered to the patient; and the like.

An effective amount of cells may be administered in one dose, but is not restricted to one dose. Thus, the administration can be two, three, four, five, six, seven, eight, nine, ten, eleven, twelve, thirteen, fourteen, fifteen, sixteen, seventeen, eighteen, nineteen, twenty, or more, administrations of pharmaceutical composition. Where there is more than one administration of a therapeutic agent in the present methods, the administrations can be spaced by time intervals of one minute, two minutes, three, four, five, six, seven, eight, nine, ten, or more minutes, by intervals of about one hour, two hours, three, four, five, six, seven, eight, nine, ten, 11, 12, 13, 14, 15, 16, 17, 18, 19, 20, 21, 22, 23, 24 hours, and so on. In the context of hours, the term “about” means plus or minus any time interval within 30 minutes. The administrations can also be spaced by time intervals of one day, two days, three days, four days, five days, six days, seven days, eight days, nine days, ten days, 11 days, 12 days, 13 days, 14 days, 15 days, 16 days, 17 days, 18 days, 19 days, 20 days, 21 days, and combinations thereof. The invention is not limited to dosing intervals that are spaced equally in time, but encompass doses at non-equal intervals.

A dosing schedule of, for example, once/week, twice/week, three times/week, four times/week, five times/week, six times/week, seven times/week, once every two weeks, once every three weeks, once every four weeks, once every five weeks, and the like, is available for the invention. The dosing schedules encompass dosing for a total period of time of, for example, one week, two weeks, three weeks, four weeks, five weeks, six weeks, two months, three months, four months, five months, six months, seven months, eight months, nine months, ten months, eleven months, and twelve months.

Provided are cycles of the above dosing schedules. The cycle can be repeated about, e.g., every seven days; every 14 days; every 21 days; every 28 days; every 35 days; 42 days; every 49 days; every 56 days; every 63 days; every 70 days; and the like. An interval of non-dosing can occur between a cycle, where the interval can be about, e.g., seven days; 14 days; 21 days; 28 days; 35 days; 42 days; 49 days; 56 days; 63 days; 70 days; and the like. In this context, the term “about” means plus or minus one day, plus or minus two days, plus or minus three days, plus or minus four days, plus or minus five days, plus or minus six days, or plus or minus seven days.

Cells derived from the methods of the present invention may be formulated for administration according to any of the methods disclosed herein in any conventional manner using one or more physiologically acceptable carriers optionally comprising excipients and auxiliaries. Proper formulation is dependent upon the route of administration chosen. The compositions may also be administered to the individual in one or more physiologically acceptable carriers. Carriers for cells may include, but are not limited to, solutions of normal saline, phosphate buffered saline (PBS), lactated Ringer's solution containing a mixture of salts in physiologic concentrations, or cell culture medium.

The HSC populations of the invention and therapeutic agents comprising the same can be used to augment or replace bone marrow cells in bone marrow transplantation. Human autologous and allogenic bone marrow transplantation are currently used as therapies for diseases such as leukemia, lymphoma and other life-threatening disorders. The drawback of these procedures, however, is that a large amount of donor bone marrow must be removed to ensure that there are enough cells for engraftment.

The HSC populations of the invention and therapeutic agents comprising the same can provide stem cells and progenitor cells that would reduce the need for large bone marrow donation. It would also be possible, according to the methods of the invention, to obtain a small marrow donation and then expand the number of stem cells and progenitor cells culturing and expanding in the placenta before infusion or transplantation into a recipient. Alternatively, sufficient numbers of HSCs can be obtained according to the methods of the present invention using only non-mobilized peripheral blood, thereby completely eliminating the need for bone marrow donation altogether.

Compositions and methods of the present invention are useful in the expansion of stem cells. In some embodiments, the expansion can be rapid compared to traditional methods of expansion. In some embodiments, expansion may occur in the course of hours, days, or weeks (e.g., selective expansion can occur for about 2 hours, 4 hours, 6 hours, 8 hours, 12 hours, 16 hours, 20 hours, one day, two days, three days, four days, five days, six days, seven days, nine days, ten days, 11 days, 12 days, 13 days, two weeks, three weeks, four weeks, or more. In some embodiments, a stem cell population may be expanded in terms of total cell count by two-fold, three-fold, four-fold, five-fold, 6-fold, 7-fold, 8-fold, 9-fold, 10-fold, 15-fold, 20-fold, 30-fold, 40-fold, 50-fold, or more. In some embodiments, a stem cell population may be expanded in terms of the relative number of cells with a stem cell phenotype in a broader cell population (e.g. cells with a stem cell phenotype may make up about 1%, 2/%, 3%, 4%, 5%, 6%, 7%, 8%, 9%, 10%, 15%, 20%, 30%, 40%, 50%, 60%, 70%, 80%, 90%, 95%, 97.5%, 98%, 99%, or 100% of a cell population). Expansion may be measured by a number of metrics including by doubling time for example, by the amount of time it takes for a total cell number to double (e.g., from 500 cells to 1,000 cells), or the time it takes for a relative percentage of the population to double (e.g., from 10% stem cells to 20% stem cells).

In another embodiment, the HSC populations of the invention and therapeutic agents comprising the same can be used in a supplemental treatment in addition to chemotherapy. Most chemotherapy agents used to target and destroy cancer cells act by killing all proliferating cells, i.e., cells going through cell division. Since bone marrow is one of the most actively proliferating tissues in the body, hematopoietic stern cells are frequently damaged or destroyed by chemotherapy agents and in consequence, blood cell production is diminishes or ceases. Chemotherapy must be terminated at intervals to allow the patient's hematopoietic system to replenish the blood cell supply before resuming chemotherapy. It may take a month or more for the formerly quiescent stem cells to proliferate and increase the white blood cell count to acceptable levels so that chemotherapy may resume (when again, the bone marrow stem cells are destroyed).

While the blood cells regenerate between chemotherapy treatments, however, the cancer has time to grow and possibly become more resistant to the chemotherapy drugs due to natural selection. Therefore, the longer chemotherapy is given and the shorter the duration between treatments, the greater the odds of successfully killing the cancer. To shorten the time between chemotherapy treatments, the HSC populations of the invention and therapeutic agents comprising the same cultured according to the methods of the invention could be introduced into the individual. Such treatment would reduce the time the individual would exhibit a low blood cell count, and would therefore permit earlier resumption of the chemotherapy treatment.

C. Methods for Producing a Cell Culture Medium

Further provided herein are methods for producing a cell culture medium (such as any of the cell culture media disclosed herein) for culturing hematopoietic stem cells (HSCs). The method involves combining a base or a feed medium; and a PTEN inhibitor. In some embodiments, the PTEN inhibitor is SF1670 or a chemically altered version thereof. In some embodiments, the PTEN inhibitor is a chemically altered version of SF1670 described above. In some embodiments, the methods provided herein also include a retinoic acid receptor (RAR) inhibitor or modulator. In some embodiments, the RAR inhibitor is ER50891. In additional embodiments, the method also includes combining one, two, or all three of stem cell factor (SCF), thrombopoietin (TPO), and/or fms-related tyrosine kinase 3 ligand (Flt31). The method can also include combining one or more of a caspase inhibitor, a DNA methylation inhibitor, a p38 MAPK inhibitor, a GSK3 inhibitor, an RAR receptor antagonist, an inhibitor of the JAK/STAT pathway, and/or FBS (such as, heat inactivated FBS). In some embodiments, the methods provided herein do not include a tetraspanin.

A “base medium,” as used herein, is a medium used for culturing cells which is, itself, directly used to culture the cells and is not used as an additive to other media, although various components may be added to a base medium. Examples of base media include, without limitation, DMEM medium, IMDM medium, StemSpan Serum-Free Expansion Medium (SFEM), 199/109 medium, HamF10/F12 medium, McCoy's 5A medium, Alpha MEM medium (without and with phenol red), and RPMI 1640 medium. A base medium may be modified, for example by the addition of salts, glucose, or other additives.

A “feed medium” is a medium used as a feed in a culture of a source of CD34+ cells (e.g. bone marrow, cord blood, mobilized peripheral blood, and non-mobilized peripheral blood cells). A feed medium, like a base medium, is designed with regard to the needs of the particular cells being cultured. Thus, a base medium can be used as a basis for designing a feed medium. A feed medium can have higher concentrations of most, but not all, components of a base culture medium. For example, some components, such as salts, may be kept at about 1× of the base medium concentration, as one would want to keep the feed isotonic with the base medium. Thus, in some embodiments, various components are added to keep the feed medium physiologic and others are added because they replenish nutrients to the cell culture. Other components, for example, nutrients, may be at about 2×, 3×, 4×, 5×, 6×, 7×, 8×, 9×, 10×, 12×, 14×, 16×, 20×, 30×, 50×, 100× or more of their normal concentrations in a base medium.

### V. Systems and Kits

Also provided herein are systems for maintaining and/or enhancing the expansion of hematopoietic stem cells in culture. This system includes a source of CD34+ cells in culture (such as a CD34+ cells from one or more of bone marrow, cord blood, mobilized peripheral blood, and non-mobilized peripheral blood) and any of the cell culture media compositions described herein. In a particular embodiment, the system of the present invention maintains low oxygen culturing conditions. As such, the system provides an atmosphere to which the cultured cells are exposed having less than about 10% oxygen, such as any of about 10%, 9.5, 9%, 8.5%, 8%, 7.5%, 7%, 6.5%, 6%, 5.5%, or 5%, 4.5%, 4%, 3.5%, 3%, 2.75%, 2.5%, 2.25%, 2%, 1.75%, 1.5%%, 1.25%, 1%, 0.9%, 0.8%, 0.7%, 0.6%, or 0.5% or less oxygen. In some embodiments, the system provides an atmosphere to which the culture cells are exposed having any range in between 0.5% and 10% oxygen. Control of atmospheric oxygen in the system can be accomplished by any means known in the art, such as by addition of nitrogen.

In additional aspects, the invention disclosed herein provides one or more kits. These kits can include either a base medium or a feed medium (such as, but not limited to, DMEM medium, IMDM medium, StemSpan Serum-Free Expansion Medium (SFEM), 199/109 medium, HamF10/F12 medium, McCoy's 5A medium, Alpha MEM medium (without and with phenol red), and RPMI 1640 medium) as well as a PTEN inhibitor. In some embodiments, the PTEN inhibitor is SF1670 or a chemically altered version thereof. In some embodiments, the kits provided herein do not include a tetraspanin.

The kit can also include written instructions for maintaining and/or enhancing the expansion of HSCs in culture by culturing the cells using the kit's cell culture media components. The kit can also include additional components for inclusion into the cell culture media, such as one or more of thrombopoietin (TPO), stem cell factor (SCF), insulin-like growth factor 1 (IGF-1), erythroid differentiation factor (EDF), hepatocyte growth factor (HGF), epidermal growth factor (EGF), heat shock factor (HSF), pleiotrophin (PTN), basic fibroblast growth factor (bFGF), angiopoietin 1 (ANG1), VEGF165, IL-10, laminin, caspase inhibitor(s), epigallocatechin gallate (EGCG), Oct4-activating compound 1 (OAC1), P38 MAPK inhibitor JAK/STAT inhibitors, IL-3, IL-6, human growth hormone (HGH), fms-related tyrosine kinase 3 ligand (FLT3L), VEGF-C and ALK5/SMAD modulators or inhibitors, and fetal bovine serum (FBS) (including heat-inactivated FBS).

In some embodiments, the kit also includes a retinoic acid receptor (RAR) inhibitor or modulator. In some embodiments, the RAR inhibitor or modulator is ER50891. In some embodiments, the kit includes also thrombopoietin (TPO), stem cell factor (SCF), insulin-like growth factor 1 (IGF-1), human growth hormone (HGH), fms-related tyrosine kinase 3 ligand (FLT3L), and fetal bovine serum (FBS).

It is intended that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

The invention can be further understood by reference to the following examples, which are provided by way of illustration and are not meant to be limiting.

### VI. Particular Embodiments of the Present Disclosure

### Embodiment 1

A method for expanding hematopoietic stem cells in culture, the method comprising contacting a source of CD34+ cells in culture with an effective amount of a phosphatase and tensin homolog (PTEN) inhibitor, thereby expanding hematopoietic stem cells in the culture.

### Embodiment 2

The method of embodiment 1, wherein the source of CD34+ cells is selected from the group consisting of bone marrow, cord blood, mobilized peripheral blood, and non-mobilized peripheral blood.

### Embodiment 3

The method of embodiment 1, wherein the source of CD34+ cells is non-mobilized peripheral blood.

### Embodiment 4

The method of embodiment 2 or embodiment 3, wherein the source of CD34+ cells comprises one or more of (a) CD34+ hematopoietic progenitors; (b) CD34+ early hematopoietic progenitors and/or stem cells; (c) CD133+ early hematopoietic progenitors and/or stem cells; and/or (d) CD90+ early hematopoietic progenitors and/or stem cells.

### Embodiment 5

The method of any one of embodiments 1-4, wherein the PTEN inhibitor is SF1670 or a chemically altered version thereof.

### Embodiment 6

The method of embodiments 5, wherein the PTEN inhibitor is a chemically altered version of SF1670.

### Embodiment 7

The method of embodiments 6, wherein the chemically altered version of SF1670 is a compound of any one of embodiments 75-111.

### Embodiment 8

The method of any one of embodiments 1-7, further comprising a retinoic acid receptor (RAR) inhibitor or modulator.

### Embodiment 9

The method of embodiment 8, wherein the retinoic acid receptor (RAR) inhibitor or modulator is ER50891.

### Embodiment 10

The method of any one of embodiments 1-9, wherein the method further comprises culturing the cells under low oxygen conditions.

### Embodiment 11

The method of embodiment 10, wherein low oxygen conditions comprise an atmosphere containing about 5% oxygen or less.

### Embodiment 12

The method of any one of embodiments 1-11, wherein the method further comprises contacting the cells with one or more agents selected from the group consisting of thrombopoietin (TPO), stem cell factor (SCF), hepatocyte growth factor (HGF), P38 MAPK inhibitor, epidermal growth factor (EGF), JAK/STAT inhibitors, IL-3, IL-6, human growth hormone (HGH), fms-related tyrosine kinase 3 ligand (FLT3L), VEGF-C and ALK5/SMAD modulators or inhibitors.

### Embodiment 13

The method of any one of embodiments 1-11, wherein the method further comprises contacting the cells with thrombopoietin (TPO), stem cell factor (SCF), and fms-related tyrosine kinase 3 ligand (FLT3L).

### Embodiment 14

The method of any one of embodiments 1-11, wherein the method further comprises contacting the cells with thrombopoietin (TPO) and stem cell factor (SCF).

### Embodiment 15

The method of any one of embodiments 1-14, wherein said method stabilizes the hematopoietic stem cell phenotype.

### Embodiment 16

The method of embodiment 15, wherein the hematopoietic stem cell phenotype comprises CD45+, CD34+, CD133+, CD90+, CD45RA−, CD38 low/−, and negative for major hematopoietic lineage markers including CD2, CD3, CD4, CD5, CD8, CD14, CD16, CD19, CD20, CD56.

### Embodiment 17

The method of any one of embodiments 1-16, wherein CD133+ and/or CD90+ positive cells are increased compared to cells in culture that are not contacted with a PTEN inhibitor or a chemically altered version thereof.

### Embodiment 18

The method of embodiment 17, wherein the cells exhibit at least about two times the number of CD133+ and/or CD90+ positive cells compared to cells in culture that are not contacted with a PTEN inhibitor or a chemically altered version thereof after 7 day in culture.

### Embodiment 19

The method of any one of embodiments 1-18, wherein the source of the CD34+ cells is a human being.

### Embodiment 20

A medium for expanding hematopoietic stem cells in culture comprising:
(a)-(i) a base medium or (ii) a feed medium; and
(b) a phosphatase and tensin homolog (PTEN) inhibitor.

### Embodiment 21

The medium of embodiment 20, wherein the PTEN inhibitor is SF1670 or a chemically altered version thereof.

### Embodiment 22

The method of embodiment 21, wherein the PTEN inhibitor is a chemically altered version of SF1670.

### Embodiment 23

The method of embodiment 22, wherein the chemically altered version of SF1670 is a compound of any one of claims 75-111.

### Embodiment 24

The medium of any one of claims 20-23, wherein the medium further comprises (c) a retinoic acid receptor (RAR) inhibitor or modulator.

### Embodiment 25

The medium of embodiment 24, wherein the retinoic acid receptor (RAR) inhibitor or modulator is ER50891.

### Embodiment 26

The medium of any one of embodiments 20-25, wherein the medium further comprises (c) one or more agents selected from the group consisting of thrombopoietin (TPO), stem cell factor (SCF), insulin-like growth factor 1 (IGF-1), erythroid differentiation factor (EDF), hepatocyte growth factor (HGF), epidermal growth factor (EGF), heat shock factor (HSF), pleiotrophin (PTN), basic fibroblast growth factor (bFGF), angiopoietin 1 (ANG1), VEGF165, IL-10, laminin, caspase inhibitor(s), epigallocatechin gallate (EGCG), Oct4-activating compound 1 (OAC1), P38 MAPK inhibitor JAK/STAT inhibitors, IL-3, IL-6, human growth hormone (HGH), fms-related tyrosine kinase 3 ligand (FLT3L), VEGF-C and ALK5/SMAD modulators or inhibitors, and fetal bovine serum (FBS).

### Embodiment 27

The medium of embodiment 26, wherein the FBS is heat inactivated.

### Embodiment 28

The medium of any one of embodiments 20-25, wherein the medium further comprises (c) thrombopoietin (TPO), stem cell factor (SCF), and fms-related tyrosine kinase 3 ligand (FLT3L).

### Embodiment 29

The medium of any one of embodiments 20-25, wherein the medium further comprises (c) thrombopoietin (TPO) and stem cell factor (SCF).

### Embodiment 30

The medium of any one of embodiments 20-29, wherein the base medium is a base salt medium.

### Embodiment 31

The medium of embodiment 27, wherein the base salt medium is alpha MEM.

### Embodiment 32

The medium of embodiment 30, wherein the base salt medium comprises a sufficient amount of CaCl₂ to adjust the base salt medium to 320-380 mOsm.

### Embodiment 33

A method for expanding hematopoietic stem cells in culture, the method comprising contacting a source of CD34+ cells in culture with the medium of any one of embodiments 20-32, thereby expanding hematopoietic stem cells in the culture.

### Embodiment 34

A system for expanding hematopoietic stem cells in culture, the system comprising (a) a source of CD34+ cells in culture; and (b) the medium of any one embodiments 20-32.

### Embodiment 35

The system of embodiment 34, wherein the source of CD34+ cells is selected from the group consisting of bone marrow, cord blood, mobilized peripheral blood, and non-mobilized peripheral blood.

### Embodiment 36

The system of embodiment 35, wherein the source of CD34+ cells is non-mobilized peripheral blood.

### Embodiment 37

The system of embodiment 35 or embodiment 36, wherein the source of CD34+ cells comprises one or more of (a) CD34+ hematopoietic progenitors; (b) CD34+ early hematopoietic progenitors and/or stem cells; (c) CD133+ early hematopoietic progenitors and/or stem cells; and/or (d) CD90+ early hematopoietic progenitors and/or stem cells.

### Embodiment 38

The system of any one of embodiments 34-37, further comprising (c) an atmosphere containing low oxygen.

### Embodiment 39

The system of embodiment 38, wherein the atmosphere contains about 5% oxygen or less.

### Embodiment 40

The system of any one of embodiments 34-39, wherein the source of CD34+ cells is a human being.

### Embodiment 41

A kit comprising: (i) a base medium or (ii) a feed medium; and (ii) a phosphatase and tensin homolog (PTEN) inhibitor.

### Embodiment 42

The kit of embodiment 41, wherein the PTEN inhibitor is SF1670 or a chemically altered version thereof.

### Embodiment 43

The kit of embodiment 42, wherein the PTEN inhibitor is a chemically altered version of SF1670.

### Embodiment 44

The kit of embodiment 43, wherein the chemically altered version of SF1670 is a compound of any one of embodiments 75-111.

### Embodiment 45

The kit of any one of embodiments 41-44, further comprising (c) written instructions for maintaining and/or expanding hematopoietic stem cells in culture.

### Embodiment 46

The kit of any one of embodiments 41-45, further comprising (d) a retinoic acid receptor (RAR) inhibitor or modulator.

### Embodiment 47

The kit of embodiment 46, wherein the retinoic acid receptor (RAR) inhibitor or modulator is ER50891.

### Embodiment 48

The kit of any one of embodiments 41-47, further comprising one or more agents selected from the group consisting of thrombopoietin (TPO), stem cell factor (SCF), insulin-like growth factor 1 (IGF-1), erythroid differentiation factor (EDF), hepatocyte growth factor (HGF), epidermal growth factor (EGF), heat shock factor (HSF), pleiotrophin (PTN), basic fibroblast growth factor (bFGF), angiopoietin 1 (ANG1), VEGF165, IL-10, laminin, caspase inhibitor(s), epigallocatechin gallate (EGCG), Oct4-activating compound 1 (OAC1), P38 MAPK inhibitor JAK/STAT inhibitors, IL-3, IL-6, human growth hormone (HGH), fms-related tyrosine kinase 3 ligand (FLT3L), VEGF-C and ALK5/SMAD modulators or inhibitors, and fetal bovine serum (FBS).

### Embodiment 49

The kit of embodiment 48, wherein the FBS is heat inactivated.

### Embodiment 50

The kit of any one of embodiments 41-47, further comprising (d) thrombopoietin (TPO), stem cell factor (SCF), and fms-related tyrosine kinase 3 ligand (FLT3L).

### Embodiment 51

The kit of any one of embodiments 41-47, further comprising (d) thrombopoietin (TPO) and stem cell factor (SCF).

### Embodiment 52

The kit of any one of embodiments 41-51, wherein the base medium is a base salt medium.

### Embodiment 53

The kit of embodiment 52, wherein the base salt medium is alpha MEM.

### Embodiment 54

The kit of embodiment 52, wherein the base salt medium comprises 320-380 mOsm CaCl₂.

### Embodiment 55

A population of hematopoietic stem cells produced by the method of any one of embodiments 1-19 or 33.

### Embodiment 56

A therapeutic agent comprising the population of hematopoietic stem cells of embodiment 55.

### Embodiment 57

A method of treating an individual in need of hematopoietic reconstitution, comprising administering to said individual the therapeutic agent of embodiment 56.

### Embodiment 58

The method of embodiment 57, wherein the individual is a bone marrow donor or recipient.

### Embodiment 59

The method of embodiment 58, wherein the individual is diagnosed with cancer.

### Embodiment 60

The method of embodiment 59, wherein the method is used as a supplemental treatment in addition to chemotherapy.

### Embodiment 61

The method of embodiment 60, wherein the method is used to shorten the time between chemotherapy treatments.

### Embodiment 62

The method of embodiment 57, wherein the individual is diagnosed with an autoimmune disease.

### Embodiment 63

A method for producing a cell culture media for culturing hematopoietic stem cells (HSC), the method comprising: combining (a) a base or a feed medium; and (b) a phosphatase and tensin homolog (PTEN) inhibitor.

### Embodiment 64

The method of embodiment 63, wherein the PTEN inhibitor is SF1670 or a chemically altered version thereof.

### Embodiment 65

The method of embodiment 64, wherein the PTEN inhibitor is a chemically altered version of SF1670.

### Embodiment 66

The method of embodiment 65, wherein the chemically altered version of SF1670 is a compound of any one of claims 75-111.

### Embodiment 67

The method of any one of embodiments 63-66, further comprising (c) a retinoic acid receptor (RAR) inhibitor or modulator.

### Embodiment 68

The medium of embodiment 67, wherein the retinoic acid receptor (RAR) inhibitor or modulator is ER50891.

### Embodiment 69

The method of any one of embodiments 63-68, further comprising thrombopoietin (TPO), stem cell factor (SCF), and/or fms-related tyrosine kinase 3 ligand (FLT3L).

### Embodiment 70

The method of any one of embodiments 63-68, further comprising thrombopoietin (TPO) and stem cell factor (SCF).

### Embodiment 71

The method of any one of embodiments 63-70, further comprising combining one or more of insulin-like growth factor 1 (IGF-1), erythroid differentiation factor (EDF), hepatocyte growth factor (HGF), epidermal growth factor (EGF), heat shock factor (HSF), pleiotrophin (PTN), basic fibroblast growth factor (bFGF), angiopoietin 1 (ANG1), VEGF165, IL-10, laminin, caspase inhibitor(s), epigallocatechin gallate (EGCG), Oct4-activating compound 1 (OAC1), P38 MAPK inhibitor JAK/STAT inhibitors, IL-3, IL-6, human growth hormone (HGH), fms-related tyrosine kinase 3 ligand (FLT3L), VEGF-C and ALK5/SMAD modulators or inhibitors, and fetal bovine serum (FBS).

### Embodiment 72

The method of embodiment 71, wherein the FBS is heat-inactivated FBS.

### Embodiment 73

The method of any one of embodiments 63-69, further comprising (d) insulin-like growth factor 1 (IGF-1), human growth hormone (HGH), and fetal bovine serum (FBS).

### Embodiment 74

The method of any one of embodiments 63-73, wherein the base or feed medium is Alpha MEM.

### Embodiment 75

A compound of Formula I

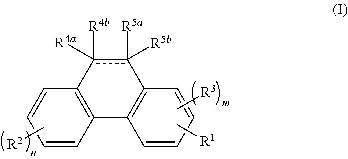

or a pharmaceutically acceptable salt, hydrate, or solvate thereof; wherein
the dashed line (represented by - - - - ) is an optional double bond; when either R^{4a} and R^{4b} or R^{5a} and R^{5b} combine to form an oxo or oxime moiety, the dashed line is a single bond;
R¹ is selected from the group consisting of —C(O)—NR^{b}—R^{1a}, —NR^{b}—C(O)—R^{1a}, —NR^{b}—C(O)—R^{1b}, —NR^{b}—X—C(O)—R^{1a}, —C(O)—X¹—NR^{b}—R^{1a}, —X¹—C(O)—NR^{b}—R^{1a}, —X¹—NR^{b}—C(O)—R^{1a}, —NR^{b}—C(O)—X¹—C(O)—R^{1b}, —C(O)—NR^{b}—X¹—C(O)—R^{1b}, —NR^{b}—C(O)—O—R^{1a}, —NR^{b}—C(O)—O—R^{1b}, —O—C(O)—NR^{b}—R^{1a}, —X¹—NR^{b}—C(O)—O—R^{1a}, —X¹—O—C(O)—NR^{b}—R^{1a}, —NR^{b}—R^{1a}, and —C(O)—R^{1a};
each R² is independently selected from the group consisting of halogen, —CN, —C₁₋₈ alkyl, —C₂₋₈ alkenyl, C₂₋₈ alkynyl, C₁₋₈ haloalkyl, —C₁₋₈ alkoxy, —X¹—C₁₋₈ alkoxy, —C(O)—R^{2a}, —SR^{a}, —X¹—SR^{a}, —OR^{a}, —X¹—OR^{a}, —NR^{a}R^{b}, —X¹—NR^{a}R^{b}, —S(O)₂R^{a}, —S(O)₂NR^{a}R^{b}, —X¹—S(O)₂R^{a}, and —X¹—S(O)₂NR^{a}R^{b}
each R³ is independently selected from the group consisting of halogen, —CN, —C₁₋₈ alkyl, —C₂₋₈ alkenyl, C₂₋₈ alkynyl, C₁₋₈ haloalkyl, —C₁₋₈ alkoxy, —X¹—C₁₋₈ alkoxy, —C(O)—R^{3a}, —SR^{a}, —X¹—SR^{a}, —OR^{a}, —X¹—OR^{a}, —NR^{a}R^{b}, —X¹—NR^{a}R^{b}, —S(O)₂R^{a}, —S(O)₂NR^{a}R^{b}, —X¹—S(O)₂R^{a}, and —X¹—S(O)₂NR^{a}R^{b};
R^{4a} is selected from the group consisting of —OR^{c} and —NR^{c}R^{d};
R^{4b} is H or absent; or R^{4a} and R^{4b} are combined to form an oxo moiety;
R^{5a} is selected from the group consisting of —OR^{c} and —NR^{c}R^{d};
R^{5b} is H or absent; or R^{5a} and R^{5b} are combined to an oxo moiety;
R^{1a} is selected from the group consisting of H, C₁₋₁₀ alkyl; C₁₋₁₀ haloalkyl;
R^{1b} is selected from the group consisting of —OR^{c}, —NR^{a}R^{b},
each R^{2a} and R^{3a} is independently selected from the group consisting of H, C₁₋₁₀ alkyl, C₁₋₁₀ haloalkyl, —OR^{a}, —X¹—OR^{a}, —NR^{a}R^{b}, and —X¹—NR^{a}R^{b};
each R^{a} and R^{b} is independently selected from the group consisting of H and C₁₋₄ alkyl;
each R^{c} and R^{d} is independently selected from the group consisting of H, C₁₋₈ alkyl, —C₂₋₈ alkenyl, C₂₋₈ alkynyl, C₁₋₈ haloalkyl, —C₁₋₈ alkoxy, —X¹—C₁₋₈ alkoxy, —SR^{a}, —X¹—SR^{a}, —OR^{a}, —X¹—OR^{a}, —NR^{a}R^{b}, —X¹—NR^{a}R^{b}, —C(O)—H, —C(O)—C₁₋₈alkyl, C₃₋₆ cycloalkyl, heterocycloalkyl, aryl, and heteroaryl;
each X¹ is independently C₁₋₄ alkylene;
the subscript n is an integer from 0 to 3; and
the subscript m is an integer from 0 to 2;
provided that the compound of Formula I is not N-(9,10-Dihydro-9,10-dioxo-2-phenanthrenyl)-2,2-dimethyl-propanamide.

### Embodiment 76

The compound of embodiment 75, wherein
R¹ is selected from the group consisting of —C(O)—NR^{b}—R^{1a}, —NR^{b}—C(O)—R^{1a}, —NR^{b}—X¹—C(O)—R^{1a}, —C(O)—X¹—NR^{b}—R^{1a}, —X¹—C(O)—NR^{b}—R^{1a}, —X¹—NR^{b}—C(O)—R^{1a}, —NR^{b}—C(O)—X¹—C(O)—R^{1b}, —C(O)—NR^{b}—X¹—C(O)—R^{1b}, —NR^{b}—C(O)—O—R^{1a}, —O—C(O)—NR^{b}—R^{1a}, —X¹—NR^{b}—C(O)—O—R^{1a}, and —X¹—O—C(O)—NR^{b}—R^{1a}.

### Embodiment 77

The compound of embodiment 75, wherein
R¹ is selected from the group consisting of —C(O)—NH—R^{1a}, —NH—C(O)—R^{1a}, —NH—X¹—C(O)—R^{1a}, —C(O)—X¹—NH—R^{1a}, —X¹—C(O)—NH—R^{1a}, —X¹—NH—C(O)—R^{a}, —NH—C(O)—X¹—C(O)—R^{1b}, —NH—C(O)—O—R^{a}, and —O—C(O)—NH—R^{1a}.

### Embodiment 78

The compound of embodiment 75, wherein
R¹ is selected from the group consisting of —C(O)—NH—R^{1a}, —NH—C(O)—R^{1a}, —NH—X¹—C(O)—R^{1a}, and —NH—C(O)—X¹—C(O)—R^{1b}.

### Embodiment 79

The compound of embodiment 75, wherein R¹ is —NH—C(O)—R^{1a}.

### Embodiment 80

The compound of any one of embodiments 75 to 79, wherein
each R² is independently selected from the group consisting of halogen, —CN, —C₁₋₈ alkyl, —C₂₋₈ alkenyl, C₂₋₈ alkynyl, C₁₋₈ haloalkyl, —C₁₋₈ alkoxy, —X¹—C₁₋₈ alkoxy, —C(O)—R^{2a}, —SR^{a}, —X¹—SR^{a}, —NR^{a}R^{b}, —X¹—NR^{a}R^{b}, —S(O)₂R^{a}, —S(O)₂NR^{a}R^{b}, —X¹—S(O)₂R^{a}, and —X¹—S(O)₂NR^{a}R^{b}.

### Embodiment 81

The compound of any one of embodiments 75 to 80, wherein
each R³ is independently selected from the group consisting of halogen, —CN, —C₁₋₈ alkyl, —C₂₋₈ alkenyl, C₂₋₈ alkynyl, C₁₋₈ haloalkyl, —C₁₋₈ alkoxy, —X¹—C₁₋₈ alkoxy, —C(O)—R^{3a}, —SR^{a}, —X¹—SR^{a}, —NR^{a}R^{b}, —X¹—NR^{a}R^{b}, —S(O)₂R^{a}, —S(O)₂NR^{a}R^{b}, —X¹—S(O)₂R^{a}, and —X¹—S(O)₂NR^{a}R^{b}.

### Embodiment 82

The compound of any one of embodiments 75 to 81, wherein
each R² and R³ is independently selected from the group consisting of halogen, —C₁₋₈ alkyl, C₁₋₈ haloalkyl, —C₁₋₈ alkoxy, —X¹—C₁₋₈ alkoxy, —NR^{a}R^{b}, —X¹—NR^{a}R^{b}, —S(O)₂R^{a}, —S(O)₂NR^{a}R^{b}, —X¹—S(O)₂R^{a}, and —X¹—S(O)₂NR^{a}R^{b}.

### Embodiment 83

The compound of any one of embodiments 75 to 81, wherein
each R² and R³ is independently selected from the group consisting of halogen, —C₁₋₈ alkyl, C₁₋₈ haloalkyl, —C₁₋₈ alkoxy, —X¹—C₁₋₈ alkoxy, —NR^{a}R^{b}, and —X¹—NR^{a}R^{b}, ^{b}.

### Embodiment 84

The compound of any one of embodiments 75 to 81, wherein
each R² and R³ is independently selected from the group consisting of halogen, —C₁₋₈ alkyl, C₁₋₈ haloalkyl, —NR^{a}R^{b}, and —X¹—NR^{a}R^{b}.

### Embodiment 85

The compound of any one of embodiments 75 to 81, wherein
each R² and R³ is —NR^{a}R^{b} or —X¹—NR^{a}R^{b}.

### Embodiment 86

The compound of any one of embodiments 75 to 85, wherein
R^{1a} is C₁₋₁₀ alkyl or C₁₋₁₀ haloalkyl.

### Embodiment 87

The compound of any one of embodiments 75 to 85, wherein
R^{1a} is C₁₋₆ alkyl or C₁₋₆ haloalkyl.

### Embodiment 88

The compound of any one of embodiments 75 to 85, wherein
R^{1b} is —OR^{a}.

### Embodiment 89

The compound of any one of embodiments 75 to 85, wherein
R^{1b} is —OH.

### Embodiment 90

The compound of any one of embodiments 75 to 89, wherein
each R^{a} and R^{b} is independently selected from the group consisting of H and C₁₋₂ alkyl.

### Embodiment 91

The compound of any one of embodiments 75 to 90, wherein
each X¹ is C₁₋₂ alkylene.

### Embodiment 92

The compound of any one of embodiments 75 to 90, wherein
each X¹ is C₁₋₂ alkylene.

### Embodiment 93

The compound of any one of embodiments 75 to 92, wherein
the subscript n is an integer from 1 to 3.

### Embodiment 94

The compound of any one of embodiments 75 to 92, wherein
the subscript n is 1.

### Embodiment 95

The compound of any one of embodiments 75 to 92, wherein
the subscript n is 0.

### Embodiment 96

The compound of any one of embodiments 75 to 95, wherein
the subscript m is an integer from 1 to 2.

### Embodiment 97

The compound of any one of embodiments 75 to 95, wherein
the subscript m is 0.

### Embodiment 98

The compound of any one of embodiments 75 to 95, wherein
the subscript m is 1.

### Embodiment 99

The compound of any one of embodiments 75 to 98, wherein the compound of Formula I has the structure of Formula I-1, I-2, I-3, or I-4

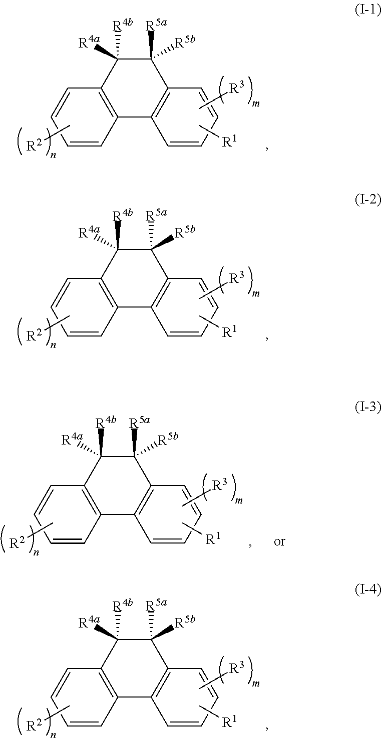

or a pharmaceutically acceptable salt, hydrate, or solvate thereof, wherein
R^{4a} is selected from the group consisting of —OR^{c}, and —NR^{c}R^{d};
R^{4b} is H;
R^{5a} is selected from the group consisting of —OR^{c}, and —NR^{c}R^{d};
R^{5b} is H.

### Embodiment 100

The compound of any one of embodiments 75 to 98, wherein the compound of Formula I has the structure of Formula Ia

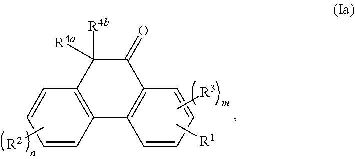

or a pharmaceutically acceptable salt, hydrate, or solvate thereof, wherein
R^{4a} is selected from the group consisting of —OR^{c}, and —NR^{c}R^{d};
R^{4b} is H.

### Embodiment 101

The compound of embodiment 100, wherein the compound of Formula Ia has the structure of Formula Ia1 or Ia2

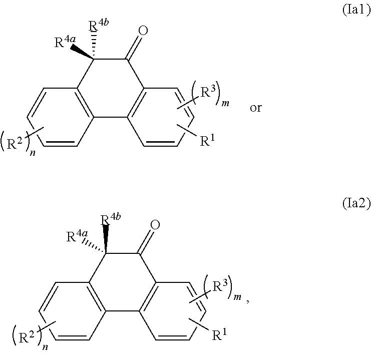

or a pharmaceutically acceptable salt, hydrate, or solvate thereof.

### Embodiment 102

The compound of any one of embodiment 75 to 98, wherein the compound of Formula I has the structure of Formula Ib

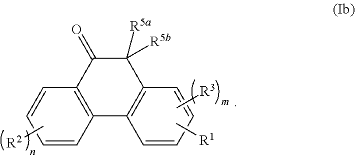

or a pharmaceutically acceptable salt, hydrate, or solvate thereof, wherein
R^{5a} is selected from the group consisting of —OR^{c}, and —NR^{a}R^{b};
R^{5b} is H.

### Embodiment 103

The compound of embodiment 102, wherein the compound of Formula Ib has the structure of Formula Ib1 or Ib2

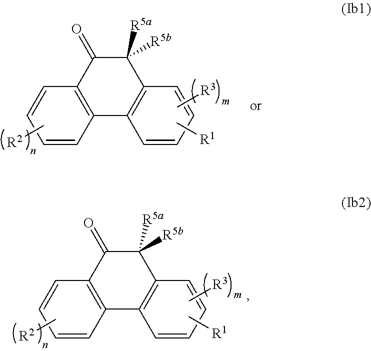

or a pharmaceutically acceptable salt, hydrate, or solvate thereof.

### Embodiment 104

The compound of any one of embodiments 99 to 103, wherein R^{4a} and R^{5a}, when present, are independently selected from the group consisting of —OH, —NH₂, —NH—C(O)—CH₃.

### Embodiment 105

The compound of any one of embodiments 99 to 103, wherein R^{4a} and R^{5a}, when present, are each —OH.

### Embodiment 106

A compound of any one of embodiments 75 to 98, wherein the compound of Formula I has the structure of Formula II

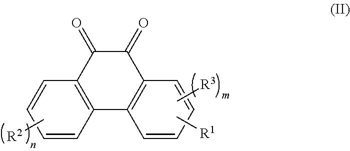

or a pharmaceutically acceptable salt, hydrate, or solvate thereof.

### Embodiment 107

The compound of embodiment 106, wherein the compound of Formula II has the structure of Formula IIa

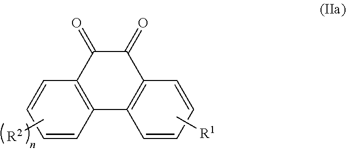

or a pharmaceutically acceptable salt, hydrate, or solvate thereof.

### Embodiment 108

The compound of embodiment 107, wherein
R¹ is selected from the group consisting of —C(O)—NH—R^{a}; —NH—C(O)—R^{1a}; —NH—X¹—C(O)—R^{a}; and —NH—C(O)—X¹—C(O)—R^{1b}
each R² is —NH₂;
R^{1a} is selected from the group consisting of C₁₋₁₀ alkyl; and C₁₋₁₀ haloalkyl;
R^{1b} is OH;
R^{a} and R^{b} are independently selected from the group consisting of H and C₁₋₄ alkyl;
X¹ is C₁₋₂ alkylene; and
the subscript n is an integer from 0 to 3;
provided that the compound of Formula II is not N-(9,10-Dihydro-9,10-dioxo-2-phenanthrenyl)-2,2-dimethyl-propanamide.

### Embodiment 109

The compound of any one of embodiments 106 to 108, wherein the compound of Formula II has the structure of Formula IIa1 or IIa2

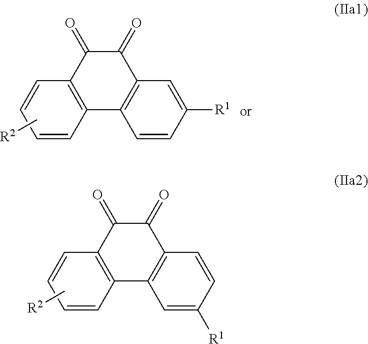

or a pharmaceutically acceptable salt, hydrate, or solvate thereof.

### Embodiment 110

The compound of any one of embodiments 106 to 108, where in the compound of Formula II has the structure of Formula IIb1 or Ib2

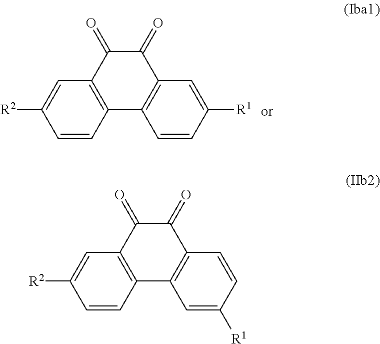

or a pharmaceutically acceptable salt, hydrate, or solvate thereof.

### Embodiment 111

The compound of embodiment 75, wherein said compound is selected from Table 1.

### Examples

The following examples are offered to illustrate, but not to limit the claimed invention.

Reagents and solvents used below can be obtained from commercial sources such as Signma-Aldrich Chemical Co. (Milwaukee, Wis., USA).

¹H-NMR spectra were recorded on a Varian Mercury 400 MHz NMR spectrometer. Chemical shifts were internally referenced to the residual proton resonance in CDCl3 (7.26 ppm) and are tabulated in the order: multiplicity (s, singlet; d, doublet; t, triplet; q, quartet; m, multiplet) and number of protons. ¹³C NMR was recorded at 100 MHz. Proton. Carbon chemical shifts were internally referenced to the deuterated solvent signals in CDCl3 (77.20 ppm).

Mass spectrometry results are reported as the ratio of mass over charge, followed by the relative abundance of each ion (in parenthesis). In the examples, a single m/z value is reported for the M+H (or, as noted, M−H) ion containing the most common atomic isotopes. Isotope patterns correspond to the expected formula in all cases. Electrospray ionization (ESI) mass spectrometry analysis was conducted on a Shimadzu LC-MS2020 using Agilent C18 column (Eclipse XDB-C18, 5 um, 2.1×50 mm) with flow rate of 1 mL/min. Mobile phase A: 0.1% of formic acid in water; mobile phase B: 0.1% of formic acid in acetonitrile. Normally the analyte was dissolved in methanol at 0.1 mg/mL and 1 microliter was infused with the delivery solvent into the mass spectrometer, which scanned from 100 to 1500 daltons. All compounds could be analyzed in the positive ESI mode, or analyzed in the negative ESI mode.

Analytical HPLC was performed on Agilent 1200 HPLC with a Zorbax Eclipse XDB C18 column (2.1×150 mm) with flow rate of 1 mL/min. Mobile phase A: 0.1% of TFA in water; mobile phase B: 0.1% of TFA in acetonitrile.

Preparative HPLC was performed on Varian ProStar using Hamilton C18 PRP-1 column (15×250 mm) with flow rate of 20 mL/min. Mobile phase A: 0.1% of TFA in water; mobile phase B: 0.1% of TFA in acetonitrile.

The following abbreviations are used in the Examples and throughout the description of the invention:
THF:—Tetrahydrofuran
TLC:—Thin layer chromatography
TFA:—Trifluoroacetic Acid
TEA:—Triethylamine
Tol:—Toluene
DCM: Dichloromethane
DCE:—1,2-dichloroethane
DMF:—Dimethyl formamide
DMSO: Dimethyl sulfoxide
DPPA: Diphenylphosphoryl azide
DIEA: N,N-Diisopropylethylamine
MeOH: Methanol
PE:—Petroleum ether
EA:—Etyl acetate
LCMS: Liquid Chomatography-Mass Spectrometry
HPLC: High Pressure Liquid Chromatography
HATU: 1-[Bis(dimethylamino)methylene]-1H-1,2,3-triazolo[4,5-b]pyridinium 3-oxid hexafluorophosphate
t-Bu:—tert-butyl
Et—Ethyl

Compounds within the scope of this invention can be synthesized as described below, using a variety of reactions known to the skilled artisan. One skilled in the art will also recognize that alternative methods may be employed to synthesize the target compounds of this invention, and that the approaches described within the body of this document are not exhaustive, but do provide broadly applicable and practical routes to compounds of interest.

Certain molecules claimed in this patent can exist in different enantiomeric and diastereomeric forms and all such variants of these compounds are within the scope of the present disclosure.

The detailed description of the experimental procedures used to synthesize key compounds in this text lead to molecules that are described by the physical data identifying them as well as by the structural depictions associated with them.

Those skilled in the art will also recognize that during standard work up procedures in organic chemistry, acids and bases are frequently used. Salts of the parent compounds are sometimes produced, if they possess the necessary intrinsic acidity or basicity, during the experimental procedures described within this patent.

### Example 1: Synthesis of N-(tert-butyl)-9,10-dioxo-9,10-dihydrophenanthrene-2-carboxamide (Compound 1.001)

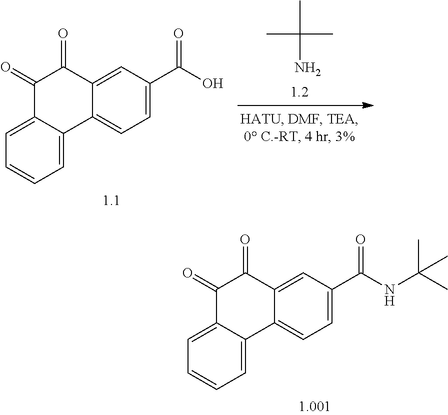

To a solution of Compound 1.1 (50 mg, 0.2 mmol, 1 eq) and Compound 1.2 (17.4 mg, 0.24 mmol, 1.2 eq) in dried THF (3 mL) was added HATU (90 mg, 0.238 mmol, 1.2 eq) and DIEA (51 mg, 0.4 mmol, 2 eq) at 0° C. under N₂. The mixture was warmed to rt, then stirred for 4 hr. The mixture was evaporated to give the residue. The residue was dissolved in EA, then, washed with water, dried over Na₂SO₄, and evaporated to give the crude product. The crude was purified via Prep-TLC to obtain Compound 1.001 (2 mg, yellow solid, 3%) LC-MS: 349 (M+41+1)⁺. ¹H NMR (400 MHz, CDCl₃): δ (ppm) 8.35 (d, J=2.1 Hz, 1H), 8.31-8.20 (m, 2H), 8.12 (d, J=8.4 Hz, 1H), 8.07 (d, J=8.0 Hz, 1H), 7.83-7.71 (m, 1H), 7.61-7.50 (m, 1H), 6.12 (s, 1H), 1.52 (d, J=0.7 Hz, 9H).

### Example 2: Synthesis of 2-((3,3-dimethyl-2-oxobutyl)amino)phenanthrene-9,10-dione (Compound 1.002)

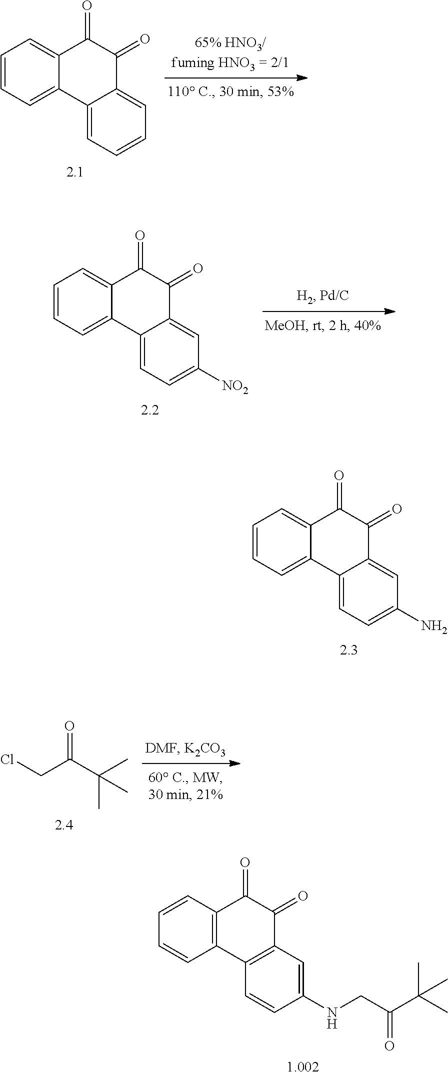

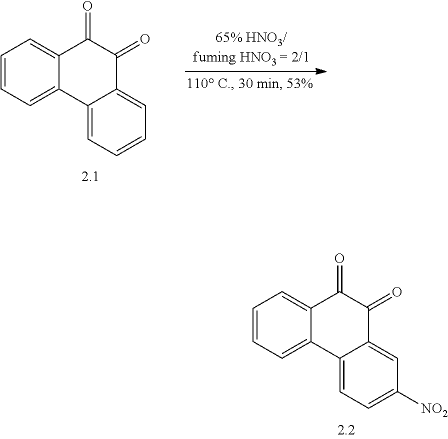 [SMILES: O=C1C(=O)c2ccccc2-c2ccccc21 ; O=C1C(=O)c2cc([N+](=O)[O-])ccc2-c2ccccc21]

To a mixture of compound 2.1 (15 g, 72 mmol, 1.0 eq) in 200 mL of concentrated HNO₃ (65%)/fuming HNO₃ (v/v=2/1) was heated quickly to boiling and kept at the boiling temperature for 30 minutes. The reaction was monitored by TLC. Then the mixture was poured into cool water and filtered to give the precipitate. The precipitate was dried under high vacuum. The yellow solid in AcOH (200 mL) was refluxed for 1 h. The mixture was cooled to 40-50° C., filtered, dried over Na₂SO₄ and concentrated to give compound 2.2 (9.5 g, 53%) as orange solid. TLC:PE:EA=8:1, UV 254 nm. Rf (compound 2.1)=0.5. Rf (compound 2.2)=0.3

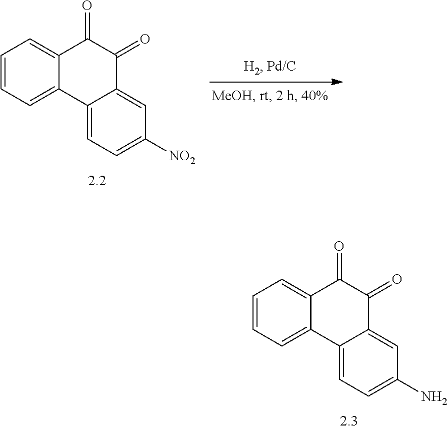 [SMILES: O=C1C(=O)c2cc([N+](=O)[O-])ccc2-c2ccccc21 ; Nc1ccc2c(c1)C(=O)C(=O)c1ccccc1-2]

To a mixture of compound 2.2 (9.5 g, 37.5 mmol, 1.0 eq) in MeOH (300 mL) was added Pd/C (4.3 g). The mixture was stirred at rt for 2 h under H₂. The reaction was monitored by TLC. Then the mixture was filtered and the filtrate was evaporated under rotary evaporation. The residue was purified by column chromatography on a silica gel (DCM/MeOH=10/1) to give 2.3 (3.4 g, 40%) as black solid. LCMS: [2M+Na]=469. ¹H NMR (400 MHz, DMSO): δ 7.97 (d, J=8.0 Hz, 1H), 7.90-7.84 (m, 2H), 7.63 (t, J=8.0 Hz, 1H), 7.29 (t, J=8.0 Hz, 1H), 7.18 (d, J=2.4 Hz, 1H), 6.90 (dd, J=8.0 and 2.4 Hz, 1H), 7.82-7.76 (m, 1H), 7.61-7.57 (m, 1H).

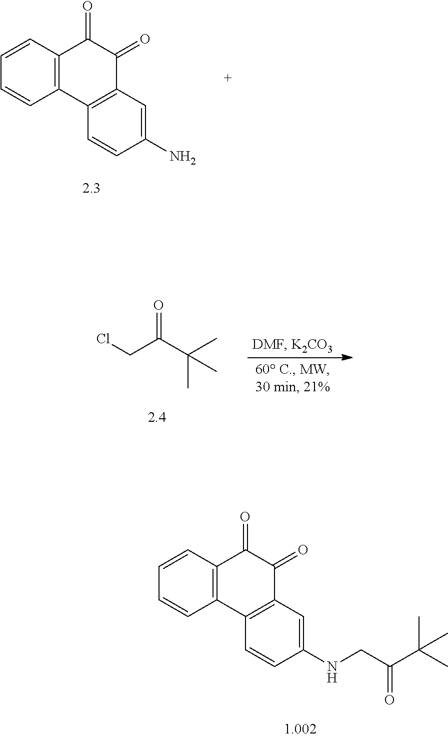

To a mixture of compound 2.3 (100 mg, 0.448 mmol, 1.0 eq), compound 2.4 (72.4 mg, 0.538 mmol, 1.2 eq) and K₂CO₃ (247.4 mg, 1.79 mmol, 4.0 eq) in dry DMF (5 mL) was stirred at 60° C. for 30 min in microwave. The reaction was monitored by LCMS. Then the mixture was filtered, added H₂O (5 mL), extracted with EA (3×5 mL). The organic layer was washed with brine. The residue was dried over Na₂SO₄, filtered and concentrated under reduced pressure. The residue was purified by Pre-HPLC to give Compound 1.002 (30 mg, 15%) as an orange solid. LCMS: [M+1]=322. ¹H NMR (400 MHz, (CD₃)₂SO): δ 8.02 (d, J=8.0 Hz, 1H), 7.87 (d, J=8.0 Hz, 1H), 7.69 (d, J=8.0 Hz, 2H), 7.35-7.25 (m, 1H), 6.71 (d, J=8.0 Hz, 1H), 6.54 (s, 1H), 4.29 (s, 2H), 0.92 (s, 9H).

### Example 3: Synthesis of 3-((7-amino-9,10-dioxo-9,10-dihydrophenanthren-2-yl)amino)-3-oxopropanoic acid (Compound 1.003)

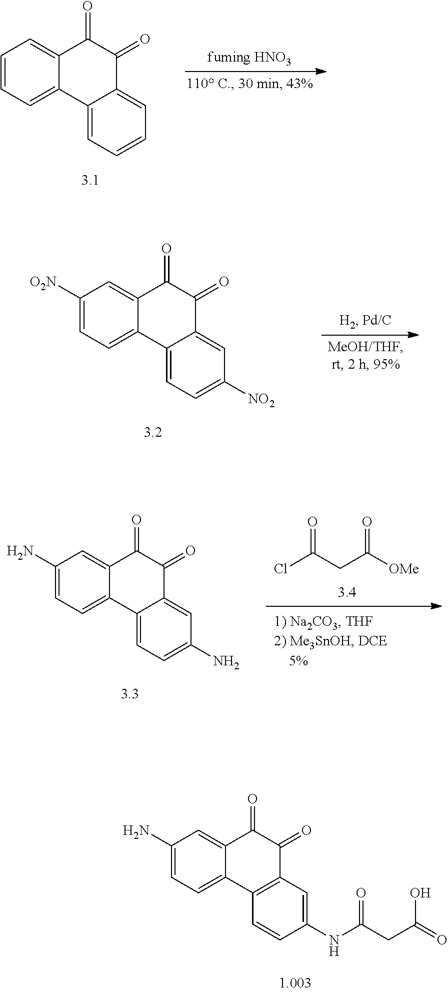 [SMILES: O=C1C(=O)c2ccccc2-c2ccccc21 ; O=C1C(=O)c2cc([N+](=O)[O-])ccc2-c2ccc([N+](=O)[O-])cc21 ; Nc1ccc2c(c1)C(=O)C(=O)c1cc(N)ccc1-2 ; COC(=O)CC(=O)Cl ; Nc1ccc2c(c1)C(=O)C(=O)c1cc(NC(=O)CC(=O)O)ccc1-2]

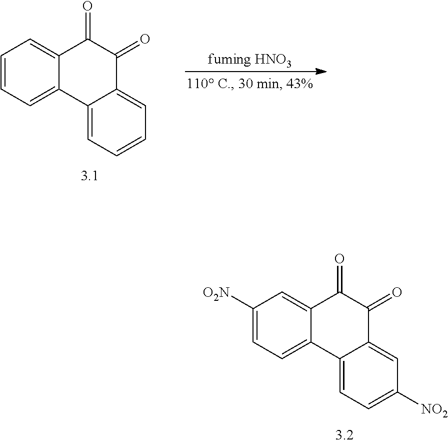 [SMILES: O=C1C(=O)c2ccccc2-c2ccccc21 ; O=C1C(=O)c2cc([N+](=O)[O-])ccc2-c2ccc([N+](=O)[O-])cc21]

To a mixture of compound 3.1 (7.0 g, 33.6 mmol, 1.0 eq) in 40 mL of fuming HNO₃ was heated quickly to boiling and kept at the boiling temperature for 30 minutes. The reaction was monitored by TLC. Then the mixture was poured into cool water and filtered to give the precipitate. The precipitate was dried under high vacuum. The yellow solid in AcOH (40 mL) was refluxed for 1 h. The mixture was cooled to 40-50° C., filtered, dried over Na₂SO₄ and concentrated to give compound 3.2 (4.3 g, 43%) as yellow solid.

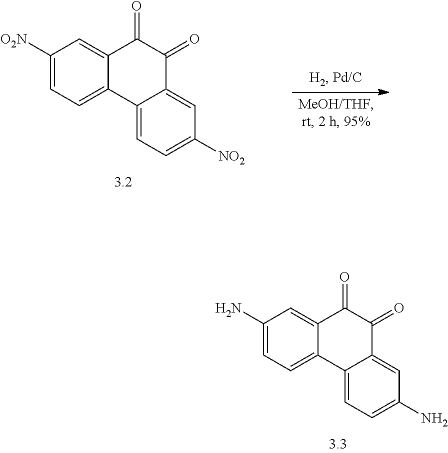 [SMILES: O=C1C(=O)c2cc([N+](=O)[O-])ccc2-c2ccc([N+](=O)[O-])cc21 ; Nc1ccc2c(c1)C(=O)C(=O)c1cc(N)ccc1-2]

To a mixture of compound 3.2 (4.0 g, 13.4 mmol, 1.0 eq) in MeOH (150 mL) was added Pd/C (1.0 g). The mixture was stirred at rt for 2 h under H₂. The reaction was monitored by TLC. Then the mixture was filtered and the filtrate was evaporated under rotary evaporation. The residue was purified by column chromatography on a silica gel (DCM/MeOH=10/1) to give compound 3.3 (3.2 g, 95%) as black solid. LCMS: [M+MeCN+1]: 280. ¹H NMR (400 MHz, DMSO): δ 7.71 (d, J=8.4 Hz, 2H), 7.20 (s, 2H), 6.95-6.85 (m, 2H).

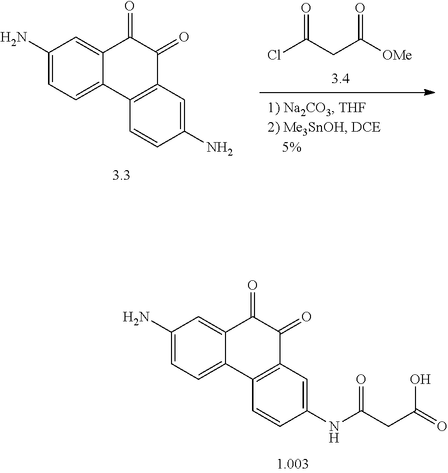 [SMILES: Nc1ccc2c(c1)C(=O)C(=O)c1cc(N)ccc1-2 ; COC(=O)CC(=O)Cl ; Nc1ccc2c(c1)C(=O)C(=O)c1cc(NC(=O)CC(=O)O)ccc1-2]

To a mixture of 3.3 (50 mg, 0.21 mmol, 1.0 eq) and Na₂CO₃ (88 mg, 0.84 mmol, 4.0 eq) in THF (2 mL) was added compound 3.4 (34.4 mg, 0.25 mmol, 1.2 eq) at 0° C. under nitrogen atmosphere. The mixture was stirred for 30 min. The reaction was monitored by LCMS. Then the mixture was quenched with H₂O, concentrated under reduced pressure. The mixture was diluted with DMF, filtered and concentrated under reduced pressure. The residue was purified by Pre-HPLC to give intermediate (12 mg). The intermediate (12 mg, 0.036 mmol, 1.0 eq) in DCE (2 mL) was added Me₃SnOH (22.5 mg, 0.12 mmol, 3.5 eq). The mixture was stirred at 85° C. overnight. The residue was purified by Prep-HPLC to give Compound 1.003 (3.3 mg, 5%) as a black solid. LCMS: [M+1]=325. ¹H NMR (400 MHz, (CD₃)₂SO): δ 10.35 (s, 1H), 8.12 (s, 1H), 7.80 (d, J=8.0 Hz, 1H), 7.85-7.75 (m, 3H), 7.25-7.10 (m, 2H), 6.95-6.85 (m, 1H), 2.75 (s, 2H).

### Example 4: Synthesis of N-(9,10-dioxo-9,10-dihydrophenanthren-2-yl)-3,3-dimethylbutanamide (Compound 1.004)

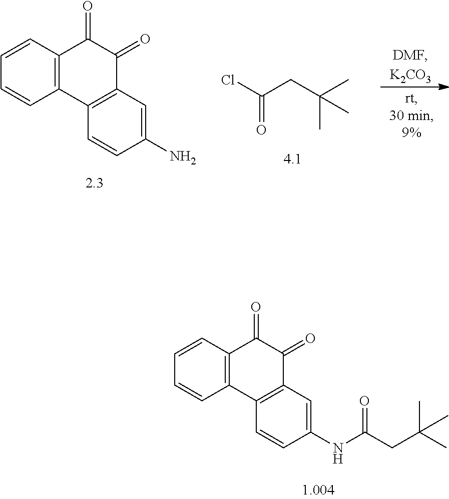

Compound 2.3 was prepared as described in Example 2. To a mixture of compound 2.3 (50 mg, 0.22 mmol, 1.0 eq) and Na₂CO₃ (95.1 mg, 0.9 mmol, 4.0 eq) in dry THF (2 mL) was added compound 4.1 (36.22 mg, 0.27 mmol, 1.2 eq) at 0° C. under nitrogen atmosphere. The mixture was stirred at rt for 30 min under nitrogen atmosphere. The reaction was monitored by LCMS. Then the mixture was quenched with H₂O, extracted with EA (3×2 mL). The organic layer was washed with brine. The residue was dried over Na₂SO₄, filtered and concentrated under reduced pressure. The residue was treated with EA and filtered to give Compound 1.004 (6.1 mg, 9%) as a black solid. LCMS: [M+1]=322. ¹H NMR (400 MHz, (CD₃)₂SO): δ 10.16 (s, 1H), 8.27 (s, 1H), 8.20-8.15 (m, 2H), 8.00-7.95 (m, 2H), 7.75-7.70 (m, 1H), 7.50-7.40 (m, 1H), 2.22 (s, 2H), 1.03 (s, 9H).

### Example 5: Synthesis of N-(9,10-dioxo-9,10-dihydrophenanthren-2-yl)decanamide (Compound 1.005)

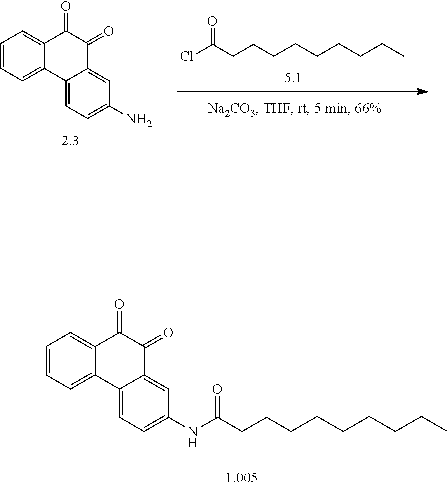 [SMILES: Nc1ccc2c(c1)C(=O)C(=O)c1ccccc1-2 ; CCCCCCCCCC(=O)Cl ; CCCCCCCCCC(=O)Nc1ccc2c(c1)C(=O)C(=O)c1ccccc1-2]

Compound 2.3 was prepared as described in Example 2. To a mixture of compound 2.3 (50 mg, 0.224 mmol, 1.0 eq) in THF (5 mL) was added Na₂CO₃ (95 mg, 0.896 mmol, 4.0 eq) and compound 5.1 (82 mg, 0.448 mmol, 2.0 eq). The mixture was stirred at rt for 5 min under nitrogen atmosphere. The reaction was monitored by TLC. Then the mixture was quenched with water (5 mL). The precipitated solid was filtered, washed with THF (5 mL). The solid was dried over Na₂SO₄, filtered and concentrated to give Compound 1.005 (56 mg, 66%) as red solid. TLC:PE:EA=2:1, UV 254 nm. Rf (2.3)=0.5. Rf (1.005)=0.6. LCMS: [M+42]: 419, [M−1]: 376. ¹H NMR (400 MHz, d6-DMSO): δ 10.22 (s, 1H), 8.27 (s, 1H), 8.25-8.16 (m, 2H), 8.00-7.93 (m, 2H), 7.72-7.69 (m, 1H), 7.47-7.43 (m, 1H), 2.35-2.31 (m, 2H), 1.59-1.57 (m, 2H), 1.30-1.20 (m, 12H), 0.85-0.82 (m, 3H).

### Example 6: Synthesis of N-(9,10-dioxo-9,10-dihydrophenanthren-2-yl)acetamide (Compound 1.006)

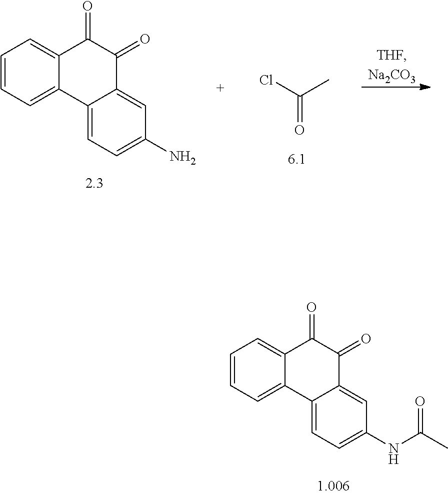

Compound 2.3 was prepared as described in Example 2. To a mixture of compound 2.3 (50 mg, 0.22 mmol, 1.0 eq) in THF (3 mL) was added Na₂CO₃ (93.3 mg, 0.88 mmol, 4.0 eq) and compound 6.1 (34 mg, 0.44 mmol, 2.0 eq). The mixture was stirred at rt for 1 h under nitrogen atmosphere. Water was added with stirring and the solid was filtered and washed with water to give Compound 1.006 (31 mg, 53%) as black solid. LCMS: [M−1]−=263. ¹H NMR (400 MHz, DMSO): δ 10.29 (s, 1H), 8.25-8.16 (m, 3H), 7.98-7.92 (m, 2H), 7.75-7.71 (m, 1H), 7.47-7.43 (m, 1H), 2.08 (s, 3H).

### Example 7: Synthesis of N-(7-amino-9,10-dioxo-9,10-dihydrophenanthren-2-yl)pivalamide (Compound 1.007)

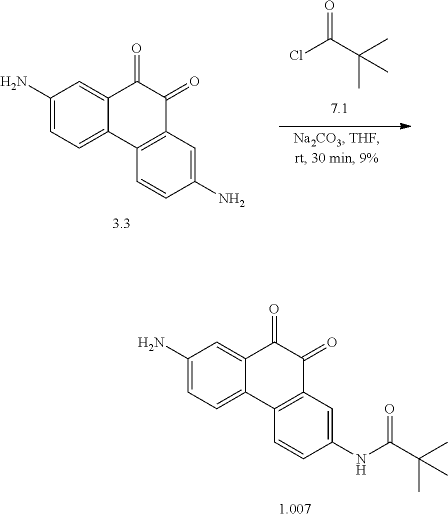

Compound 3.3 was prepared as described in Example 3. To a mixture of Compound 3.3 (50 mg, 0.21 mmol, 1.0 eq) and Na₂CO₃ (44.5 mg, 0.42 mmol, 2.0 eq) in dry THF (30 mL) was added compound 7.1 (28 mg, 0.23 mmol, 1.1 eq) at 0° C. under nitrogen atmosphere. The mixture was stirred at rt for 30 min under nitrogen atmosphere. The reaction was monitored by LCMS. Then the mixture was filtered, added H₂O (30 mL), extracted with EA (3×30 mL). The organic layer was washed with brine. The residue was dried over Na₂SO₄, filtered and concentrated under reduced pressure. The residue was purified by Prep-HPLC to give Compound 1.007 (6 mg, 9%) as a black solid. LCMS: [M+1]=323. ¹H NMR (400 MHz, (CD₃)₂SO): δ 9.42 (s, 1H), 8.17 (s, 1H), 8.00-7.75 (m, 3H), 7.15 (s, 1H), 6.90-6.85 (m, 1H), 5.74 (s, 2H), 1.22 (s, 9H).

### Example 8: Synthesis of N-(9,10-dioxo-9,10-dihydrophenanthren-2-yl)-2,2-dimethylbutanamide (Compound 1.008)

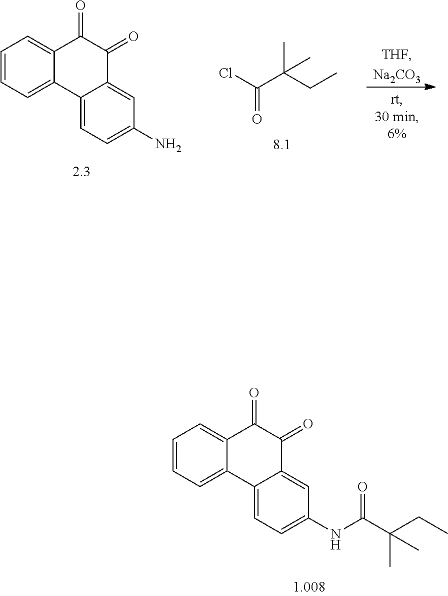

Compound 2.3 was prepared as described in Example 2. To a mixture of compound 2.3 (50 mg, 0.224 mmol, 1.0 eq) in THF (3 mL) was added Na₂CO₃ (95 mg, 0.896 mmol, 4.0 eq) and compound 8.1 (59 mg, 0.448 mmol, 2.0 eq). The mixture was stirred at rt for 30 min under nitrogen atmosphere. Then the mixture was filtered, added H₂O (3 mL), extracted with EA (3×3 mL). The residue was dried over Na₂SO₄ and concentrated under reduced pressure. The residue was purified by Pre-HPLC to give Compound 1.008 (4 mg, 6%) as yellow solid. LCMS: [M+1]=322. ¹H NMR (400 MHz, DMSO): δ 9.51 (s, 1H), 8.40 (s, 1H), 8.21-8.15 (m, 2H), 8.10-8.05 (m, 1H), 8.01-7.98 (m, 1H), 7.78-7.69 (m, 1H), 7.49-7.41 (m, 1H), 1.68-1.61 (m, 2H), 1.21 (s, 6H), 0.80-0.72 (m, 3H).

### Example 9: Synthesis of 2,2,2-trichloro-N-(9,10-dioxo-9,10-dihydrophenanthren-2-yl)acetamide (Compound 1.009)

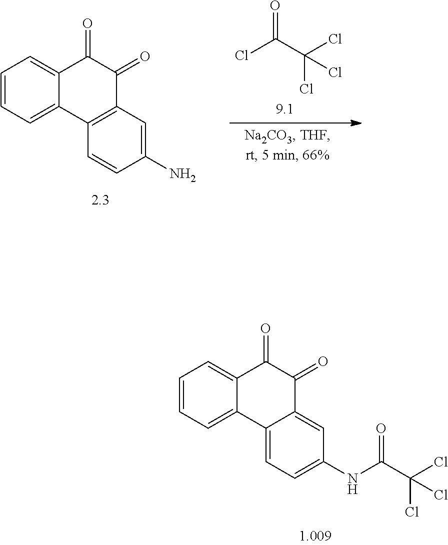 [SMILES: Nc1ccc2c(c1)C(=O)C(=O)c1ccccc1-2 ; O=C(Cl)C(Cl)(Cl)Cl ; O=C1C(=O)c2cc(NC(=O)C(Cl)(Cl)Cl)ccc2-c2ccccc21]

Compound 2.3 was prepared as described in Example 2. To a mixture of Compound 2.3 (50 mg, 0.224 mmol, 1.0 eq) in THF (5 mL) was added Na₂CO₃ (95 mg, 0.896 mmol, 4.0 eq) and compound 9.1 (81.6 mg, 0.448 mmol, 2.0 eq). The mixture was stirred at rt for 5 min under nitrogen atmosphere. The reaction was monitored by TLC. Then the mixture was filtered and concentrated. The precipitated solid was washed with water (10 mL), filtered, washed with DCM (3 mL). The solid was dried over Na₂SO₄, filtered and concentrated to give Compound 1.009 (54 mg, 66%) as red solid. TLC:PE:EA=2:1, UV 254 nm. Rf (compound 2.3)=0.5. Rf (Compound 1.009)=0.7. LCMS: [M−1]=367. ¹H NMR (400 MHz, d6-DMSO): δ 11.18 (s, 1H), 8.36-8.32 (m, 2H), 8.27-8.25 (m, 1H), 8.07-8.00 (m, 2H), 7.78-7.74 (m, 1H), 7.52-7.49 (m, 1H).

### Example 10: Synthesis of N-(9,10-dioxo-9,10-dihydrophenanthren-2-yl)-2,2-dimethylpentanamide (Compound 1.010)

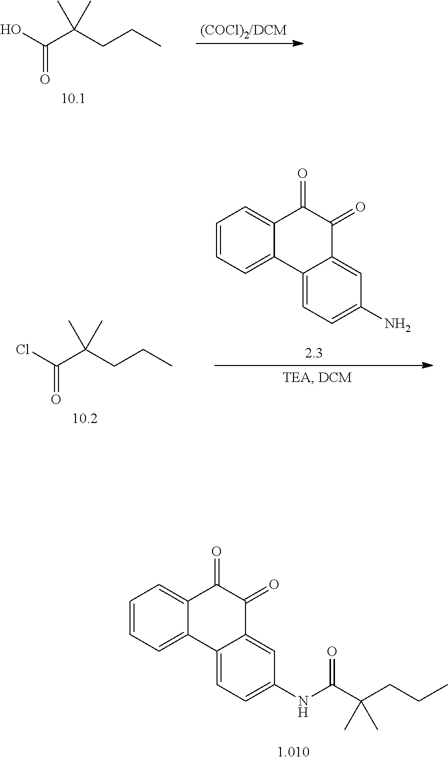

Compound 2.3 was prepared as described in Example 2. Compound 10.1 (50 mg, 0.38 mmol, 1.0 eq) was dissolved in DCM (5 mL) and cooled to 0° C. The solution was treated with oxalyl chloride (0.065 mL, 0.77 mmol, 2.0 eq), allowed to warm to RT, and stirred for 0.5 h for complete conversion to compound 10.2. The solution was cooled to 0° C. and added to a stirred solution of compound 2.3 (59 mg, 0.266 mmol, 0.7 eq) dissolved in DCM contains TEA (0.5 mL, 3.61 mmol, 10 eq) at 0° C. Stirring was continued at room temperature for 0.5 h. After all amines had been consumed as judged by TLC, the mixture was quenched with ice-water, extracted with DCM, dried over Na₂SO₄, concentrated and purified by Prep-TLC to give Compound 1.010 (6 mg, 5%) as red solid. LCMS: [M+1]⁺=336. ¹H NMR (400 MHz, DMSO): δ 9.52 (s, 1H), 8.31 (s, 1H), 8.22-8.18 (m, 2H), 8.10-8.07 (dd, J=8.8, 2.4 Hz, 1H), 7.99-7.97 (m, 1H), 7.73 (m, 1H), 7.47-7.45 (m, 1H), 1.60-1.56 (m, 2H), 1.24-1.19 (m, 8H), 0.87-0.84 (m, 3H).

### Example 11: Synthesis of 2,2,2-trichloro-N-(9,10-dioxo-9,10-dihydrophenanthren-3-yl)acetamide (Compound 1.011)

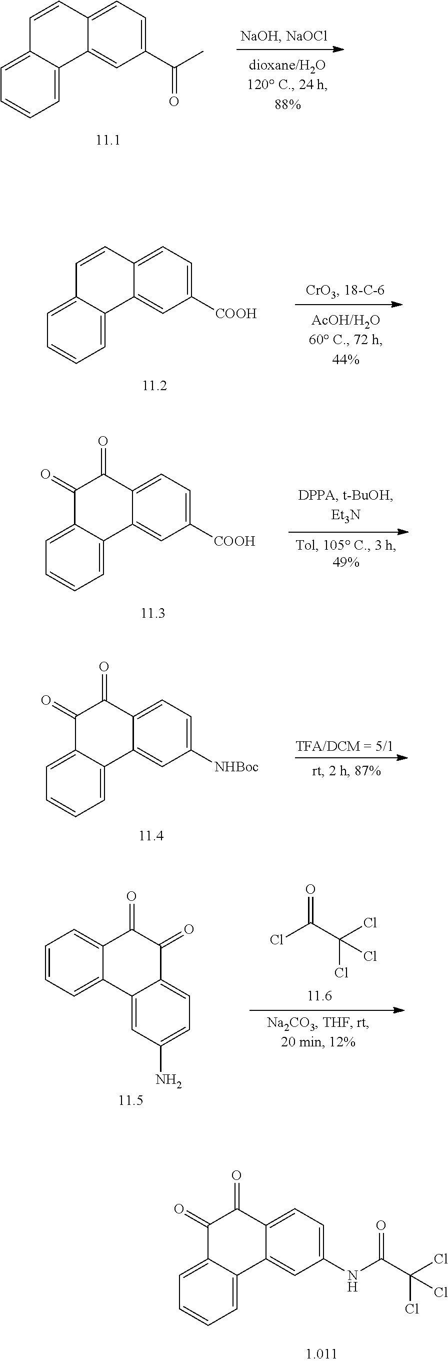

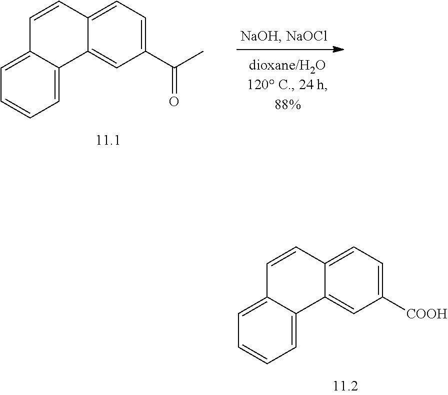 [SMILES: CC(=O)c1ccc2ccc3ccccc3c2c1 ; O=C(O)c1ccc2ccc3ccccc3c2c1]

To a mixture of compound 11.1 (4.5 g, 20.5 mmol, 1.0 eq) in dioxane/H₂O (120 mL/120 mL) was added NaOCl (140 mL, 205 mmol, 10.0 eq). The mixture was stirred at 100° C. for 24 h. The reaction was monitored by TLC. Then the mixture was cooled to rt and poured 1.5 L water, extorted with Et₂O (3×250 mL). The mixture was added HCl to adjust pH=2, filtered and washed with Et₂O. The residue was dried over Na₂SO₄ and concentrated to give compound 11.2 (4 g, 88%). TLC:PE:EA=5:1, UV 254 nm. Rf (compound 11.1)=0.8. Rf (compound 11.2)=0.05.

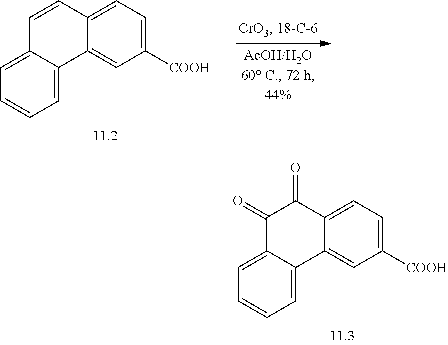 [SMILES: O=C(O)c1ccc2ccc3ccccc3c2c1 ; O=C(O)c1ccc2c(c1)-c1ccccc1C(=O)C2=O]

To a mixture of compound 11.2 (4.0 g, 18.02 mmol, 1.0 eq) in AcOH/H₂O (600 mL/30 mL) was added CrO₃ (5.4 g, 54.06 mmol, 3.0 eq) and 18-C-6 (0.95 g, 3.61 mmol, 0.2 eq). The mixture was stirred at 60° C. for 72 h. The reaction was monitored by HPLC. Then the mixture was added 1.0 L water, filtered. The solid was washed with AcOH:H₂O (1:1) (100 mL), washed with Et₂O (200 mL). The solid was dried over Na₂SO₄ and concentrated to give compound 11.3 (2 g, 44%) as red solid. LCMS: [M−1]=251. ¹H NMR (400 MHz, d6-DMSO): δ 8.69 (s, 1H), 8.32 (s, 1H), 8.05 (s, 1H), 8.02-7.98 (m, 2H), 7.77-7.73 (m, 1H), 7.58-7.54 (m, 1H).

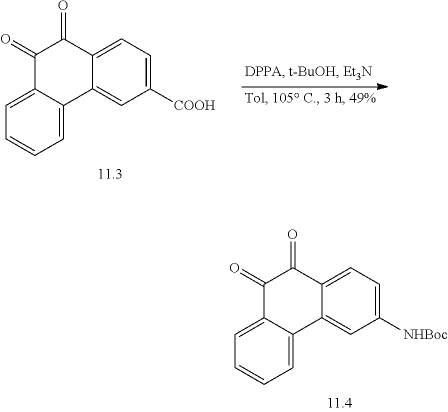 [SMILES: O=C(O)c1ccc2c(c1)-c1ccccc1C(=O)C2=O ; CC(C)(C)OC(=O)Nc1ccc2c(c1)-c1ccccc1C(=O)C2=O]

To a mixture of compound 11.3 (8.0 g, 31.7 mmol, 1.0 eq), Et₃N (5.2 g, 50.72 mmol, 1.6 eq) and t-BuOH (4.0 g, 53.89 mmol, 1.7 eq) in toluene (200 mL) was added DPPA (13.1 g, 47.55 mmol, 1.5 eq) at rt. The mixture was refluxed at 105° C. for 3 h. The reaction was monitored by LCMS. The reaction mixture was diluted with water (200 mL), filtered. The filtrate was extracted with EA (2×200 mL). The organic layers were combined washed with water (200 mL), brine (200 mL), decolorized with activated charcoal, dried, filtered and concentrated. The semisolid was crystallized from dichloromethane and hexanes to give compound 11.4 (5 g, 49%) as yellow solid. LCMS: [2M+Na]=669. ¹H NMR (400 MHz, DMSO): δ 8.42 (d, J=1.6 Hz, 1H), 8.18 (d, J=8.0 Hz, 1H), 8.14 (d, J=8.8 Hz, 1H), 8.03 (d, J=8.0 Hz, 1H), 7.70 (t, J=7.6 Hz, 1H), 7.47 (t, J=7.4 Hz, 1H), 7.17 (dd, J=8.4 and 2.0 Hz, 1H), 6.90 (s, 1H), 1.57 (s, 9H).

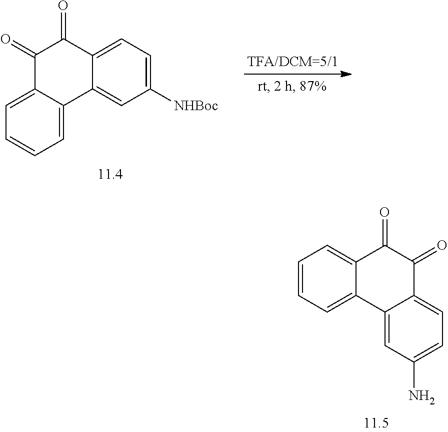 [SMILES: CC(C)(C)OC(=O)Nc1ccc2c(c1)-c1ccccc1C(=O)C2=O ; Nc1ccc2c(c1)-c1ccccc1C(=O)C2=O]

To a mixture of compound 11.4 (5 g, 15.5 mmol, 1.0 eq) in TFA/DCM (50 mL/10 mL) was stirred at rt for 2 h under nitrogen atmosphere. The reaction was monitored by TLC. Then the mixture was concentrated in vacuo, added water (20 mL) at 0° C. The mixture was added NaHCO₃ to adjust pH=7-8, extracted with DCM (3×50 mL). The organic layer was washed with brine. The residue was dried over Na₂SO₄ and concentrated under reduced pressure. The residue was purified by EA washing to give compound 11.5 (3 g, 87%) as black solid. TLC:PE:EA=1:1, UV 254 nm. Rf (compound 11.4)=0.6. Rf (11.6)=0.3. LCMS: [M+1]=224. ¹H NMR (400 MHz, DMSO): δ 7.99-7.94 (m, 2H), 7.82-7.74 (m, 2H), 7.51 (t, J=7.6 Hz, 1H), 7.28 (d, J=2.0 Hz, 1H), 6.85 (s, 2H), 6.62 (dd, J=8.8 and 2.0 Hz, 1H).

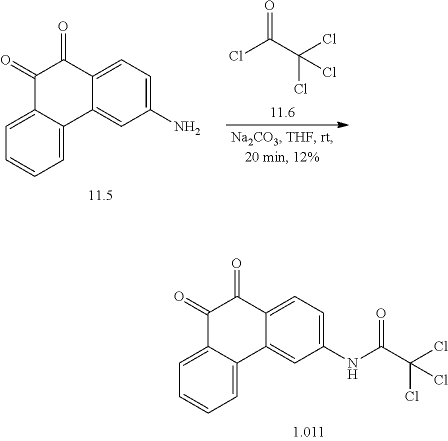 [SMILES: Nc1ccc2c(c1)-c1ccccc1C(=O)C2=O ; O=C(Cl)C(Cl)(Cl)Cl ; O=C1C(=O)c2ccc(NC(=O)C(Cl)(Cl)Cl)cc2-c2ccccc21]

To a mixture of compound 11.5 (50 mg, 0.224 mmol, 1.0 eq) in THF (3 mL) was added Na₂CO₃ (95 mg, 0.896 mmol, 4.0 eq) and compound 11.6 (82 mg, 0.448 mmol, 2.0 eq). The mixture was stirred at rt for 20 min under nitrogen atmosphere. The reaction was monitored by TLC. Then the mixture was filtered, added H₂O (3 mL), extracted with EA (3×3 mL). The residue was dried over Na₂SO₄ and concentrated under reduced pressure. The residue was purified by Pre-TLC to give Compound 1.011 (10 mg, 12%). LCMS: [M+1]=368. ¹H NMR (400 MHz, DMSO): δ 8.63 (d, J=2.0 Hz, 1H), 8.26-8.18 (m, 2H), 8.06 (d, J=8.4 Hz, 1H), 7.76-7.72 (m, 1H), 7.55-7.50 (m, 1H), 7.46 (dd, J=8.8 and 2.0 Hz, 1H).

### Example 12: Synthesis of 2-pivalamidophenanthrene-9,10-diyl Diacetate (Compound 1.012)

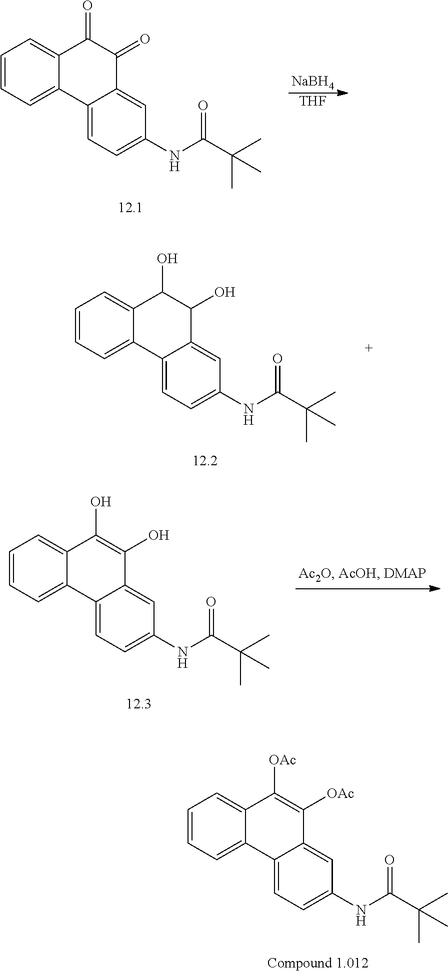 [SMILES: CC(C)(C)C(=O)Nc1ccc2c(c1)C(=O)C(=O)c1ccccc1-2 ; "NaBH4" ; CC(C)(C)C(=O)Nc1ccc2c(c1)C(O)C(O)c1ccccc1-2 ; CC(C)(C)C(=O)Nc1ccc2c(c1)c(O)c(O)c1ccccc12 ; "THF" ; CC(=O)Oc1c(OC(C)=O)c2cc(NC(=O)C(C)(C)C)ccc2c2ccccc12]

To a mixture of 12.1 (200 mg, 0.66 mmol, 1.0 eq) in THF (10 mL) was added NaBH₄ (100 mg, 2.6 mmol, 4.0 eq). The mixture was stirred at room temperature for 1 h. The reaction mixture was monitored by LCMS. The mixture was quenched with saturated aqueous NH₄Cl and extracted with EA. The organic layer was dried over Na₂SO₄ and concentrated under reduced pressure to give a mixture of 12.2 and 12.3 (200 mg) which was used in the next step without further purification.

To a mixture of 12.2 and 12.3 (200 mg, 0.643 mmol, 1.0 eq) in Ac₂O (3 mL) and AcOH (3 mL) was added catalytic amount of DMAP under nitrogen atmosphere. The mixture was stirred at 60° C. for 3 h. The reaction mixture was monitored by TLC. The mixture was diluted with EA and water, and the pH was adjusted to 8 with aqueous NaHCO₃. The organic layer was dried over Na₂SO₄ and concentrated under reduced pressure. The residue was purified by Prep-TLC (PE:EA=1:1) to give Compound 1.012 (98 mg, 38%) as a white solid. LCMS: [M+23]⁺=416. ¹H NMR (400 MHz, DMSO): δ 9.56 (s, 1H), 8.82 (t, J=8.4 Hz, 2H), 8.30 (s, 1H), 8.07 (d, J=7.6 Hz, 1H), 7.92 (d, J=7.6 Hz, 1H), 7.70 (m, 2H), 2.49 (m, 6H), 1.28 (s, 9H).

### Example 13: Synthesis of 3-hydroxyphenanthrene-9,10-dione (Compound 1.013)

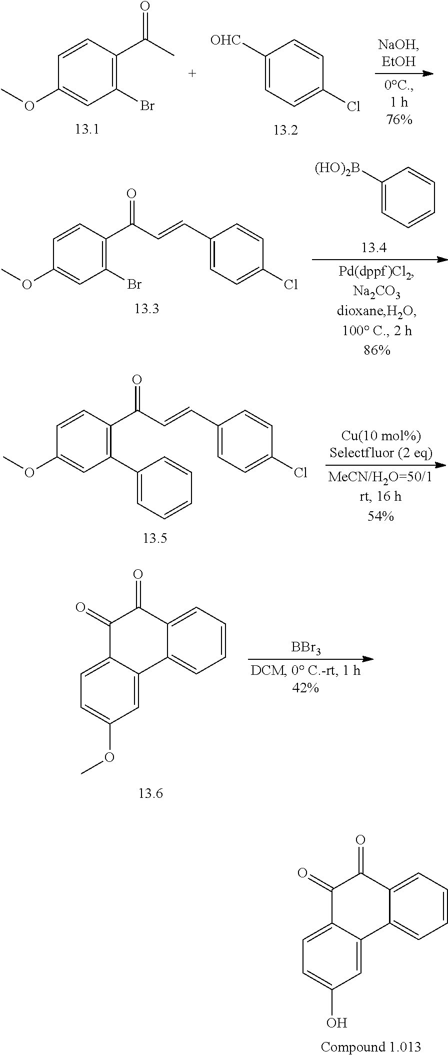

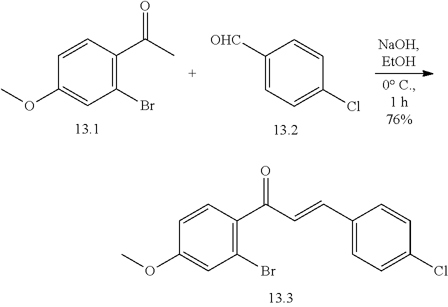 [SMILES: COc1ccc(C(C)=O)c(Br)c1 ; O=Cc1ccc(Cl)cc1 ; COc1ccc(C(=O)/C=C/c2ccc(Cl)cc2)c(Br)c1]

A mixture of compound 13.1 (10 g, 50.25 mmol, 1.0 eq) in EtOH/H₂O (100 mL/20 mL) was stirred at 0° C. for 5 min. Then compound 13.2 (7 g, 100.5 mmol, 2.0 eq) was added. The mixture was allowed to warm to room temperature and stirred at room temperature for 1 h. TLC analysis of the reaction mixture showed full conversion to the desired product. The reaction mixture was diluted with water and extracted with ethyl acetate. The organic layer was dried over anhydrous Na₂SO₄ and concentrated under reduced pressure. The residue was purified by silica gel chromatography to give compound 13.3 (11.7 g, 76%). TLC:PE:EA=15:1, 254 nm; R_{f} (13.1)=0.6; R_{f} (13.3)=0.5.

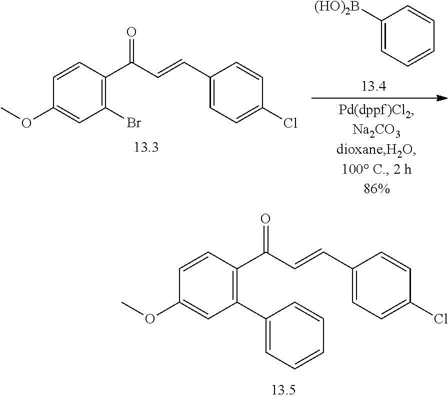 [SMILES: COc1ccc(C(=O)/C=C/c2ccc(Cl)cc2)c(Br)c1 ; OB(O)c1ccccc1 ; COc1ccc(C(=O)/C=C/c2ccc(Cl)cc2)c(-c2ccccc2)c1]

To a solution of compound 13.3 (11.7 g, 36.38 mmol, 1.0 eq), compound 13.4 (5.5 g, 36.38 mmol, 1.0 eq) and Na₂CO₃ (11.6 g, 109.1 mmol, 3.0 eq) in dioxane/H₂O (100 mL/10 mL) was added Pd(dppf)Cl₂ (3 g, 3.64 mmol, 0.1 eq) under nitrogen atmosphere. The mixture was stirred at 100° C. for 2 h. TLC analysis of the reaction mixture showed full conversion to the desired product. Then the mixture was filtered through a pad of celite and sintered funnel. The reaction mixture was diluted with water and extracted with ethyl acetate. The organic layer was dried over anhydrous Na₂SO₄ and concentrated under reduced pressure. The residue was purified by silica gel chromatography (PE: EA=100%-30:1) to afford compound 13.5 (10 g, 86%). TLC:PE:EA=15:1, 254 nm; R_{f} (13.3)=0.5; R_{f} (13.5)=0.4.

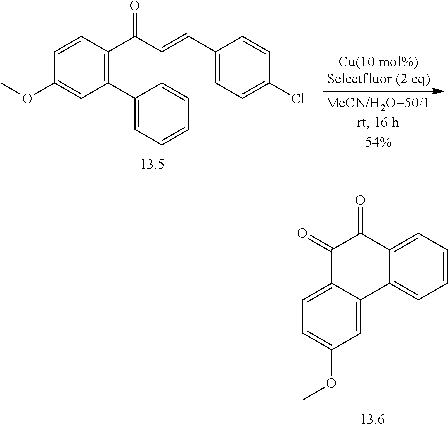 [SMILES: COc1ccc(C(=O)/C=C/c2ccc(Cl)cc2)c(-c2ccccc2)c1 ; COc1ccc2c(c1)-c1ccccc1C(=O)C2=O]

To a solution of compound 13.5 (1 g, 2.87 mmol, 1.0 eq) in ACN/H₂O (10 mL/0.2 mL) was added Cu powder (18.5 mg, 0.287 mmol, 0.1 eq) and Selectfluor (2.03 g, 5.75 mmol, 2.0 eq). The mixture was stirred at room temperature for 12 h. TLC analysis of the reaction mixture showed full conversion to the desired product. The reaction mixture was diluted with water and extracted with ethyl acetate. The organic layer was dried over anhydrous Na₂SO₄ and concentrated under reduced pressure. The residue was purified by silica gel chromatography to afford crude compound 13.6 (370 mg, 54%). TLC:PE:EA=6:1, 254 nm; R_{f} (13.5)=0.8; R_{f} (13.6)=0.2.

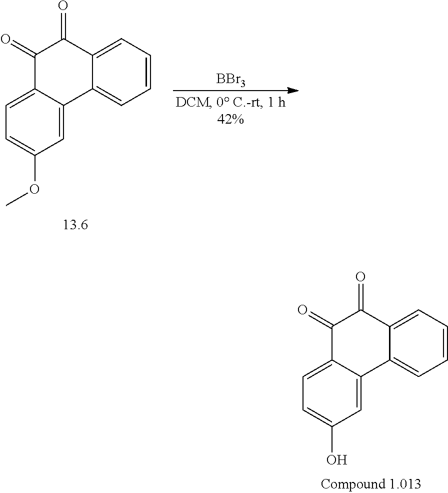

To a solution of compound 13.6 (30 mg, 0.126 mmol, 1.0 eq) in DCM (5 mL) was slowly added BBr₃ (2 M in DCM, 0.13 mL, 0.252 mmol, 2.0 eq) at 0° C. After addition, the mixture was stirred for 15 min under nitrogen atmosphere. LCMS analysis of the reaction mixture showed full conversion to the desired product. Then the mixture was concentrated under reduced pressure and the residue was purified by prep-HPLC to afford compound 1.013 (12 mg, 42%) as a yellow solid. LCMS: [M+1]=225; ¹H NMR (400 MHz, CD₃OD) δ 8.16 (d, J=8.0 Hz, 1H), 8.01 (dd, J=7.7, 1.3 Hz, 1H), 7.95 (d, J=8.6 Hz, 1H), 7.79 (td, J=7.9, 1.5 Hz, 1H), 7.58-7.51 (m, 2H), 6.91 (dd, J=8.6, 2.2 Hz, 1H).

### Example 14: Synthesis of N-(10-hydroxy-9-(2-methoxyethoxy)phenanthren-2-yl)pivalamide (Compound 1.014)

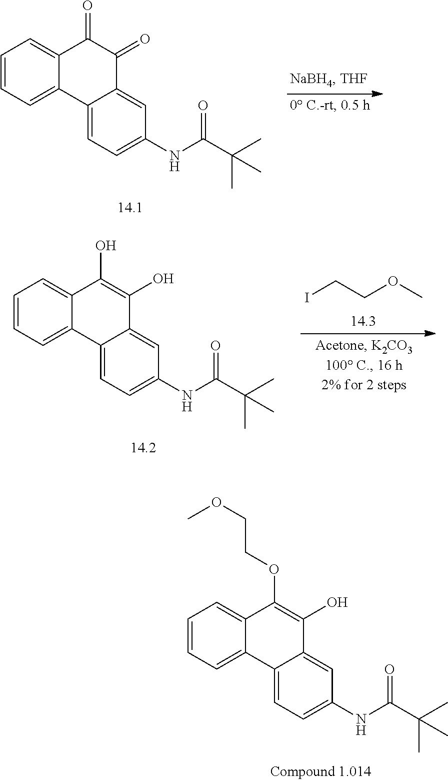

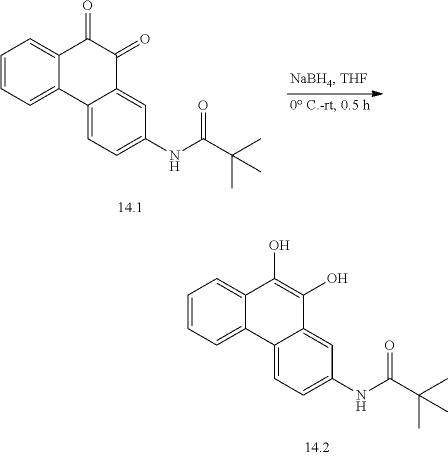 [SMILES: CC(C)(C)C(=O)Nc1ccc2c(c1)C(=O)C(=O)c1ccccc1-2 ; CC(C)(C)C(=O)Nc1ccc2c(c1)c(O)c(O)c1ccccc12]

To a solution of compound 14.1 (120 mg, 0.391 mmol, 1.0 eq) in THF (2 mL) was added NaBH₄ (59 mg, 1.56 mmol, 4.0 eq) at 0° C. under nitrogen atmosphere. The mixture was allowed to warm to room temperature and stirred at room temperature for 0.5 h. TLC analysis of the reaction mixture showed full conversion to the desired product (the color of the mixture was light from yellow). Then the mixture was quickly quenched with saturated aqueous of NaHCO₃ and extracted with ethyl acetate under nitrogen atmosphere (when the product is exposed to air, it will turn back to the raw material). The organic layer was quickly filtered through a pad of anhydrous Na₂SO₄ and sintered funnel under nitrogen atmosphere and concentrated under reduced pressure to afford crude compound 14.2, which was used for next step directly under nitrogen atmosphere.

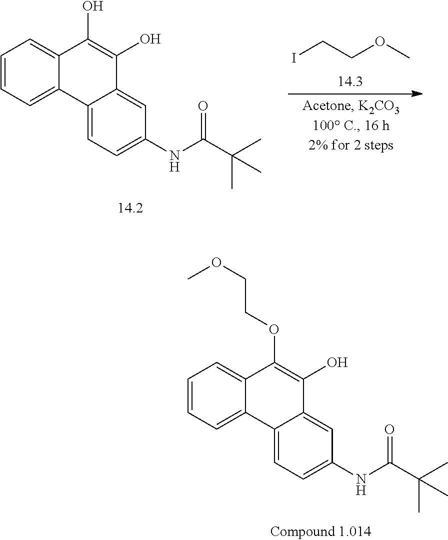

To a solution of compound 14.2 from previous step in acetone (2 mL) was successively added compound 14.3 (0.5 mL) and K₂CO₃ (162 mg, 1.17 mmol, 3.0 eq) under nitrogen atmosphere in a sealed tube. The resulting mixture was stirred at 100° C. for 16 h under nitrogen atmosphere. LCMS analysis of the reaction mixture showed full conversion to the desired product. The reaction mixture was diluted with water (10 mL) and extracted with EA (3*10 mL). The combined organic phases were dried over anhydrous Na₂SO₄ and concentrated under reduced pressure. The residue was purified by prep-HPLC to afford compound Compound 1.014 (3 mg, 2%) as a yellow solid. ¹H NMR (400 MHz, CDCl₃): δ 8.50-7.30 (m, 7H), 4.37-4.25 (m, 4H), 3.62 (s, 3H), 1.35 (s, 9H).

### Example 15: Synthesis of 2-(benzyloxy)-N-(9,10-dioxo-9,10-dihydrophenanthren-2-yl)acetamide (Compound 1.015)

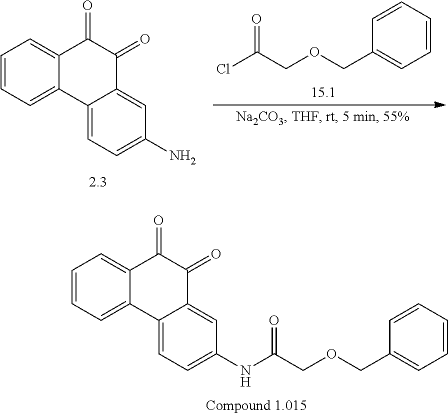 [SMILES: Nc1ccc2c(c1)C(=O)C(=O)c1ccccc1-2 ; O=C(Cl)COCc1ccccc1 ; O=C(COCc1ccccc1)Nc1ccc2c(c1)C(=O)C(=O)c1ccccc1-2]

Compound 2.3 was prepared as described in Example 2. To a mixture of compound 2.3 (50 mg, 0.224 mmol, 1.0 eq) in THF (5 mL) was added Na₂CO₃ (95 mg, 0.896 mmol, 4.0 eq) and compound 15.1 (82 mg, 0.448 mmol, 2.0 eq). The mixture was stirred at rt for 5 min under nitrogen atmosphere. The reaction was monitored by TLC. Then the mixture was quenched with water (5 mL). The precipitated solid was filtered, washed with THF (5 mL). The solid was dried over Na₂SO₄, filtered and concentrated to give Compound 1.015 (46 mg, 55%) as red solid. TLC:PE:EA=2:1, UV 254 nm; Rf (compound 15.1)=0.5; Rf (Compound 1.015)=0.6. LCMS: [M−1]=370. ¹H NMR (400 MHz, d6-DMSO): δ 10.20 (s, 1H), 8.36 (s, 1H), 8.25-8.16 (m, 2H), 8.03-7.97 (m, 2H), 7.73-7.49 (m, 1H), 7.50-7.30 (m, 6H), 4.63 (s, 2H), 4.13 (s, 2H).

### Example 16: Synthesis of N-(9-(hydroxyimino)-10-oxo-9,10-dihydrophenanthren-2-yl)pivalamide (Compound 1.016) & (E)-N-(10-(hydroxyimino)-9-oxo-9,10-dihydrophenanthren-2-yl)pivalamide (Compound 1.017)

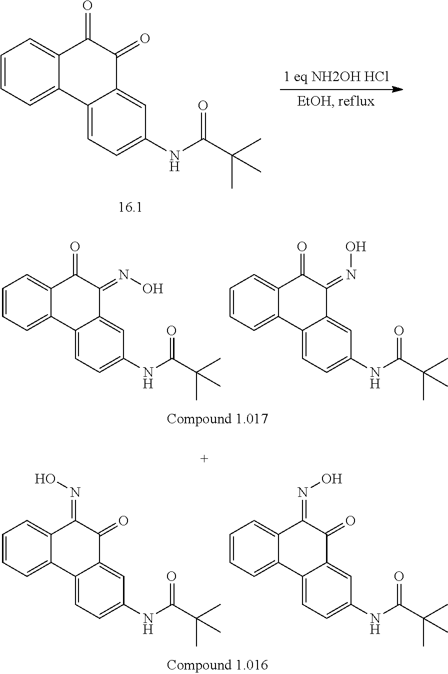 [SMILES: CC(C)(C)C(=O)Nc1ccc2c(c1)C(=O)C(=O)c1ccccc1-2 ; CC(C)(C)C(=O)Nc1ccc2c(c1)/C(=N\O)C(=O)c1ccccc1-2 ; CC(C)(C)C(=O)Nc1ccc2c(c1)/C(=N/O)C(=O)c1ccccc1-2 ; CC(C)(C)C(=O)Nc1ccc2c(c1)C(=O)/C(=N/O)c1ccccc1-2 ; CC(C)(C)C(=O)Nc1ccc2c(c1)C(=O)/C(=N\O)c1ccccc1-2]

A mixture of 16.1 (600 mg, 1.95 mmol) and hydroxylamine. HCl (136 mg, 1.95 mmol) in EtOH (10 mL) was stirred for 6 hours at reflux. After the mixture was cooled to rt, EtOAc (40 mL) was added. The organics were washed with water (2×30 mL) and brine (30 mL), dried under Na₂SO₄ and concentrated to give a mixture of Compound 1.016 and Compound 1.017 (610 mg, 97%). The mixture (40 mg) was purified by prep-HPLC to give Compound 1.016 (9 mg, 23%) and Compound 1.017 (9 mg, 23%). Compound 1.016: LC-MS: 323[M+H]. ¹H NMR (CDCl₃): δ 8.37-8.22 (m, 2H), 8.11-8.01 (m, 3H), 7.76 (t, J=6.3 Hz, 1H), 7.55-7.21 (m, 3H), 1.37, 1.36 (s, 9H). Compound 1.017: LC-MS: 323[M+H]. ¹H NMR (CDCl₃): δ 8.36-8.20 (m, 2H), 8.11-7.99 (m, 3H), 7.75 (t, J=6.6 Hz, 1H), 7.60-7.91 (m, 3H), 1.37, 1.36 (s, 9H).

### Example 17: Isolation and Enhancement of Hematopoietic Stem Cells Derived from Non-Mobilized Peripheral Blood Using SF1670 or SF1670 and ER50891 in Culture

This Example describes the isolation and culturing of hematopoietic stem cells derived from non-mobilized peripheral blood. This Example also demonstrates the enhancement of HSCs in cultures containing SF1670 or SF1670 and ER50891.

### Materials and Methods

CD34+ cells were isolated from donor peripheral blood. Whole blood was centrifuged at 1750×g (the speed of centriguation may vary) for 20 minutes. Plasma was drawn off using a pipette or syringe. Concentrated red blood cell (RBC) layer was drawn off with a pipette or syringe. Residual RBCs were lysed using standard RBC lysis protocols. White blood cell (WBC) layer was washed in PBS and spun down several times at 300 g for 7 minutes to remove platelets and some debris. Cells were pelleted and incubated with unlabeled CD64 antibody. Cells then undergo negative depletion using biotinylated CD2, CD3, CD4, CD5, CD8, CD11b, CD14, CD16, CD19, CD20, CD45RA, CD56, CD235 (in some examples CD15, CD25 and other lineage specific antibodies may also be used). Cells which bind these antibodies are depleted using streptavidin beads. The resultant progenitor enriched cell pool were cultured directly (residual mature cells and late progenitors will be killed off over time in culture) or cell pool can undergo a CD34 or CD133 positive selection to further enrich for the desired cells before culturing.

Isolated CD34+ cells were incubated in an in vitro culture media of Alpha MEM without phenol red, 10% (v/v) heat inactivated fetal bovine serum (FBS). Base Conditions, +SF Conditions, and +SF/+ER conditions further included the components described in Table 2. Each condition tested also included an antibiotic solution that includes penicillin, streptomycin, and amphotericin B to avoid contamination.

TABLE 2
Additional Components included in the culture media of Base Conditions,
+SF Conditions, and +SF/+ER Conditions.

| | - Factor - | - Concentration - | |
|---|---|---|---|
| Base | Cytokines/Growth Factors | | |
| Conditions | TPO | 100 | ng/mL |
| | SCF | 100 | ng/mL |
| | FLT3L | 100 | ng/mL |
| +SF | Cytokines/Growth Factors | | |
| Conditions | TPO | 100 | ng/mL |
| | SCF | 100 | ng/mL |
| | FLT3L | 100 | ng/mL |
| | Small Molecules | | |
| | SF1670 (PTEN inhibitor) | 250 | nM |
| +SF/+ER | Cytokines/Growth Factors | | |
| Conditions | TPO | 100 | ng/mL |
| | SCF | 100 | ng/mL |
| | FLT3L | 100 | ng/mL |

| Small Molecules | | |
|---|---|---|
| ER50891 (RAR receptor | 100 | nM |
| antagonist) | | |
| SF1670 (PTEN inhibitor) | 250 | nM |

Cultures were incubated at 3% oxygen (controlled by nitrogen) and 5% CO₂.

Small molecule components were added separately and fresh each time the media needs to be refreshed. Cytokines can be stored together. Media renewal should occur at least every few days.

On the days indicated one-half of the volume of the cell culture was removed for data analysis (flow cytometry using a BD FACS ARIA II). The culture volume was replenished with fresh media according to the conditions tested. The data reported accounts for the dilution factor introduced in this procedure.

Results

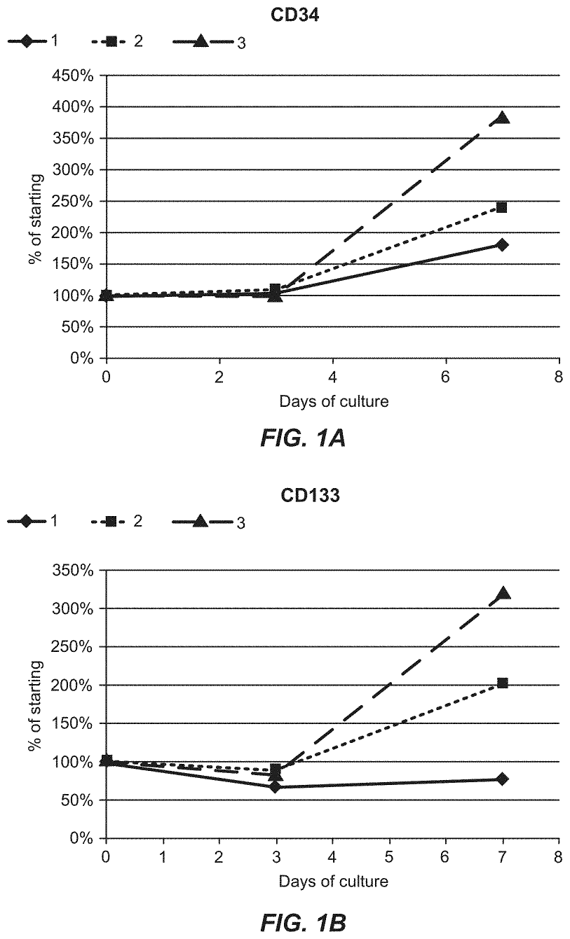

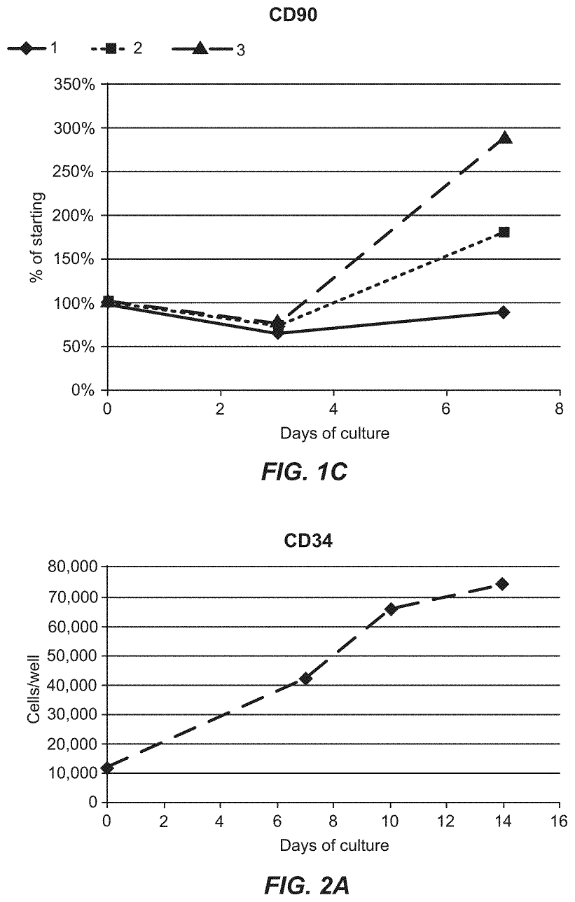

Flow cytometric analysis of 3 and 7 day cell cultures as described above demonstrates that addition of SF1670 or SF1670 and ER50891 maintains and improves the expansion of hematopoietic stem cells (FIG. 1A-C). +SF Conditions and +SF/+ER Conditions provide increased numbers of CD34+ cells (FIG. 1A), CD133+ cells (FIG. 1B), and CD90+(FIG. 1C) after 7 days in culture.

### Example 18: Addition of SF1670 and ER50891 to Culture Media Improves the Maintenance and Expansion of Hematopoietic Stem Cells

This Example demonstrates the enhancement of HSCs in cultures containing SF1670 and ER50891. The number of HSCs in culture continues to increase through 14 days of in vitro incubation.

### Materials and Methods

CD34+ cells were isolated from donor peripheral blood as described in Example 17. Isolated CD34+ cells were incubated in an in vitro culture media of Alpha MEM without phenol red, 10% (v/v) heat inactivated fetal bovine serum (FBS) and +SF/+ER conditions. +SF+ER conditions included the components described in Table 3. The culture also included an antibiotic solution that includes penicillin, streptomycin, and amphotericin B to avoid contamination.

TABLE 3
Additional Components included in the culture
media of +SF/+ER Conditions.

| | - Factor - | - Concentration - | |
|---|---|---|---|
| +SF/+ER | Cytokines/Growth Factors | | |
| Conditions | TPO | 100 | ng/mL |
| | SCF | 100 | ng/mL |
| | FLT3L | 100 | ng/mL |

| Small Molecules | | |
|---|---|---|
| ER50891 (RAR receptor | 100 | nM |
| antagonist) | | |
| SF1670 (PTEN inhibitor) | 250 | nM |

Cultures were incubated at 3% oxygen (controlled by nitrogen) and 5% CO₂.

Small molecule components were added separately and fresh each time the media needs to be refreshed. Cytokines can be stored together. Media renewal should occur at least every few days.

On the days indicated one-half of the volume of the cell culture was removed for data analysis (flow cytometry using a BD FACS ARIA II). The culture volume was replenished with fresh media according to the conditions tested. The data reported accounts for the dilution factor introduced in this procedure.

Results

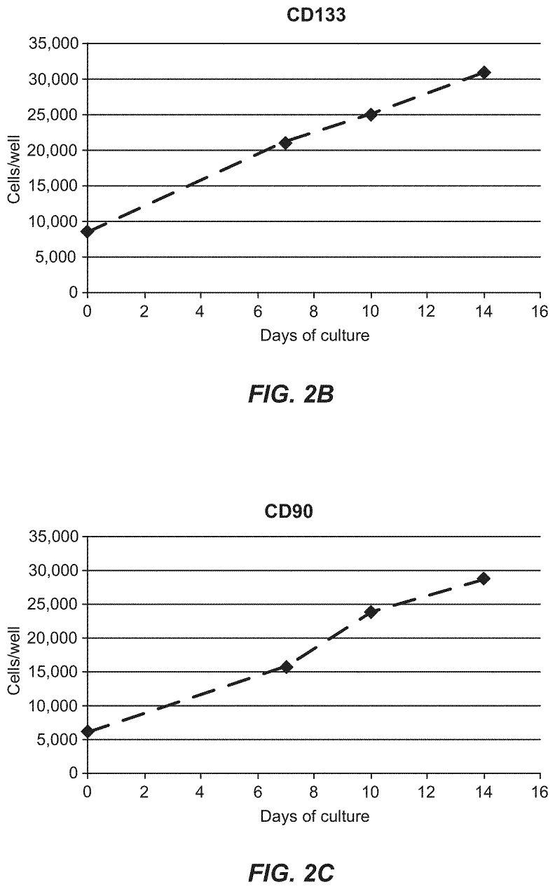

Flow cytometric analysis of +SF/+ER Conditions demonstrates that hematopoietic stem cells are maintained and continue to expand even after 14 days in culture (FIG. 2A-C). Indeed, after 14 days in culture+SF/+ER Conditions provide increased numbers of CD34+ cells (FIG. 2A), CD133+ cells (FIG. 2B), and CD90+(FIG. 2C).

### Example 19: Enhancement of Hematopoietic Stem Cells Derived from Cord Blood Using SF1670 in Culture

This Example describes the isolation and culturing of hematopoietic stem cells derived from cord blood. The number of HSCs in culture continues to increase through 19 days of in vitro incubation.

### Materials and Methods

A frozen cord blood sample was thawed and gradually brought to room temperature. Thawed cord blood was incubated in an in vitro culture media of Alpha MEM without phenol red, 10% (v/v) heat inactivated fetal bovine serum (FBS). Base Conditions and +SF Conditions further included the components described in Table 4. Each condition tested also included an antibiotic solution that includes penicillin, streptomycin, and amphotericin B to avoid contamination.

TABLE 4
Additional Components included in the culture
media of Base Conditions, +SF Conditions.

| | - Factor - | - Concentration - | |
|---|---|---|---|
| Base | Cytokines/Growth Factors | | |
| Conditions | TPO | 100 | ng/mL |
| | SCF | 100 | ng/mL |
| | FLT3L | 100 | ng/mL |
| +SF | Cytokines/Growth Factors | | |
| Conditions | TPO | 100 | ng/mL |
| | SCF | 100 | ng/mL |
| | FLT3L | 100 | ng/mL |

| Small Molecules | | |
|---|---|---|
| SF1670 (PTEN inhibitor) | 500 | nM |

Cultures were incubated at 3% oxygen (controlled by nitrogen) and 5% CO₂.

Small molecule components were added separately and fresh each time the media needs to be refreshed. Cytokines can be stored together. Media renewal should occur at least every few days.

On the days indicated one-half of the volume of the cell culture was removed for data analysis (flow cytometry using a BD FACS ARIA II). The culture volume was replenished with fresh media according to the conditions tested. The data reported accounts for the dilution factor introduced in this procedure.

Results

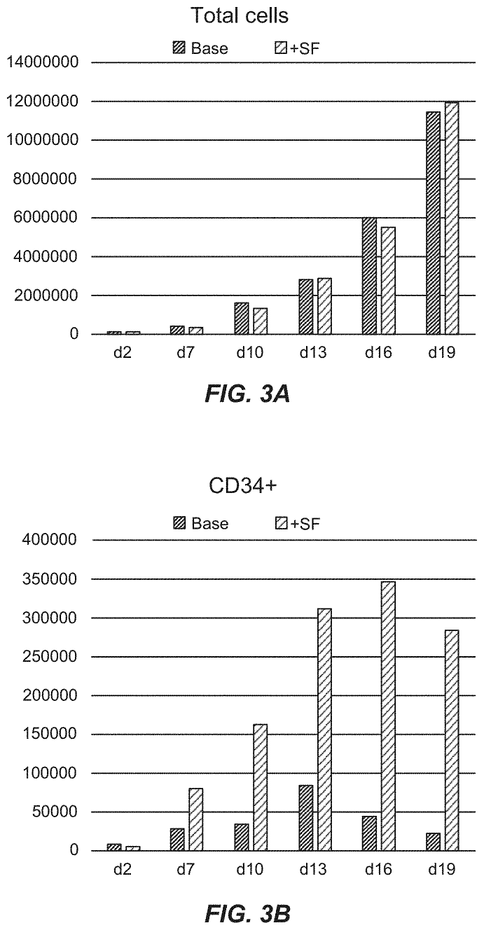

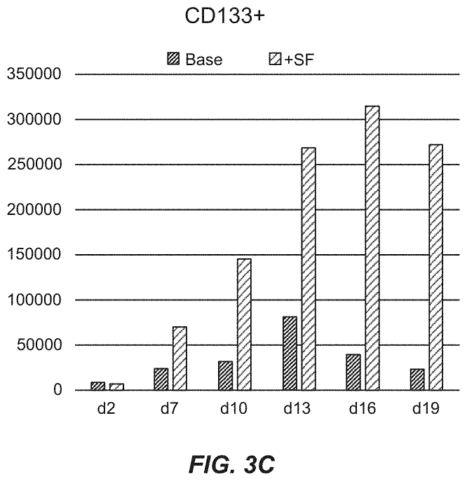

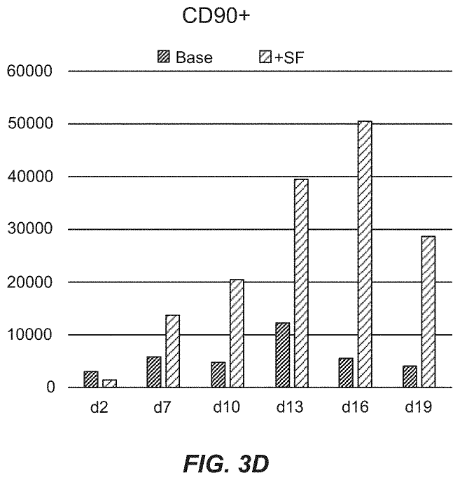

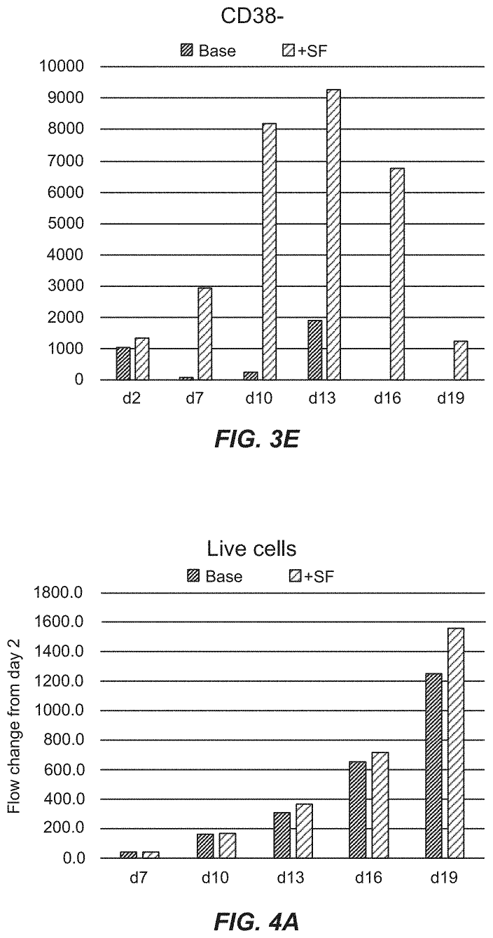

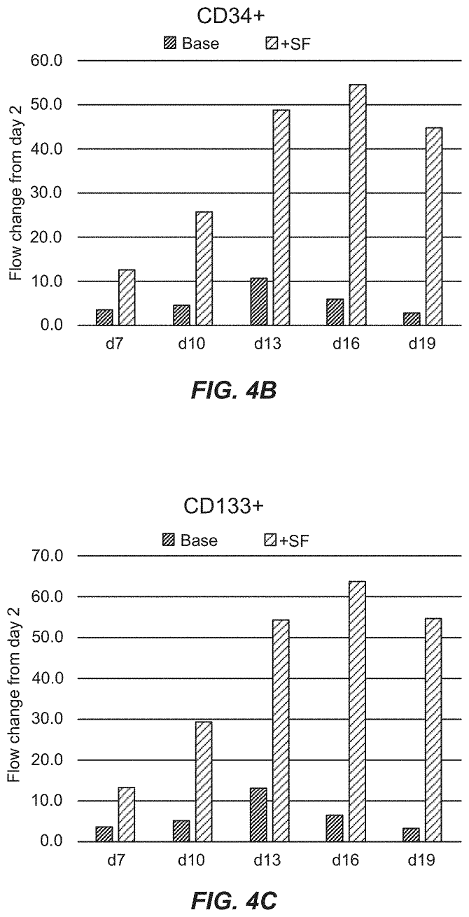

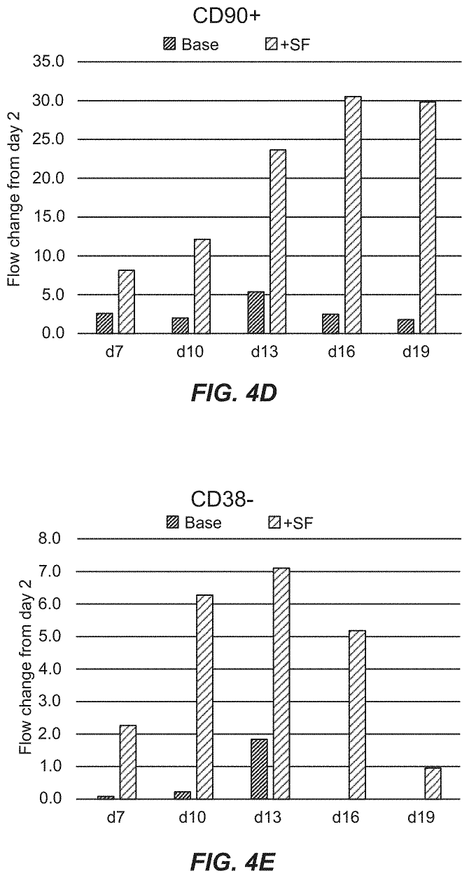

Flow cytometric analysis of +SF Conditions demonstrates that hematopoietic stem cells are maintained and continue to expand even after multiple days in culture (FIG. 3A-E). In fact, FIG. 4A-4E shows that after 16 days in culture there is a greater than 50-fold increase in CD34+ cells (FIG. 4B) and CD133+ cells (FIG. 4C) from day 2, about a 30-fold increase in CD90+ cells (FIG. 4D) from day 2, and about a 5-fold increase in CD38− cells (FIG. 4E) from day 2 in cord blood samples cultured in the presence of SF1670. These levels are markedly improved over cord blood samples cultured without SF1670 (base conditions). Compare, dark grey base conditions columns (on the left) with light grey +SF conditions (on the right) of FIGS. 3A-3E and FIGS. 4A-4E.

### Example 20: Titration of SF1670 and its Effect on the Enhancement of Hematopoietic Stem Cells Derived from Non-Mobilized Peripheral Blood in Culture

This Example illustrates the efficacious range of the positive expansive effect provided by SF1670.

### Materials and Methods

CD34+ cells were isolated from donor peripheral blood. Standard ficoll paque layering was used to separate the buffy coat. Cells then undergo negative depletion using biotinylated CD2, CD3, CD4, CD5, CD8, CD1 Ib, CD14, CD16, CD19, CD20, CD45RA, CD56, CD235 (in some examples CD15, CD25 and other lineage specific antibodies may also be used). Cells which bind these antibodies are depleted using streptavidin beads. The depleted cell pool was sorted for CD34+ cells before being placed in cell culture.

Isolated CD34+ cells were incubated in an in vitro culture media of Alpha MEM without phenol red, 10% (v/v) heat inactivated fetal bovine serum (FBS) and +SF conditions. +SF conditions included the components described in Table 5. The culture also included an antibiotic solution that includes penicillin, streptomycin, and amphotericin B to avoid contamination.

TABLE 5
Additional Components included in the culture
media of Base Conditions, +SF Conditions.

| | - Factor - | - Concentration - |
|---|---|---|
| +SF | Cytokines/Growth Factors | |
| Conditions | TPO | 100 ng/mL |
| | SCF | 100 ng/mL |
| | FLT3L | 100 ng/mL |
| | Small Molecules | |
| | SF1670 (PTEN inhibitor) | 0, 125 nM, 250 nM, 500 nM, |
| | | 1000 nM, or 2000 nM |

Cultures were incubated at 3% oxygen (controlled by nitrogen) and 5% CO₂.

Small molecule components were added separately and fresh each time the media needs to be refreshed. Cytokines can be stored together. Media renewal should occur at least every few days.

On the days indicated one-half of the volume of the cell culture was removed for data analysis (flow cytometry using a BD FACS ARIA II). The culture volume was replenished with fresh media according to the conditions tested. The data reported accounts for the dilution factor introduced in this procedure.

Results

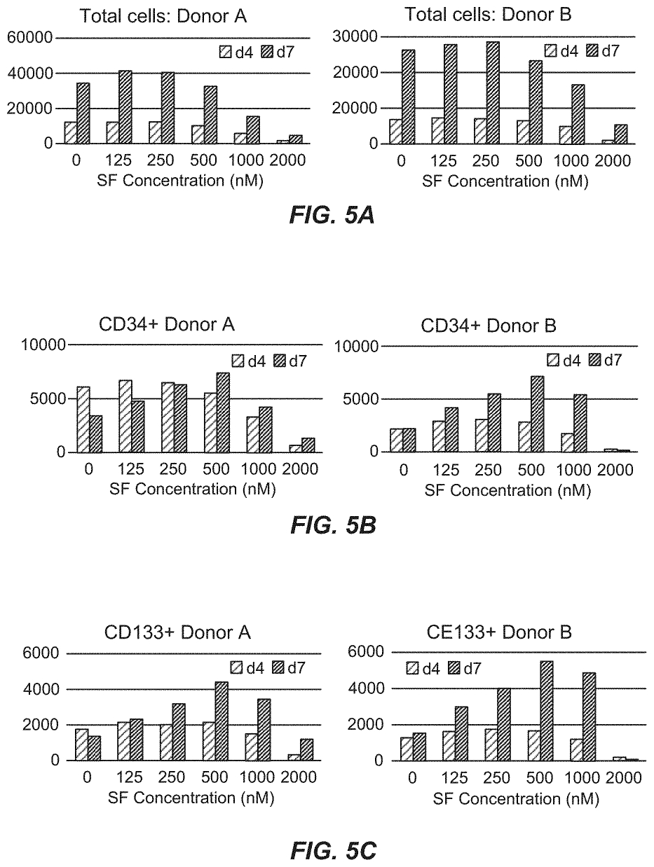

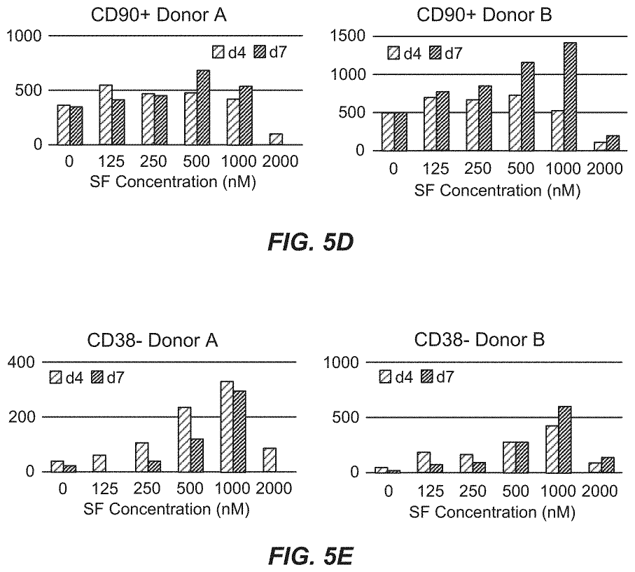

Flow cytometric analysis of demonstrates that at concentrations of 1000 nM, SF1670 provides a reduced expansive effect (FIG. 5A-5E). In fact, the 1000 nM concentration produced very little expansion of any cell type (FIG. 5A). All lower concentrations tested (125 nM, 250 nM, and 500 nM) provided at least some expansive effect on the enhancement of hematopoietic stem cells over the control condition (0 nM SF1670) (FIG. 5B-5E).

### Example 21: Enhancement of Hematopoietic Stem Cells Derived from Non-Mobilized Peripheral Blood Using Substituted Derivatives of 9,10-Dihydrophenathrene and Phenathrene

This Example demonstrates the enhancement of HSCs in cultures with chemically altered versions of SF1670 (substituted derivatives of 9,10-dihydrophenathrene and phenathrene).

### Materials and Methods

CD34+ cells were isolated from donor peripheral blood as described in Example 20. Isolated CD34+ cells were incubated in an in vitro culture media of Alpha MEM without phenol red, 10% (v/v) heat inactivated fetal bovine serum (FBS). When testing the substituted derivatives of 9,10-dihydrophenathrene and phenanthrene (+Derivative conditions), two internal controls were used: a positive control (+SF conditions) and a baseline control (i.e, basic conditions (“cytokines only”)). The media components and concentrations used for the compounds tested are described in Table 6. The culture also included an antibiotic solution that includes penicillin, streptomycin, and amphotericin B to avoid contamination. Controls were included because the amount of expansion in samples obtained varies from individual to individual.

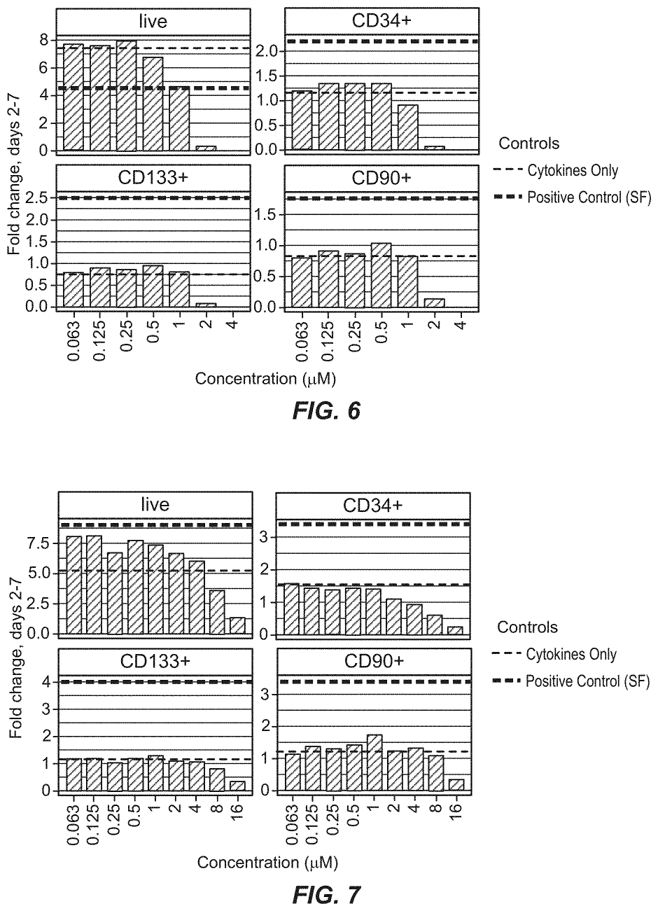

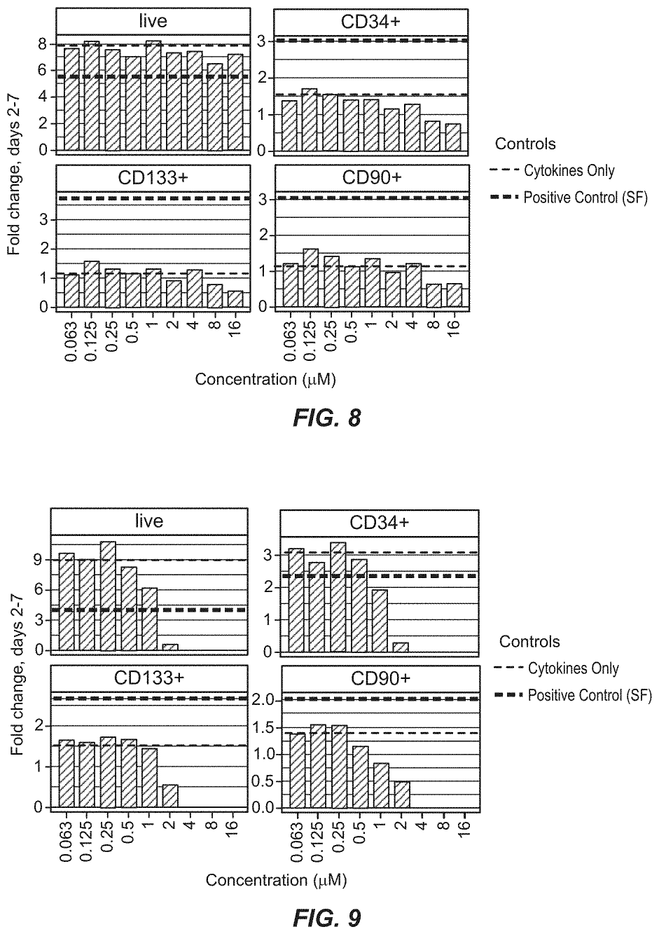

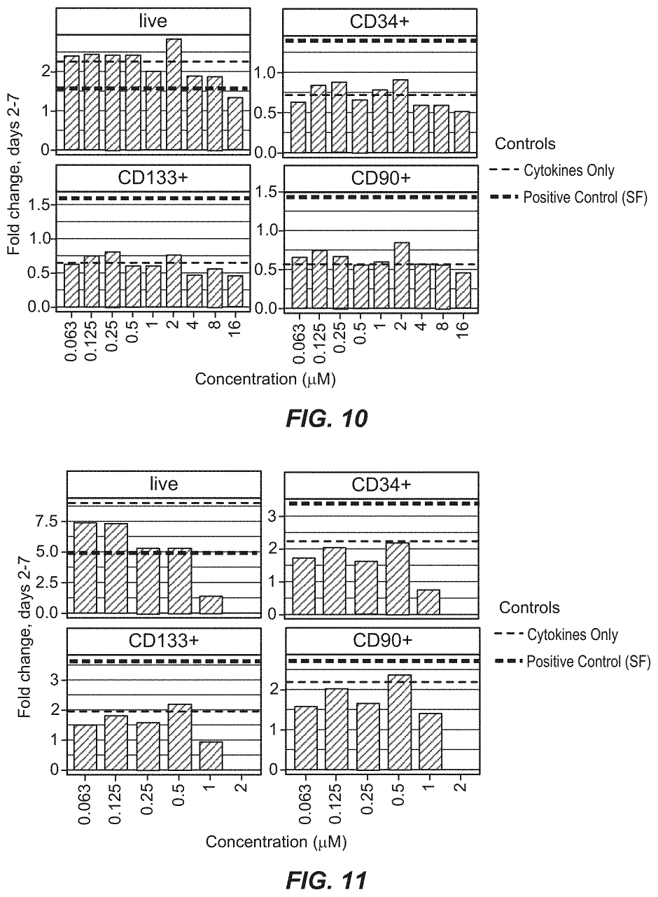

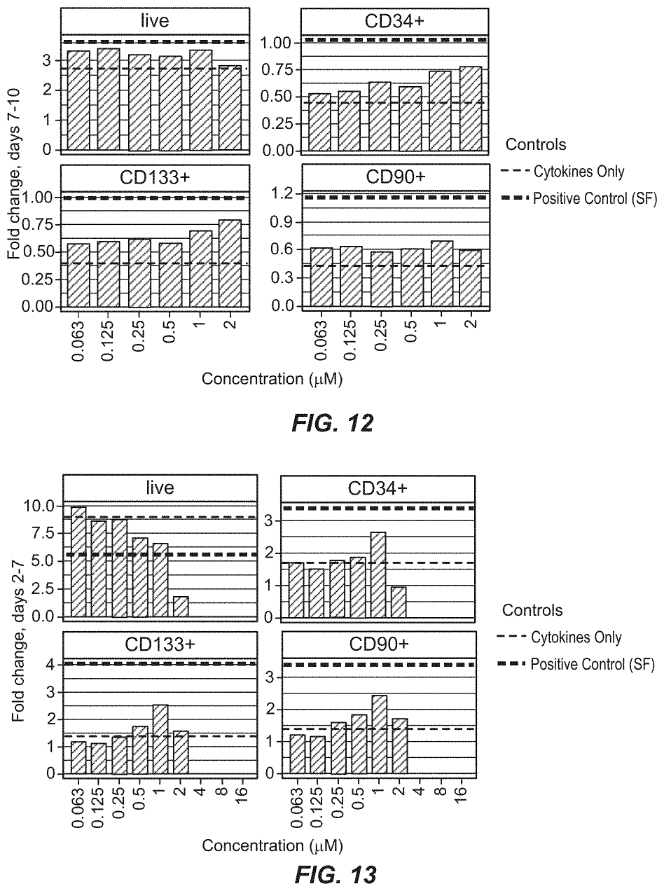

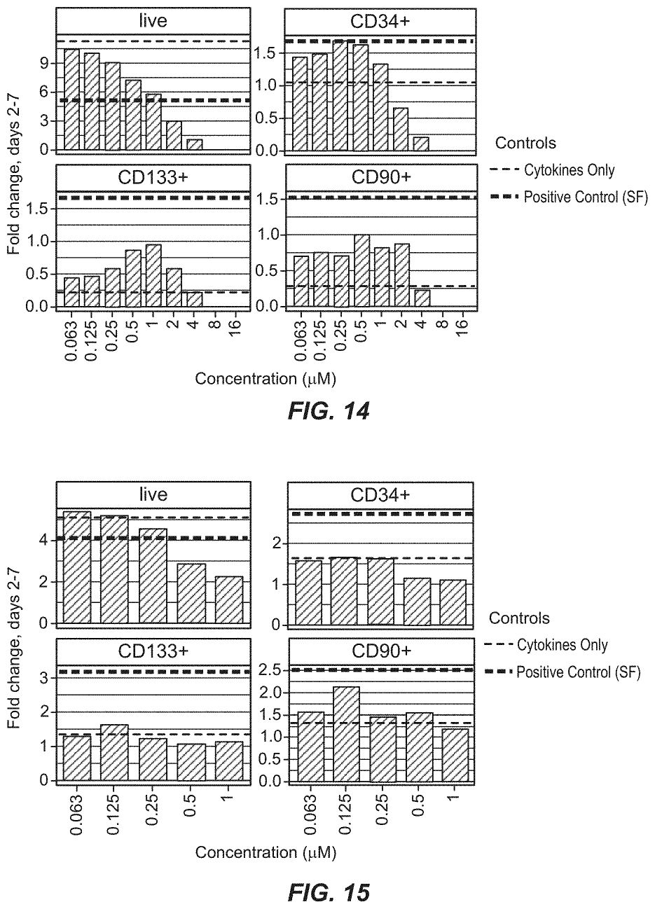

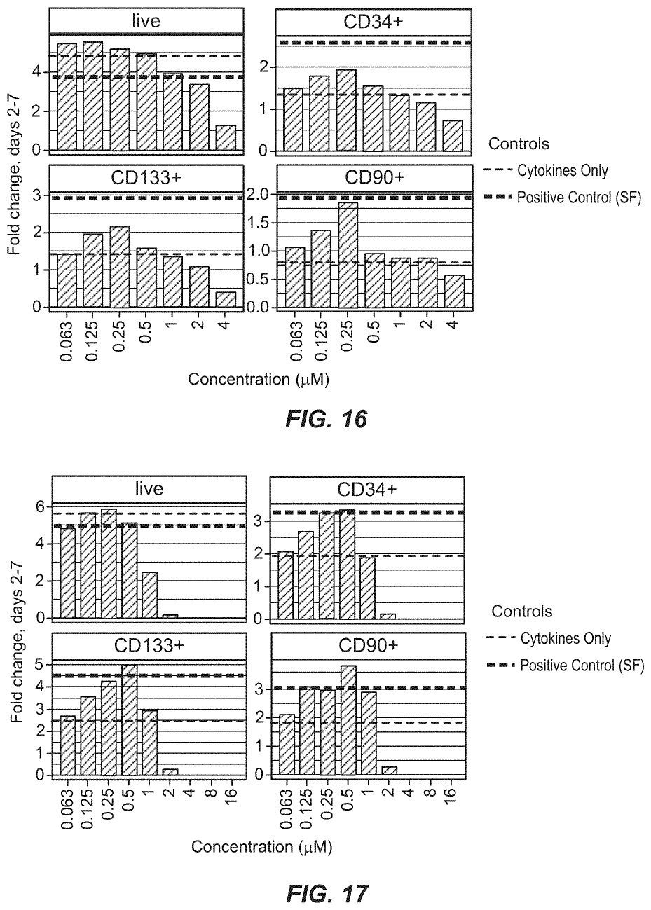

TABLE 6
Additional Components included in the culture media
of Basic Conditions (cytokines only); positive control
(+SF conditions); and Derivative conditions (substituted
derivatives of 9,10-dihydrophenathrene tested).

| | - Factor - | - Concentration - | |
|---|---|---|---|
| Base Conditions | Cytokines/Growth Factors | | |
| (Cytokines Only) | TPO | 100 | ng/mL |
| | SCF | 100 | ng/mL |
| | FLT3L | 100 | ng/mL |
| +SF Conditions | Cytokines/Growth Factors | | |
| (Positive Control) | TPO | 100 | ng/mL |
| | SCF | 100 | ng/mL |
| | FLT3L | 100 | ng/mL |
| | Small Molecules | | |
| | SF1670 (PTEN inhibitor) | 500 | nM |
| +Derivative | Cytokines/Growth Factors | | |
| Conditions | TPO | 100 | ng/mL |
| | SCF | 100 | ng/mL |
| | FLT3L | 100 | ng/mL |

| | |
|---|---|
| Small Molecules | |
| Substituted derivative of | 63 nM, 125 nM, 250 nM, |
| 9,10-dihydrophenathrene | 500 nM, 1 μM or as |
| | indicated in FIGs. 6-17. |

Cultures were incubated at 3% oxygen (controlled by nitrogen) and 5% CO₂.

Small molecule components were added separately and fresh each time the media needs to be refreshed. Cytokines can be stored together. Media renewal should occur at least every few days.

On the days indicated one-half of the volume of the cell culture was removed for data analysis (flow cytometry using a BD FACS ARIA II). The culture volume was replenished with fresh media according to the conditions tested. The data reported accounts for the dilution factor introduced in this procedure.

Separate experiments were performed for each compound tested (Compound 1.001-1.012).

Results

The expansive effect of Compounds 1.001 to 1.012 are displayed in FIG. 6-FIG. 17. The graphs in each figure report the fold change in cells between days 2 and 7. Each column in the figures report the fold change in cells at the noted concentration of derivative compound tested. The thin dashed line reports the expansive effect of the basic conditions (i.e. cytokines only), and thick dashed line reports the expansive effect of the +SF conditions (500 nM SF1670). Collectively, these data demonstrate that substituted derivatives of 9,10-dihydrophenathrene provide a positive expansive effect on HSCs in culture.

Table 7, below, summarizes the relative expansive effect of Compound 1.001 to 1.012 (sample compounds) at the indicated concentration. The data in Table 7 is reported as the relative expansive effect. The relative expansive effect is a normalized value of the fold changes shown in each of the figures. It is calculated as shown below:

$$\frac{\begin{matrix}Sample Compound Fold change - \\ Basic Conditions Fold Change\end{matrix}}{+ SF Conditions Fold Change - Basic Conditions Fold Change} = Relative Fold Change$$

TABLE 7
Relative expansive effect of CD133+ cells and
CD90+ cells in cultures containing Compounds
1.001-1.012 (sample compounds) at the indicated concentrations.

| | Concentration of | | |
|---|---|---|---|
| | sample compound | CD133 | CD90 |
| Compound | (μM) | effect | effect |
| 1.001 | 0.5 | + | ++ |
| 1.002 | 1 | + | ++ |
| 1.003 | 0.125 | + | ++ |
| 1.004 | 0.125 | + | ++ |
| 1.005 | 2 | + | ++ |
| 1.006 | 0.5 | + | ++ |
| 1.007 | 1 | ++ | ++ |
| 1.008 | 1 | ++ | ++ |
| 1.009 | 0.5 | ++ | +++ |
| 1.010 | 0.125 | + | +++ |
| 1.011 | 0.25 | ++ | ++++ |
| 1.012 | 0.5 | ++++ | +++++ |

The reported values (e.g. +, ++, and +++) for relative expansive effect of CD133 and CD90 cells presented in Table 7 are shown below, where “x” is the calculated relative fold-change.

| Relative Fold Change | Value |
|---|---|
| x < 0.2 | + |
| 0.2 ≤ x < 0.55 | ++ |
| 0.55 ≤ x < 0.9 | +++ |
| 0.9 ≤ x < 1.25 | ++++ |
| 1.25 ≤ x | +++++ |

### Example 22: Enhancement of Hematopoietic Stem Cells Derived from Non-Mobilized Peripheral Blood Using Substituted Derivatives of 9,10-Dihydrophenathrene and Phenathrene

This Example further demonstrates the enhancement of HSCs in cultures with chemically altered versions of SF1670 (substituted derivatives of 9,10-dihydrophenathrene and phenathrene).

### Materials and Methods

CD34+ cells were isolated from donor peripheral blood as described in Example 20. Isolated CD34+ cells were incubated in an in vitro culture media of Alpha MEM without phenol red, 10% (v/v) heat inactivated fetal bovine serum (FBS). When testing the substituted derivatives of 9,10-dihydrophenathrene (+Derivative conditions), two internal controls were used: a positive control (+SF conditions) and a baseline control (i.e, basic conditions (“cytokines only”)). The media components and concentrations used for the compounds tested are described in Table 8. The culture also included an antibiotic solution that includes penicillin, streptomycin, and amphotericin B to avoid contamination. Controls were included because the amount of expansion in samples obtained varies from individual to individual.

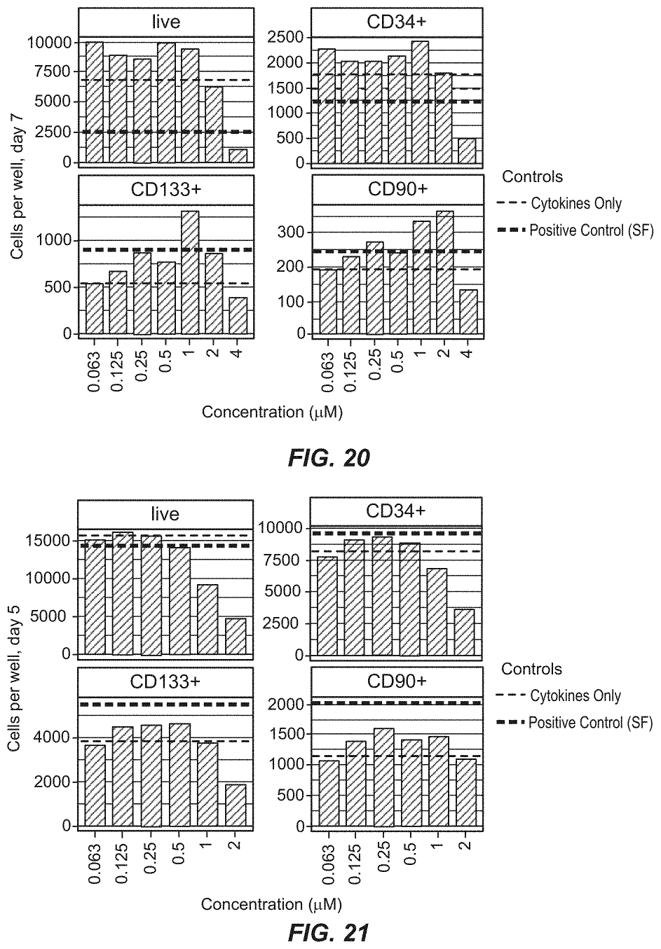

TABLE 8
Additional Components included in the culture media
of Basic Conditions (cytokines only); positive control
(+SF conditions); and Derivative conditions (substituted
derivatives of 9,10-dihydrophenathrene tested).

| | - Factor - | - Concentration - | |
|---|---|---|---|
| Base Conditions | Cytokines/Growth Factors | | |
| (Cytokines Only) | TPO | 100 | ng/mL |
| | SCF | 100 | ng/mL |
| | FLT3L | 100 | ng/mL |
| | IL-6 | 100 | ng/mL |
| +SF Conditions | Cytokines/Growth Factors | | |
| (Positive Control) | TPO | 100 | ng/mL |
| | SCF | 100 | ng/mL |
| | FLT3L | 100 | ng/mL |
| | IL-6 | 100 | ng/mL |
| | Small Molecules | | |
| | SF1670 (PTEN inhibitor) | 500 | nM |
| +Derivative | Cytokines/Growth Factors | | |
| Conditions | TPO | 100 | ng/mL |
| | SCF | 100 | ng/mL |
| | FLT3L | 100 | ng/mL |
| | IL-6 | 100 | ng/mL |

| | |
|---|---|
| Small Molecules | |
| Substituted derivative of | 63 nM, 125 nM, 250 |
| 9,10-dihydrophenathrene | nM, 500 nM, 1 μM, |
| | 2 μM, 4 μM or as |
| | indicated in FIGs. 17-21. |

Cultures were incubated at 3% oxygen (controlled by nitrogen) and 5% CO₂.

After the indicated number of days one-half of the volume of the cell culture was removed for data analysis (flow cytometry using a BD FACS ARIA II). The culture volume was replenished with fresh media according to the conditions tested.

Separate experiments were performed for each compound tested (Compound 1.014-1.016).

Results

The expansive effect of Compounds 1.014 to 1.016 are displayed in FIG. 19-FIG. 21. The graphs in each figure report the number of cells per well after the indicated number of days in culture. The thin dashed line reports the expansive effect of the basic conditions (i.e. cytokines only), and thick dashed line reports the expansive effect of the +SF conditions (500 nM SF1670). This data demonstrates that these substituted derivatives of 9,10-dihydrophenathrene provide a positive expansive effect on HSCs in culture.

Table 9, below, summarizes the relative expansive effect of Compound 1.014 to 1.016 (sample compounds) at the indicated concentration. The data in Table 9 is reported as the relative cell number. The relative cell number is the normalized value of cell counts after the indicated number of days in culture. It is calculated as shown below:

$$\frac{\begin{matrix}Sample Compound Cell Count - \\ Basic Conditions Cell Count\end{matrix}}{+ SF Conditions Cell Count - Basic Conditions Cell Count} = Relative Cell number$$

TABLE 9
Relative expansive effect of CD133+ cells and
CD90+ cells in cultures containing Compounds
1.014-1.016 (sample compounds) at the indicated concentrations.

| | | Concentration of | | |
|---|---|---|---|---|
| | Days in | sample compound | CD133 | CD90 |
| Compound | culture | (μM) | effect | effect |
| 1.014 | 7 | 1 | ++ | ++ |
| 1.015 | 7 | 2 | +++ | +++++ |
| 1.016 | 5 | 0.25 | ++ | ++ |

The reported values (e.g. +, ++, and +++) for relative expansive effect of CD133 and CD90 cells presented in Table 9 are shown below, where “x” is the calculated relative expansive effect.

| Relative Fold Change | Value |
|---|---|
| x < 0.2 | + |
| 0.2 ≤ x < 0.55 | ++ |
| 0.55 ≤ x < 0.9 | +++ |
| 0.9 ≤ x < 1.25 | ++++ |
| 1.25 ≤ x | +++++ |

### Example 23: Enhancement of Hematopoietic Stem Cells Derived from Mobilized Peripheral Blood Using Substituted Derivatives of 9,10-dihydrophenathrene in Alternative Base Media

This Example demonstrates the enhancement of HSCs in cultures derived from mobilized peripheral blood with chemically altered versions of SF1670 (substituted derivatives of 9,10-dihydrophenathrene and phenathrene) in a different base media.

### Materials and Methods

Mobilized peripheral blood was purchased from StemCell Technologies. The blood was mobilized using G-CSF from volunteer donors. Volunteers were administered a maximum of 10 ug/kg/day of granulocyte colony-stimulating factor (G-CSF) for 3-5 days prior to collection. Primary human CD34+ cells were isolated from mobilized peripheral blood leukapheresis samples using positive immunomagnetic separation techniques.

Isolated CD34+ cells were incubated in an in vitro culture media of StemSpan SFEM. When testing the substituted derivatives of 9,10-dihydrophenathrene (+Derivative conditions), two internal controls were used: a positive control (+SF conditions) and a baseline control (i.e, basic conditions (“cytokines only”)). The media components and concentrations used for the compounds tested are described in Table 8, above. The culture also included an antibiotic solution that includes penicillin, streptomycin, and amphotericin B to avoid contamination. Controls were included because the amount of expansion in samples obtained varies from individual to individual.

Cultures were incubated at 3% oxygen (controlled by nitrogen) and 5% CO₂.

After the indicated number of days one-half of the volume of the cell culture was removed for data analysis (flow cytometry using a BD FACS ARIA II). The culture volume was replenished with fresh media according to the conditions tested.

Results

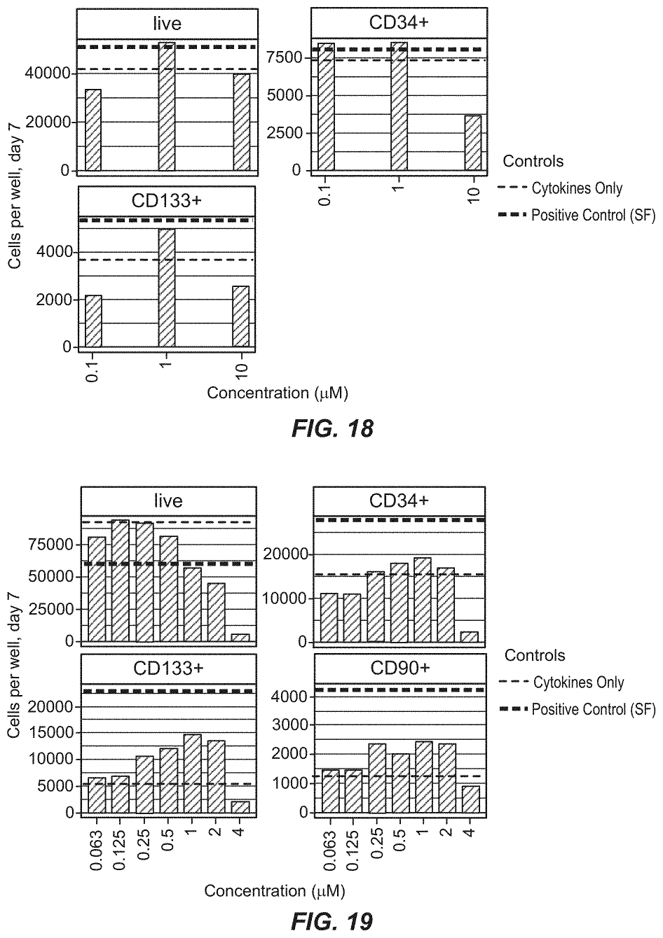

The expansive effect of Compound 1.013 is displayed in FIG. 18. The graphs report the number of cells per well after the indicated number of days in culture. The thin dashed line reports the expansive effect of the basic conditions (i.e. cytokines only), and thick dashed line reports the expansive effect of the +SF conditions (500 nM SF1670). This data demonstrates that Compound 1.013 provides a positive expansive effect on HSCs in culture, and further shows that different sources of CD34+ cells and base media can be used.

Table 10, below, summarizes the relative expansive effect of Compound 1.013 at the indicated concentration. The data in Table 10 is reported as the relative cell number. Relative cell number is calculated as described in Example 22.

TABLE 10
Relative expansive effect of CD133+ cells in cultures containing
Compound 1.013 (sample compounds) at the indicated concentrations.

| | | Concentration of | |
|---|---|---|---|
| | Days in | sample compound | CD133 |
| Compound | culture | (μM) | effect |
| 1.013 | 7 | 1 | +++ |

The reported values (e.g. +, ++, and +++) for relative expansive effect of CD133 cells presented in Table 10 are as defined in Example 22.

Although the foregoing invention has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference. Where a conflict exists between the instant application and a reference provided herein, the instant application shall dominate.

1. A method for expanding hematopoietic stem cells in culture, the method comprising contacting a source of CD34+ cells in culture with an effective amount of a phosphatase and tensin homolog (PTEN) inhibitor, thereby expanding hematopoietic stem cells in the culture.

2.-6. (canceled)

7. The method of claim 1, wherein the PTEN inhibitor is a compound of Formula I 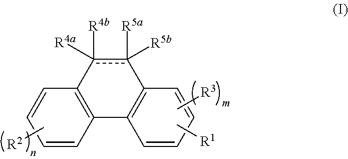
or a pharmaceutically acceptable salt, hydrate, or solvate thereof; wherein
the dashed line (represented by - - - - ) is an optional double bond;
R¹ is selected from the group consisting of —C(O)—NR^{b}—R^{1a}, —NR^{b}—C(O)—R^{1a}, —NR^{b}—C(O)—R^{1b}, —NR^{b}—X¹—C(O)—R^{1a}, —C(O)—X¹—NR^{b}—R^{1a}, —X¹—C(O)—NR^{b}—R^{1a}, —X¹—NR^{b}—C(O)—R^{1a}, —NR^{b}—C(O)—X¹—C(O)—R^{1b}, —C(O)—NR^{b}—X¹—C(O)—R^{1b}, —NR^{b}—C(O)—O—R^{1a}, —NR^{b}—C(O)—O—R^{1b}, —O—C(O)—NR^{b}—R^{1a}, —X¹—NR^{b}—C(O)—O—R^{1a}, —X¹—O—C(O)—NR^{b}—R^{1a}, —O—R^{1a}, —NR^{b}—R^{1a}, —NR^{b}—C(O)—X¹—O—X¹—R^{1a} and —C(O)—R^{1a};
R^{1a} is selected from the group consisting of H, C₁₋₁₀ alkyl, C₁₋₁₀ haloalkyl, and phenyl;
R^{1b} is selected from the group consisting of —OR^{c}, —NR^{a}R^{b};
each R² is independently selected from the group consisting of halogen, —CN, —C₁₋₈ alkyl, —C₂₋₈ alkenyl, C₂₋₈ alkynyl, C₁₋₈ haloalkyl, —C₁₋₈ alkoxy, —X¹—C₁₋₈ alkoxy, —C(O)—R^{2a}, —SR^{a}, —X¹—SR^{a}, —OR^{a}, —X¹—OR^{a}, —NR^{a}R^{b}, —X¹—NR^{a}R^{b}, —S(O)₂R^{a}, —S(O)₂NR^{a}R^{b}, —X¹—S(O)₂R^{a}, and —X¹—S(O)₂NR^{a}R^{b};
each R^{2a} is independently selected from the group consisting of H, —C₁₋₁₀ alkyl, —C₁₋₁₀ haloalkyl, —OR^{a}, —X¹—OR^{a}, —NR^{a}R^{b}, and —X¹—NR^{a}R^{b};
each R³ is independently selected from the group consisting of halogen, —CN, —C₁₋₈ alkyl, —C₂₋₈ alkenyl, —C₂₋₈ alkynyl, —C₁₋₈ haloalkyl, —C₁₋₈ alkoxy, —C(O)—R^{3a}, —SR^{a}, —X¹—SR^{a}, —OR^{a}, —X¹—OR^{a}, —NR^{a}R^{b}, —X¹—NR^{a}R^{b}, —S(O)₂R^{a}, —S(O)₂NR^{a}R^{b}, —X¹—S(O)₂R^{a}, and —X¹—S(O)₂NR^{a}R^{b};
each R^{3a} is independently selected from the group consisting of H, C₁₋₁₀ alkyl, C₁₋₁₀ haloalkyl, —OR^{a}, —X¹—OR^{a}, —NR^{a}R^{b}, and —X¹—NR^{a}R^{b};
R^{4a} is selected from the group consisting of —OR^{c} and —NR^{c}R^{d};
R^{4b} is H; or R^{4a} and R^{4b} are combined to form an oxo or oxime moiety;
R^{5a} is selected from the group consisting of —OR^{c} and —NR^{c}R^{d};
R^{5b} is H; or R^{5a} and R^{5b} are combined to form an oxo or oxime moiety;
when either R^{4a} and R^{4b} or R^{5a} and R^{5b} combine to form an oxo or oxime moiety, the dashed line is absent;
each R^{a} and R^{b} is independently selected from the group consisting of H and C₁₋₄ alkvl;
each R^{c} and R^{d} is independently selected from the group consisting of H, —C₁₋₈ alkyl, —C₂₋₈ alkenyl, —C₂₋₈ alkynyl, —C₁₋₈ haloalkyl, —X¹—SR^{a}, —X¹—OR^{a}, —NR^{a}R^{b}, —X¹—NR^{a}R^{b}, —C(O)—H, —C(O)—C₁₋₈alkyl, C₃₋₆ cycloalkyl, heterocycloalkyl, aryl, and heteroaryl, wherein
the heterocycloalkyl group is a 4 to 6 membered ring having 1-3 heteroatom ring vertices selected from the group consisting of O, N, and S;
wherein the heteroaryl group is a 5 to 6 membered ring having 1-3 heteroatom ring vertices selected from the group consisting of O, N, and;
each X¹ is independently C₁₋₄ alkylene;
the subscript n is an integer from 0 to 3; and
the subscript m is an integer from 0 to 2.

8. The method of claim 1, further comprising a retinoic acid receptor (RAR) inhibitor or modulator.

9. The method of claim 8, wherein the retinoic acid receptor (RAR) inhibitor or modulator is ER50891.

10.-19. (canceled)

20. A medium for expanding hematopoietic stem cells in culture comprising:
(a) (i) a base medium or (ii) a feed medium; and
(b) a phosphatase and tensin homolog (PTEN) inhibitor.

21.-56. (canceled)

57. A population of hematopoietic stem cells produced by the method of claim 1.

58. A therapeutic agent comprising the population of hematopoietic stem cells of claim 57.

59. A method of treating an individual in need of hematopoietic reconstitution, comprising administering to said individual the therapeutic agent of claim 58.

60.-77. (canceled)

78. A compound of Formula I 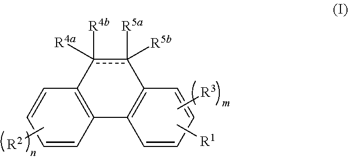
or a pharmaceutically acceptable salt, hydrate, or solvate thereof; wherein
the dashed line (represented by - - - - ) is an optional double bond;
R¹ is selected from the group consisting of —C(O)—NR^{b}—R^{1a}, —NR^{b}—C(O)—R^{1a}, —NR^{b}—C(O)—R^{1b}, —NR^{b}—X₁—C(O)—R^{1a}, —C(O)—X¹—NR^{b}—R^{1a}, —X¹—C(O)—NR^{b}—R^{1a}, —X¹—NR^{b}—C(O)—R^{1a}, —NR^{b}—C(O)—X¹—C(O)—R^{1b}, —C(O)—NR^{b}—X¹—C(O)—R^{1b}, —NR^{b}—C(O)—O—R^{1a}, —NR^{b}—C(O)—O—R^{1b}, —O—C(O)—NR^{b}—R^{1a}, —X¹—NR^{b}—C(O)—O—R^{1a}, —X¹—O—C(O)—NR^{b}—R^{1a}, —O—R^{1a}, —NR^{b}—R^{1a}, —NR^{b}—C(O)—X¹—O—X¹—R^{1a} and —C(O)—R^{1a};
R^{1a} is selected from the group consisting of H, C¹⁻¹⁰ alkyl, C¹⁻¹⁰ haloalkyl, and phenyl;
R^{1b} is selected from the group consisting of —OR^{c}, —NR^{a}R^{b};
each R² is independently selected from the group consisting of halogen, —CN, —C₁₋₈ alkyl, —C₂₋₈ alkenyl, C₂₋₈ alkynyl, C₁₋₈ haloalkyl, —C₁₋₈ alkoxy, —X¹—C₁₋₈ alkoxy, —C(O)—R^{2a}, —SR^{a}, —X¹—SR^{a}, —OR^{a}, —X¹—OR^{a}, —NR^{a}R^{b}, —X¹—NR^{a}R^{b}, —S(O)₂R^{a}, —S(O)₂NR^{a}R^{b}, —X¹—S(O)₂R^{a}, and —X¹—S(O)₂NR^{a}R^{b};
each R^{2a} is independently selected from the group consisting of H, —C₁₋₁₀ alkyl, —C₁₋₁₀ haloalkyl, —OR^{a}, —X¹—OR^{a}, —NR^{a}R^{b}, and —X¹—NR^{a}R^{b};
each R³ is independently selected from the group consisting of halogen, —CN, —C₁₋₈ alkyl, —C₂₋₈ alkenyl, —C₂₋₈ alkynyl, —C₁₋₈ haloalkyl, —C₁₋₈ alkoxy, —C(O)—R^{3a}, —SR^{a}, —X¹—SR^{a}, —OR^{a}, —X¹—OR^{a}, —NR^{a}R^{b}, —X¹—NR^{a}R^{b}, —S(O)₂R^{a}, —S(O)₂NR^{a}R^{b}, —X¹—S(O)₂R^{a}, and —X¹—S(O)₂NR^{a}R^{b};
each R^{3a} is independently selected from the group consisting of H, C₁₋₁₀ alkyl, C₁₋₁₀ haloalkyl, —OR^{a}, —X¹—OR^{a}, —NR^{a}R^{b}, and —X¹—NR^{a}R^{b};
R^{4a} is selected from the group consisting of —OR^{c} and —NR^{c}R^{d};
R^{4b} is H; or R^{4a} and R^{4b} are combined to form an oxo or oxime moiety;
R^{5a} is selected from the group consisting of —OR^{c} and —NR^{c}R^{d};
R^{5b} is H; or R^{5a} and R^{5b} are combined to form an oxo or oxime moiety;
when either R^{4a} and R^{4b} or R^{5a} and R^{5b} combine to form an oxo or oxime moiety, the dashed line is absent;
each R^{a} and R^{b} is independently selected from the group consisting of H and C₁₋₄ alkyl;
each R^{c} and R^{d} is independently selected from the group consisting of H, —C₁₋₈ alkyl, —C₂₋₈ alkenyl, —C₂₋₈ alkynyl, —C₁₋₈ haloalkyl, —X—SR^{a}, —X—OR^{a}, —NR^{a}R^{b}, —X¹—NR^{a}R^{b}, —C(O)—H, —C(O)—C₁₋₈alkyl, C₃₋₆ cycloalkyl, heterocycloalkyl, aryl, and heteroaryl, wherein
the heterocycloalkyl group is a 4 to 6 membered ring having 1-3 heteroatom ring vertices selected from the group consisting of O, N, and S;
wherein the heteroaryl group is a 5 to 6 membered ring having 1-3 heteroatom ring vertices selected from the group consisting of O, N, and;
each X¹ is independently C₁₋₄ alkylene;
the subscript n is an integer from 0 to 3; and
the subscript m is an integer from 0 to 2;
provided that the compound of Formula I is not N-(9,10-Dihydro-9,10-dioxo-2-phenanthrenyl)-2,2-dimethyl-propanamide.

79.-81. (canceled)

82. The compound of claim 78, wherein
R¹ is selected from the group consisting of —C(O)—NH—R^{1a}, —NH—C(O)—R^{1a}, —NH—X¹—C(O)—R^{1a}, and —NH—C(O)—X¹—C(O)—R^{b}.

83. The compound of claim 78, wherein
R¹ is —NH—C(O)—R^{1a}.

84.-88. (canceled)

89. The compound of claim 78, wherein
each R² and R³ is independently selected from the group consisting of —NR^{a}R^{b} and —X¹—NR^{a}R^{b}.

90. (canceled)

91. The compound of claim 78, wherein
R^{1a} is C₁₋₁₀ alkyl, C₁₋₁₀ haloalkyl, or phenyl.

92.-100. (canceled)

101. The compound of claim 78, wherein
the subscript n is 0.

102. (canceled)

103. The compound of claim 78, wherein
the subscript m is 0.

104. (canceled)

105. (canceled)

106. The compound of claim 78, wherein the compound of Formula I has the structure of Formula I-5 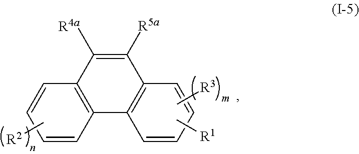
or a pharmaceutically acceptable salt, hydrate, or solvate thereof, wherein
R^{4a} is selected from the group consisting of —OR^{c}, and —NR^{c}R^{d};
R^{5a} is selected from the group consisting of —OR^{c}, and —NR^{c}R^{d}.

107.-110. (canceled)

111. The compound of claim 106, wherein R^{4a} and R^{5a}, are independently selected from the group consisting of —OH, —NH₂, —O—C(O)—CH₃, and —NH—C(O)—CH₃.

112.-114. (canceled)

115. The compound of claim 78, wherein the compound of Formula I has the structure of Formula II 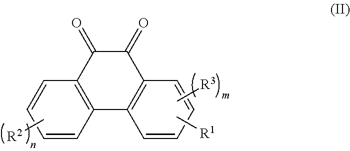
or a pharmaceutically acceptable salt, hydrate, or solvate thereof.

116.-119. (canceled)

120. The compound of claim 78, wherein the compound of Formula I has the structure of Formula III 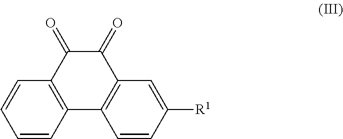
or a pharmaceutically acceptable salt, hydrate, or solvate thereof.

121. The compound of claim 78, wherein the compound of Formula I has the structure of Formula IIIa 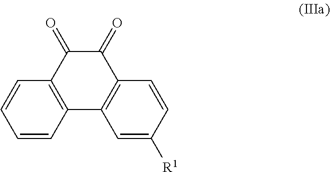
or a pharmaceutically acceptable salt, hydrate, or solvate thereof.

122. The compound of claim 120, wherein
R¹ is selected from the group consisting of —C(O)—NH—R^{1a}, —NH—C(O)—R^{1a}, —NH—X¹—C(O)—R^{1a}, and —NH—C(O)—X¹—C(O)—R^{1b};
R^{1a} is selected from the group consisting of C₁₋₁₀ alkyl, C₁₋₁₀ haloalkyl, and phenyl;
R^{1b} is OH;
R^{a} and R^{b} are independently selected from the group consisting of H and C₁₋₄ alkyl;
X¹ is C₁₋₂ alkylene; and
provided that the compound of Formula II is not N-(9,10-Dihydro-9,10-dioxo-2-phenanthrenyl)-2,2-dimethyl-propanamide.

123. The compound of claim 78, wherein said compound is selected from the group consisting of 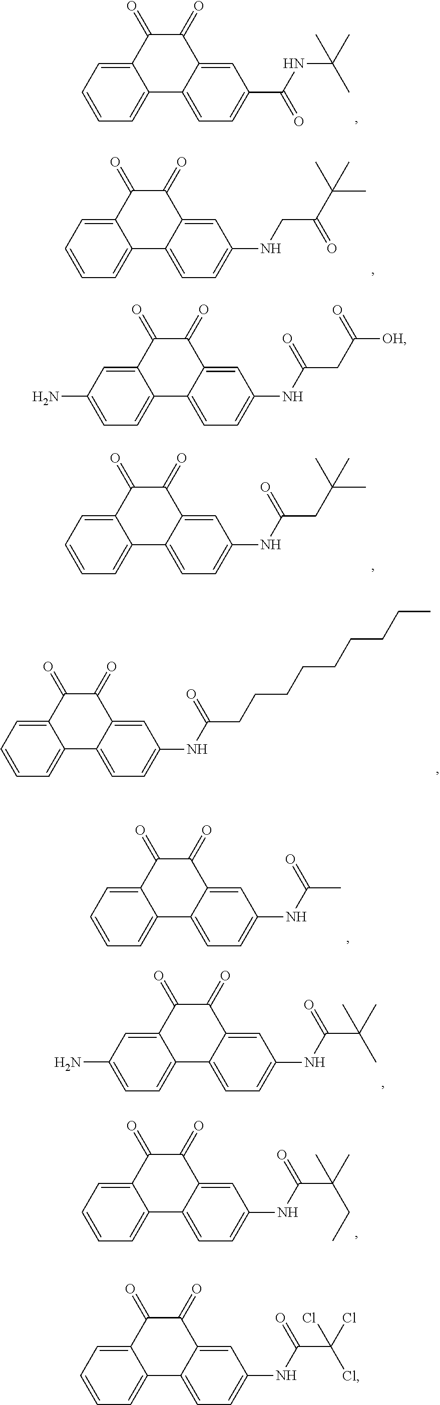 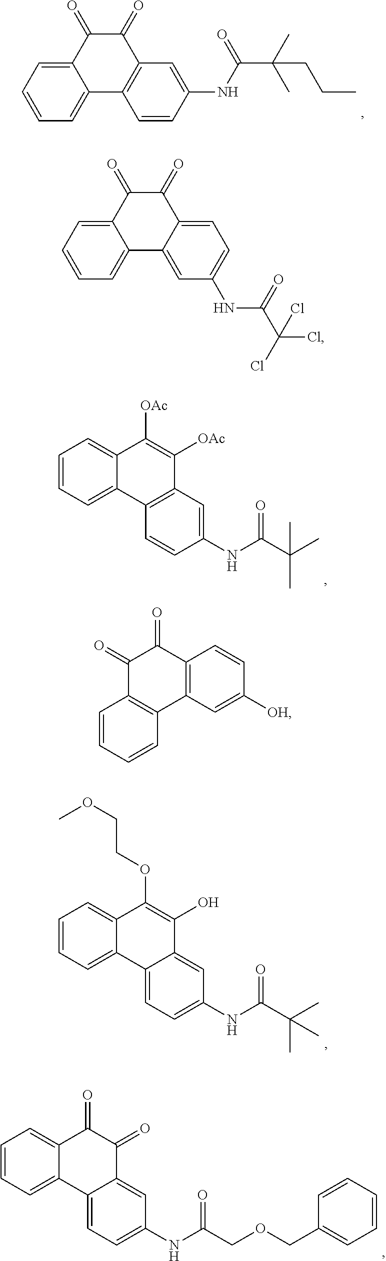 [chemical structure]
